(19) United States
(12) Patent Application Publication
(10) Pub. No.: US 2025/0205182 A1
(43) Pub. Date: Jun. 26, 2025

(54) COMPOSITIONS AND METHODS FOR IMPROVING GUT PERMEABILITY

(71) Applicant: BRIGHTSEED, INC., South San Francisco, CA (US)

(72) Inventors: Lee Heil Chae, San Francisco, CA (US); Danielle Aina-Badejo, Belmont, CA (US)

(21) Appl. No.: 19/075,230

(22) Filed: Mar. 10, 2025

Related U.S. Application Data
(63) Continuation of application No. PCT/US2023/033082, filed on Sep. 18, 2023.
(60) Provisional application No. 63/407,950, filed on Sep. 19, 2022.

(51) Int. Cl. A61K 31/165; A61K 9/00; A61P 1/00

(52) U.S. Cl. CPC A61K 31/165; A61K 9/0056; A61P 1/00

(57) ABSTRACT

Disclosed herein are compositions and methods for treating, improving or ameliorating a disease or condition associated with intestinal permeability, improving intestinal permeability, and improving gut barrier function in a subject. Some embodiments include, for example, providing or administering one or more compounds of Formula (I) or one or more compounds of Formula (II). Some embodiments provide the composition to be formulated as a dietary supplement, food ingredient or additive, food product, a medical food, nutraceutical or pharmaceutical composition.

### CROSS-REFERENCE TO RELATED APPLICATIONS

This application is a continuation of U.S. National Phase of International No. PCT/US2023/033082, filed Sep. 18, 2023, claims the benefit of priority to U.S. Provisional Application No. 63/407,950, filed Sep. 19, 2022, which is hereby incorporated herein by reference in its entirety for all purposes.

### FIELD

The present disclosure relates generally to the field of food and beverage supplements, food ingredient or additive, a medical food, nutraceutical or pharmaceutical composition. More specifically, the disclosure relates to methods and compositions to improve, maintain, and/or promote good metabolism, an immunomodulatory response, digestion, and gut health.

### BACKGROUND

Obesity is associated with increased gut permeability, a condition in which Hepatocyte nuclear factor 4 alpha (HNF4α), a nuclear transcription factor found in the intestinal lumen, has been shown to play a critical role. It impacts mucin production, a key component of the gut barrier, and regulates tight junction protein expression and Paneth cell differentiation.

### SUMMARY OF THE DISCLOSURE

Some aspects of the disclosure relate to compositions and methods for treating, improving or ameliorating a disease or condition associated with intestinal permeability, improving intestinal permeability, or improving impaired gut barrier function in a subject. In some embodiments, the method includes providing or administering to the subject in need thereof a composition comprising at least one carrier and an effective amount of a compound of Formula (I),

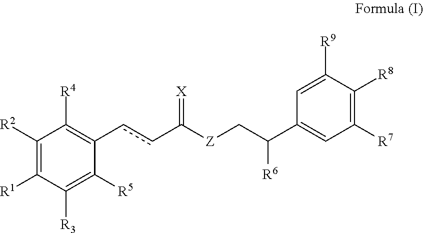

wherein
R¹, R², R³, R⁴, R⁵, R⁶, R⁷, R⁸, and R⁹ are each independently selected from hydrogen, deuterium, hydroxyl, halogen, cyano, nitro, optionally substituted amino, optionally substituted C-amido, optionally substituted N-amido, optionally substituted ester, optionally substituted —C₁₋₆alkyl, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl; the dashed bond is present or absent;
X is CH₂ or O;
Z is CHR^{a}, NR^{a}, or O; and
R^{a} is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl, thereby treating, improving or ameliorating a disease or condition associated with intestinal permeability in the subject.

In some embodiments, the composition is formulated as a dietary supplement, food ingredient or additive, a medical food, nutraceutical or pharmaceutical composition. In some embodiments, the composition of Formula (I) is in a unit dosage form and is configured for administration between 0.1 and 100 mg/kg of the body weight of the subject of Formula (I) per administration. In some embodiments, the compound of Formula (I) is selected from the group consisting of: N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, N-trans-chlorogenoyltyramine, N-trans-rosmarinoyltyramine, and 5-hydroxyferuloyltyramine, or a pharmaceutically acceptable salt, solvates, and combinations of the foregoing. In some embodiments, the disease or condition associated with intestinal permeability in the subject is associated with a gastrointestinal epithelial cell barrier function disorder. In some embodiments, the gastrointestinal epithelial cell barrier function disorder is a disease associated with decreased intestinal epithelium integrity. In some embodiments, the gastrointestinal epithelial cell barrier function disorder is at least one selected from the group consisting of: inflammatory bowel disease, Crohn's disease, ulcerative colitis, pouchitis, irritable bowel syndrome, enteric infections, Clostridium difficile infections, metabolic diseases, obesity, type 2 diabetes, non-alcoholic steatohepatitis, non-alcoholic fatty liver disease, liver disorders, alcoholic steatohepatitis, celiac disease, necrotizing enterocolitis, gastro intestinal disorders, short bowel syndrome, GI mucositis, chemotherapy induced mucositis, radiation induced mucositis, oral mucositis, interstitial cystitis, neurological disorders, cognitive disorders, Alzheimer's, Parkinson's, multiple sclerosis, autism, chemotherapy associated steatohepatitis (CASH), and pediatric versions of the aforementioned diseases. In some embodiments, administering comprises rectal, parenteral, intravenous, topical, oral, dermal, transdermal, or subcutaneous administration. In some embodiments, the subject experiences a reduction in at least one symptom associated with the gastrointestinal epithelial cell barrier function disorder selected from the group consisting of: abdominal pain, blood in stool, pus in stool, fever, weight loss, frequent diarrhea, fatigue, reduced appetite, tenesmus, and rectal bleeding. In some embodiments, the method further includes administering at least one second therapeutic agent to the subject, said second therapeutic agent selected from the group consisting of: an anti-diarrheal, a 5-aminosalicylic acid compound, an antiinflammatory agent, an antibiotic, an antibody, an anti-cytokine agent, an antiinflammatory cytokine agent, a steroid, a corticosteroid, and an immunosuppressant. In some embodiments, treating or improving a disease or condition associated with intestinal permeability in a subject improves digestive health in a subject by at least 10%. In some embodiments, treating or improving a disease or condition associated with intestinal permeability in a subject reduces a disease or condition in a subject by at least 10%. In some embodiments, the compound of Formula I is an extract of a plant. In some embodiments, the extract of the plant is selected from the group comprising Allium, Amoracia, Chenopodium, Canabus, Spinacia, Fagopyrum, Annona, Jatropha, Piper, Eragrostis, Zea, Nelumbo, Ipomoea, Capsicump, Lycium, Solanum, and Tribulus. In some embodiments, the extract of the plant is hemp hull or black seed.

Some aspects relate to a consumable composition for treating a disease or condition associated with intestinal permeability or improving impaired gut barrier function comprising at least one carrier and an effective amount of a compound of Formula (I) or a pharmaceutically acceptable salt:

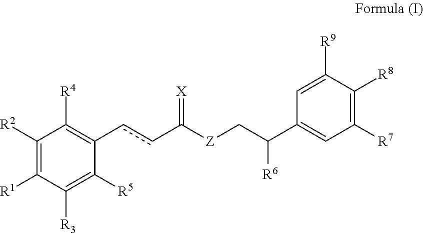

wherein,
R¹, R², R³, R⁴, R⁵, R⁶, R⁷, R⁸, and R⁹ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl; the dashed bond is present or absent;
X is CH₂ or O;
Z is CHR^{a}, NR^{a}, or O; and
R^{a} is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

In some embodiments, the composition is formulated as a dietary supplement, food ingredient or additive, a food product, a medical food, nutraceutical or pharmaceutical composition. In some embodiments, the composition of Formula (I) is in a unit dosage form and is configured for administration between 0.1 and 100 mg/kg of the body weight of the subject of Formula (I) per administration. In some embodiments, the compound of Formula (I) is selected from the group consisting of: N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, N-trans-chlorogenoyltyramine, N-trans-rosmarinoyltyramine, and 5-hydroxyferuloyltyramine, or a pharmaceutically acceptable salt, solvates, and combinations of the foregoing. In some embodiments, the compound of Formula (I) is N-trans-caffeoyltyramine and further comprises N-trans-feruloyltyramine. In some embodiments, the N-trans-caffeoyltyramine and the N-trans-feruloyltyramine is in a ratio from about 10:1 to about 1:10. In some embodiments, the N-trans-caffeoyltyramine and the N-trans-feruloyltyramine is in a ratio from about 5:1 to about 1:5. In some embodiments, the N-trans-caffeoyltyramine and the N-trans-feruloyltyramine is in a ratio from about 5:1 to about 1:1. In some embodiments, the N-trans-caffeoyltyramine and the N-trans-feruloyltyramine is in a ratio from about 2.5:1 to about 2:1. In some embodiments, the N-trans-caffeoyltyramine and the N-trans-feruloyltyramine is in a ratio from about 2.2:1. In some embodiments, the food product is a food bar. In some embodiments, the food bar comprises from about 0.01% to about 20% (w/w) N-trans-caffeoyltyramine. In some embodiments, the food bar comprises from about 0.01% to about 20% (w/w) N-trans-feruloyltyramine. In some embodiments, the food product comprises at least 10% fiber. In some embodiments, the food product is a crisp. In some embodiments, the food bar comprises from about 0.01% to about 20% (w/w) N-trans-caffeoyltyramine. In some embodiments, the food bar comprises from about 0.01% to about 20% (w/w) N-trans-feruloyltyramine. In some embodiments, the food product comprises at least 10% fiber.

Some aspects relate to a method for improving transepithelial electrical resistance in a cell. In some embodiments, includes contacting the cell with a composition comprising at least one carrier and an effective amount of a compound of Formula (I),

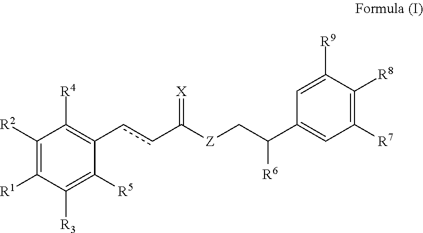

wherein
R¹, R², R³, R⁴, R⁵, R⁶, R⁷, R⁸, and R⁹ are each independently selected from hydrogen, deuterium, hydroxyl, halogen, cyano, nitro, optionally substituted amino, optionally substituted C-amido, optionally substituted N-amido, optionally substituted ester, optionally substituted —C₁₋₆alkyl, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl; the dashed bond is present or absent;
X is CH₂ or O;
Z is CHR^{a}, NR^{a}, or O; and
R^{a} is selected from hydrogen, deuterium, hydroxyl, halogen, cyano, nitro, optionally substituted amino, optionally substituted C-amido, optionally substituted N-amido, optionally substituted ester, optionally substituted —C₁₋₆alkyl, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl, thereby treating, improving or ameliorating a disease or condition associated with intestinal permeability in the subject.

In some embodiments, the compound of Formula (I) is selected from the group consisting of: N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, N-trans-chlorogenoyltyramine, N-trans-rosmarinoyltyramine, and 5-hydroxyferuloyltyramine, or a pharmaceutically acceptable salt, solvates, and combinations of the foregoing.

### BRIEF DESCRIPTION OF THE DRAWINGS

The features and advantages of the compositions and methods described herein will become apparent from the following description, taken in conjunction with the accompanying drawings. These drawings depict certain aspects of the compositions and methods described in the present application, and thus, are not to be considered limiting. In the drawings, similar reference numbers or symbols typically identify similar components, unless context dictates otherwise. The drawings may not be drawn to scale.

FIG. 1 illustrates four scatterplots (A-D) of TEER transverse colon monolayers in response to Compound 1 on 1.0 μm plates.

FIG. 2 illustrates four scatterplots (A-D) of TEER transverse colon monolayers in response to Compound 2 on 1.0 μm plates.

FIG. 3 illustrates four scatterplots (A-D) of TEER transverse colon monolayers in response to Compound 2-1 on 1.0 μm plates.

FIG. 4 illustrates four scatterplots (A-D) of TEER transverse colon monolayers in response to Compound 1 on 0.4 μm plates.

FIG. 5 illustrates four scatterplots (A-D) of TEER transverse colon monolayers in response to Compound 2 on 0.4 μm plates.

FIG. 6 illustrates four scatterplots (A-D) of TEER transverse colon monolayers in response to Compound 2-1 on 0.4 μm plates.

FIG. 7 illustrates four bar graphs (A-D) of permeability of transverse colon monolayers in response to Compound 1 on 1.0 μm plates.

FIG. 8 illustrates four bar graphs (A-D) of permeability of transverse colon monolayers in response to Compound 2 on 1.0 μm plates.

FIG. 9 illustrates four bar graphs (A-D) of permeability of transverse colon monolayers in response to Compound 2-1 on 1.0 μm plates.

FIG. 10 illustrates four bar graphs (A-D) of permeability of transverse colon monolayers in response to Compound 1 on 0.4 μm plates.

FIG. 11 illustrates four bar graphs (A-D) of permeability of transverse colon monolayers in response to Compound 2 on 0.4 μm plates.

FIG. 12 illustrates four bar graphs (A-D) of permeability of transverse colon monolayers in response to Compound 2-1 on 0.4 μm plates.

FIG. 13 illustrates two bar graphs from the administration of NCT protected against (a) decreased TEER and (b) increased permeability with TNF-induced inflammation. Compound 1 refers to NCT.

FIG. 14 illustrates two bar graphs from the administration of NFT protected against (a) decreased TEER and (b) increased permeability with TNF-induced inflammation. Compound 2 refers to NFT.

FIG. 15 illustrates administration of NCT: NFT (a) modulated TEER and (b) reduces permeability with TNF-induced inflammation. Compound 2-1 refers to a combination of NCT and NFT.

### DETAILED DESCRIPTION

This disclosure provides, among other things, the discovery of strong HNF4α expression enhancers and methods of improving gut permeability and improvement of impaired gut barrier function. In aspects, formulations and compositions containing one or more tyramine containing hydroxycinnamic acid amides are provided herein. Some embodiments provided herein provide for the compositions for the use in methods of treating, improving or ameliorating a disease or condition associated with intestinal permeability or improving gut barrier function in a subject. Some embodiments provided herein provide for the compositions for the use in methods for improving transepithelial electrical resistance in a cell.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

The terms “effective amount” or a “therapeutically effective amount” as used herein refers to an amount of a therapeutic agent that is effective to relieve, to some extent, or to reduce the likelihood of onset of, one or more of the symptoms of a disease or condition, and can include curing a disease or condition.

An “extract” refers to a composition containing one or more compounds described herein, which is separated from other unwanted substances present in the natural source material from which the extract was obtained. In some embodiments, the natural source material is a plant. Plant extracts can be obtained from any plant tissue including a whole plant; a plant part such as shoot vegetative organs/structures (for example, leaves, stems and tubers), roots, flowers and floral organs/structures (for example, bracts, sepals, petals, stamens, carpels, anthers and ovules), a seed (including embryo, endosperm, and seed coat) or fruit (the mature ovary); a plant tissue (for example, vascular tissue, ground tissue, and the like); cells (for example, guard cells, egg cells, and the like), or exudates as well as progeny and cultures or cell lines of the same. Preferably, the extract contains compounds that will be found to be generally recognized as safe (GRAS) for human consumption. Accordingly, in certain embodiments the extract is from an edible source. In this respect, the extract is an edible extract.

As used herein, “immunomodulation”, “immunomodulatory”, and “immunostimulatory” mean causing, or having the capacity to cause, a detectable change in an immune response, and the ability to cause a detectable change in an immune response. The terms “immunomodulation”, “immunomodulatory”, and “immunostimulatory” as used herein includes immunostimulatory as well as immunosuppressive effects. Immunomodulation is primarily a qualitative alteration in an overall immune response, although quantitative changes may also occur in conjunction with immunomodulation.

The terms “pharmaceutically acceptable” as used herein is a broad term, and is to be given its ordinary and customary meaning to a person of ordinary skill in the art (and is not to be limited to a special or customized meaning), and refers without limitation to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable risk/benefit ratio.

The terms “pharmaceutically acceptable salts” and “a pharmaceutically acceptable salt thereof as used herein are broad terms, and are to be given their ordinary and customary meaning to a person of ordinary skill in the art (and is not to be limited to a special or customized meaning), and refer without limitation to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of the therapeutic agents discussed herein, provided that it is non-toxic and does not substantially interfere with the desired activity. In addition to salts, pharmaceutically acceptable precursors and derivatives of the compounds can be employed. Pharmaceutically acceptable amides, lower alkyl derivatives, and protected derivatives can also be suitable for use in compositions and methods of preferred embodiments. While it may be possible to administer the compounds of the preferred embodiments in the form of pharmaceutically acceptable salts, it is generally preferred to administer the compounds in neutral form.

The term “pharmaceutical composition” as used herein is a broad term, and is to be given its ordinary and customary meaning to a person of ordinary skill in the art (and is not to be limited to a special or customized meaning), and refers without limitation to a mixture of one or more compounds disclosed herein with other chemical components, such as diluents or carriers. The pharmaceutical composition facilitates administration of the compound to an organism. Pharmaceutical compositions can also be obtained by reacting compounds with inorganic or organic acids or bases. Pharmaceutical compositions will generally be tailored to the specific intended route of administration.

As used herein, a “carrier” as used herein is a broad term, and is to be given its ordinary and customary meaning to a person of ordinary skill in the art (and is not to be limited to a special or customized meaning), and refers without limitation to a compound that facilitates the incorporation of a compound into cells or tissues. A carrier means a material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. For example, without limitation, dimethyl sulfoxide (DMSO) is a commonly utilized carrier that facilitates the uptake of many organic compounds into cells or tissues of a subject. Water, saline solution, ethanol, and mineral oil are also carriers employed in certain pharmaceutical compositions.

As used herein, a “diluent” is a broad term, and is to be given its ordinary and customary meaning to a person of ordinary skill in the art (and is not to be limited to a special or customized meaning), and refers without limitation to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, the term “food product” means any food, feed, snack, food supplement, treat, meal substitute, or meal replacement, whether intended for a human or an animal. Animal food includes food or feed intended for any domesticated or wild species. In preferred embodiments, a food for an animal represents a nutritionally complete food or dietary composition. Examples of such animal foods include extruded pet foods such as foods for canines and felines, e.g., dogs and cats.

As used herein, “isomer” refers to especially optical isomers (for example essentially pure enantiomers, essentially pure diastereomers, and mixtures thereof) as well as conformation isomers (i.e., isomers that differ only in their angles of at least one chemical bond), position isomers (particularly tautomers), and geometric isomers (e.g., cis-trans isomers).

As used herein, an “excipient” as used herein is a broad term, and is to be given its ordinary and customary meaning to a person of ordinary skill in the art (and is not to be limited to a special or customized meaning), and refers without limitation to a substance that is added to a pharmaceutical composition to provide, without limitation, bulk, consistency, stability, binding ability, lubrication, disintegrating ability etc., to the composition. A “diluent” is a type of excipient.

As used herein, a “subject” as used herein is a broad term, and is to be given its ordinary and customary meaning to a person of ordinary skill in the art (and is not to be limited to a special or customized meaning), and refers without limitation to an animal that is the object of treatment, observation or experiment. “Animal” includes cold- and warm-blooded vertebrates and invertebrates such as fish, shellfish, reptiles and, in particular, mammals. “Mammal” includes, without limitation, dolphins, mice, rats, rabbits, guinea pigs, dogs, cats, sheep, goats, cows, horses, primates, such as monkeys, chimpanzees, and apes, and, in particular, humans. In some embodiments, the subject is human.

As used herein, the terms “treating,” “treatment,” “therapeutic,” or “therapy” are broad terms, and are to be given their ordinary and customary meaning (and are not to be limited to a special or customized meaning) and, without limitation, do not necessarily mean total cure or abolition of the disease or condition. Any alleviation of any undesired markers, signs or symptoms of a disease or condition, to any extent, can be considered treatment and/or therapy. Furthermore, treatment may include acts that may worsen the patient's overall feeling of well-being or appearance.

The term “ameliorate” as used herein refers to any reduction in the extent, severity, frequency, and/or likelihood of a symptom or clinical sign characteristic of a particular condition.

The terms “therapeutically effective amount” and “effective amount” as used herein are broad terms, and are to be given its ordinary and customary meaning to a person of ordinary skill in the art (and are not to be limited to a special or customized meaning), and are used without limitation to indicate an amount of an active compound, or pharmaceutical agent, that elicits the biological or medicinal response indicated. For example, a therapeutically effective amount of compound can be the amount needed to prevent, alleviate or ameliorate markers or symptoms of a condition or prolong the survival of the subject being treated. This response may occur in a tissue, system, animal or human and includes alleviation of the signs or symptoms of the disease being treated. Determination of a therapeutically effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

The term “solvents” as used herein is a broad term, and is to be given its ordinary and customary meaning to a person of ordinary skill in the art (and is not to be limited to a special or customized meaning), and refers without limitation to compounds with some characteristics of solvency for other compounds or means, that can be polar or nonpolar, linear or branched, cyclic or aliphatic, aromatic, naphthenic and that includes but is not limited to: alcohols, derivatives, diesters, ketones, acetates, terpenes, sulfoxides, glycols, paraffins, hydrocarbons, anhydrides, heterocyclics, among others.

As used herein, a “nutraceutical” generally refers to natural, bioactive chemical compounds that provide physiological benefits, including disease prevention and health promotion which may be used to supplement the diet. Nutraceuticals can be either purified or concentrated by using bioengineering methods and can be enhanced through genetic methods, which contain elevated levels of natural substances. Examples of nutraceuticals include isolated nutrients and herbal products and generally contain at least one of the following ingredients: a vitamin, a mineral, an herb or other botanical, an amino acid, a metabolite, constituent, extract, or combination of these ingredients. Common examples of nutraceuticals include beta-carotene, ephedra, ginko biloba, goldenseal, valerian, ginseng, green tea extract, and echinacea. The nutraceuticals described herein may be useful for maintenance and support of, for example, healthy joints, skin, eye and brain function, heart and circulatory system, and general health.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, and hexyls. The alkyl group may be substituted or unsubstituted.

The term “halogen atom” or “halogen” as used herein, means any one of the radio-stable atoms of column 7 of the Periodic Table of the Elements, such as chloro (Cl), fluoro (F), bromo (Br) and iodo (I) groups.

In any of the groups described herein, an available hydrogen may be replaced with an alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, alkoxyalkoxy, alkoxycarbonyl, acyl, halo, nitro, aryloxycarbonyl, cyano, carboxy, aralkoxycarbonyl, alkylsulfonyl, arylsulfonyl, heteroarylsulfonyl, alkylthio, arylthio, heteroarylthio, aralkylthio, heteroaralkylthio, cycloalkyl, or heterocyclyl.

Any undefined valency on an atom of a structure shown in this application implicitly represents a hydrogen atom bonded to the atom.

As used herein, “alkenyl” refers to an alkyl group, as defined herein, that contains in the straight or branched hydrocarbon chain one or more double bonds. An alkenyl group may be unsubstituted or substituted.

As used herein, “alkynyl” refers to an alkyl group as defined herein, that contains in the straight or branched hydrocarbon chain one or more triple bonds. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl, and cyclooctyl.

As used herein, “aryl” refers to a carbocyclic (all carbon) monocyclic or multicyclic aromatic ring system (including, e.g., fused, bridged, or spiro ring systems where two carbocyclic rings share a chemical bond, e.g., one or more aryl rings with one or more aryl or non-aryl rings) that has a fully delocalized pi-electron system throughout at least one of the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene, and azulene. An aryl group may be substituted or unsubstituted.

As used herein, “heterocyclyl” refers to mono- or polycyclic ring systems including at least one heteroatom (e.g., O, N, S). Such systems can be unsaturated, can include some unsaturation, or can contain some aromatic portion, or be all aromatic. A heterocyclyl group can contain from 3 to 30 atoms. A heterocyclyl group may be unsubstituted or substituted.

As used herein, “heteroaryl” refers to a monocyclic or multicyclic aromatic ring system (a ring system having a least one ring with a fully delocalized pi-electron system) that contain(s) one or more heteroatoms, that is, an element other than carbon, including but not limited to, nitrogen, oxygen, and sulfur, and at least one aromatic ring. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline, and triazine. A heteroaryl group may be substituted or unsubstituted.

The term “amino” as used herein refers to a —NH₂ group.

As used herein, the term “hydroxy” refers to a —OH group.

A “cyano” group refers to a “—CN” group.

A “carbonyl” group refers to a C═O group.

A “C-amido” group refers to a “—C(═O)N(R_{A}R_{B})” group in which R_{A} and R_{B} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined above. A C-amido may be substituted or unsubstituted.

An “N-amido” group refers to a “RC(═O)N(R_{A})—” group in which R and R_{A} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined above. An N-amido may be substituted or unsubstituted.

A “urea” group refers to a “—N(R_{A}R_{B})—C(═O)—N(R_{A}R_{B})—” group in which R_{A} and R_{B} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined above. A urea group may be substituted or unsubstituted.

For the groups herein, the following parenthetical subscripts further define the groups as follows: “(Cₙ)” defines the exact number (n) of carbon atoms in the group. For example, “C₁-C₆-alkyl” designates those alkyl groups having from 1 to 6 carbon atoms (e.g., 1, 2, 3, 4, 5, or 6, or any range derivable therein (e.g., 3-6 carbon atoms)).

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value. Where particular values are described in the application and claims, unless otherwise stated the term “about” meaning within an acceptable error range for the particular value should be assumed.

As used herein, the term “weight percent,” when referring to a component, is the weight of the component divided by the weight of the composition that includes the component, multiplied by 100%. For example, the weight percent of component A when 5 grams of component A is added to 95 grams of component B is 5% (e.g., 5 g A/(5 g A+95 g B)×100%).

Any percentages, ratios or other quantities referred to herein are on a weight basis, unless otherwise indicated.

### Compositions

In aspects, disclosed herein are compositions comprising one or more tyramine containing hydroxycinnamic acid amides and their use in treating a gastrointestinal disorder or improving a digestive function. In some embodiments, the one or more tyramine containing hydroxycinnamic acid amides are plant-derived aromatic metabolites with one or more acidic hydroxyl groups attached to aromatic arenes. In some embodiments, the one or more tyramine containing hydroxycinnamic acid amides are derived from a cell. In some embodiments, the one or more tyramine containing hydroxycinnamic acid amides are synthetically produced.

In particular, the disclosure encompasses a compound of Formula (I), or an isomer, salt, homodimer, heterodimer, or conjugate thereof:

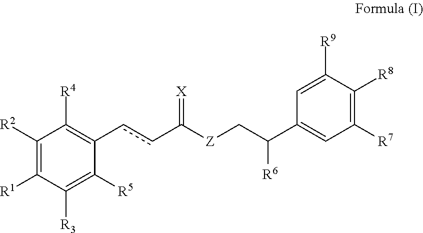

In some embodiments, the composition includes a compound of Formula (II), or an isomer, salt, homodimer, heterodimer, or conjugate thereof:

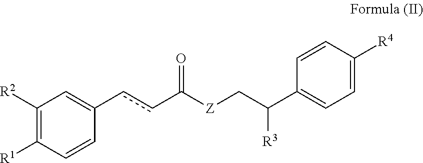

In some embodiments of the compounds of Formula (I), R¹, R², R³, R⁴, R⁵, R⁶, R⁷, R⁸, and R⁹ are each independently selected from hydrogen, deuterium, hydroxyl, halogen, cyano, nitro, optionally substituted amino, optionally substituted C-amido, optionally substituted N-amido, optionally substituted ester, optionally substituted —C₁₋₆alkyl, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆ alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

In some embodiments of the compounds of Formula (I), R¹, R², R³, and R⁸ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl, and R⁴, R⁵, R⁶, R⁷, and R⁹ are each independently hydrogen, deuterium, hydroxyl, or halogen;

In some embodiments of Formula (I), R¹, R², and R⁸ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆ alkylC₁₋₁₂heteroaryl, and R³, R⁴, R⁵, R⁶, R⁷, and R⁹ are each independently hydrogen, deuterium, hydroxyl, or halogen.

In some embodiments of the compound of Formula (II), R¹, R², R³, and R⁴ are each independently selected from hydrogen, deuterium, hydroxyl, halogen, cyano, nitro, optionally substituted amino, optionally substituted C-amido, optionally substituted N-amido, optionally substituted ester, optionally substituted —C₁₋₆alkyl, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

In some embodiments of the compounds of Formula (I) or Formula (II), the dashed bond is present or absent.

In some embodiments of the compounds of Formula (I), X is CH₂ or O.

In some embodiments of the compounds of Formula (I) or Formula (II), Z is CHR^{a}, NR^{a}, or O.

In some embodiments of the compounds of Formula (I) or Formula (II), R^{a} is selected from hydrogen, deuterium, hydroxyl, halogen, cyano, nitro, optionally substituted amino, optionally substituted C-amido, optionally substituted N-amido, optionally substituted ester, optionally substituted —C₁₋₆alkyl, optionally substituted —(O)C₁₋₆alkyl, optionally substituted —(O)C₁₋₆alkenyl, optionally substituted —(O)C₁₋₆ alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆aylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

In some embodiments, a compound of Formula (I) or Formula (II) is provided as a pharmaceutically acceptable salt or solvate thereof.

In some embodiments, a compound of Formula (I) or Formula (II) is selected from (E)-3-(3,4-dihydroxyphenyl)-N-(4-ethoxyphenethyl)acrylamide, (E)-3-(3,4-dihydroxyphenyl)-N-(4-(2-methoxyethoxy)phenethyl)acrylamide, (E)-3-(3,4-dihydroxyphenyl)-N-(4-(2-(methylsulfonyl)ethoxy)phenethyl)acrylamide, (E)-2-(4-(2-(3-(3,4-dihydroxyphenyl)acrylamido)ethyl)phenoxy)acetic acid, ethyl (E)-2-(4-(2-(3-(3,4-dihydroxyphenyl)acrylamido)ethyl)phenoxy)acetate, (E)-N-(4-(cyclopropylmethoxy)phenethyl)-3-(3,4-dihydroxyphenyl)acrylamide, (E)-3-(3,4-dihydroxyphenyl)-N-(4-(3,3,3-trifluoropropoxy)phenethyl)acrylamide, (E)-3-(3,4-dihydroxyphenyl)-N-(4-((tetrahydro-2H-pyran-4-yl)methoxy)phenethyl)acrylamide, (E)-3-(3,4-dihydroxyphenyl)-N-(4-((4-fluorobenzyl)oxy)phenethyl)acrylamide, (E)-N-(4-(cyanomethoxy)phenethyl)-3-(3,4-dihydroxyphenyl)acrylamide, (E)-3-(3,4-dihydroxyphenyl)-N-(4-(pyridin-3-ylmethoxy)phenethyl)acrylamide, (E)-3-(3,4-dihydroxyphenyl)-N-(4-(pyridin-2-ylmethoxy)phenethyl)acrylamide, (E)-3-(3,4-dihydroxyphenyl)-N-(4-(2-(dimethylamino)ethoxy)phenethyl)acrylamide, (E)-3-(3,4-dihydroxyphenyl)-N-(4-isobutoxyphenethyl)acrylamide, (E)-3-(3,4-dihydroxyphenyl)-N-(4-(pyridin-4-ylmethoxy)phenethyl)acrylamide, (E)-3-(3,4-dihydroxyphenyl)-N-(4-((4-methoxybenzyl)oxy)phenethyl)acrylamide, (E)-3-(3,4-dihydroxyphenyl)-N-(4-(oxetan-3-ylmethoxy)phenethyl)acrylamide, (E)-3-(3,4-dihydroxyphenyl)-N-(4-((tetrahydro-2H-pyran-2-yl)methoxy)phenethyl)acrylamide, (E)-3-(3,4-dihydroxyphenyl)-N-(4-((tetrahydrofuran-2-yl)methoxy)phenethyl)acrylamide, (E)-3-(3,4-dihydroxyphenyl)-N-(4-(thiophen-2-yloxy)phenethyl)acrylamide, (E)-3-(3,4-dihydroxyphenyl)-N-(4-(3,3-dimethylbutoxy)phenethyl)acrylamide, (E)-3-(3,4-dihydroxyphenyl)-N-(4-(2-hydroxyethoxy)phenethyl)acrylamide, (E)-N-(4-((1H-tetrazol-5-yl)methoxy)phenethyl)-3-(3,4-dihydroxyphenyl)acrylamide, (E)-3-(3,4-dihydroxyphenyl)-N-(4-((1-methylpyrrolidin-2-yl)methoxy)phenethyl)acrylamide, (E)-2-hydroxy-5-(3-((4-hydroxyphenethyl)amino)-3-oxoprop-1-en-1-yl)phenyl hydrogen carbonate, (E)-3-(4-hydroxy-3-(pyridin-4-yloxy)phenyl)-N-(4-hydroxyphenethyl)acrylamide, (E)-3-(4-hydroxy-3-isobutoxyphenyl)-N-(4-hydroxyphenethyl)acrylamide, (E)-3-(3-(4-fluorophenoxy)-4-hydroxyphenyl)-N-(4-hydroxyphenethyl)acrylamide, (E)-3-(3-(cyanomethoxy)-4-hydroxyphenyl)-N-(4-hydroxyphenethyl)acrylamide, (E)-2-(2-hydroxy-4-(3-((4-hydroxyphenethyl)amino)-3-oxoprop-1-en-1-yl)phenoxy)acetic acid, (E)-3-(3-hydroxy-4-(pyridin-4-ylmethoxy)phenyl)-N-(4-hydroxyphenethyl)acrylamide, (E)-3-(4-((4-fluorobenzyl)oxy)-3-hydroxyphenyl)-N-(4-hydroxyphenethyl)acrylamide, (E)-3-(3-hydroxy-4-isobutoxyphenyl)-N-(4-hydroxyphenethyl)acrylamide, (E)-3-(4-(cyanomethoxy)-3-hydroxyphenyl)-N-(4-hydroxyphenethyl)acrylamide, (E)-N-(3-(3,4-dihydroxyphenyl)acryloyl)-N-(4-hydroxyphenethyl)glycine, (E)-3-(3,4-dihydroxyphenyl)-N-(4-hydroxyphenethyl)-N-(pyridin-4-ylmethyl)acrylamide, (E)-3-(3,4-dihydroxyphenyl)-N-(4-hydroxyphenethyl)-N-isobutylacrylamide, (E)-N-(cyanomethyl)-3-(3,4-dihydroxyphenyl)-N-(4-hydroxyphenethyl)acrylamide, 3-(3,4-dihydroxyphenyl)-N-(4-hydroxyphenethyl)propanamide, 3-(3,4-dihydroxyphenyl)-N-(4-(methylsulfonamido)phenethyl)propanamide, or pharmaceutical salts, solvates, and combination of the foregoing.

In certain embodiments, a compound of Formula (I) or Formula (II) is selected from:

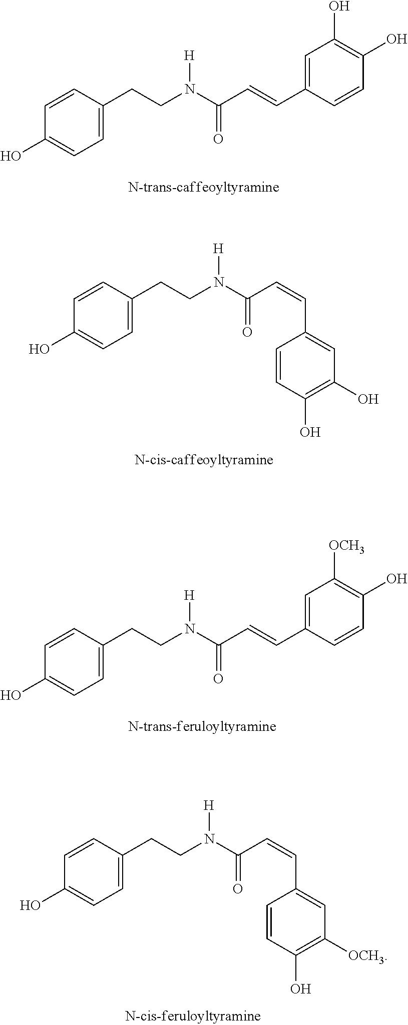 [SMILES: O=C(/C=C/c1ccc(O)c(O)c1)NCCc1ccc(O)cc1 ; O=C(/C=C\c1ccc(O)c(O)c1)NCCc1ccc(O)cc1 ; COc1cc(/C=C/C(=O)NCCc2ccc(O)cc2)ccc1O ; COc1cc(/C=C\C(=O)NCCc2ccc(O)cc2)ccc1O]

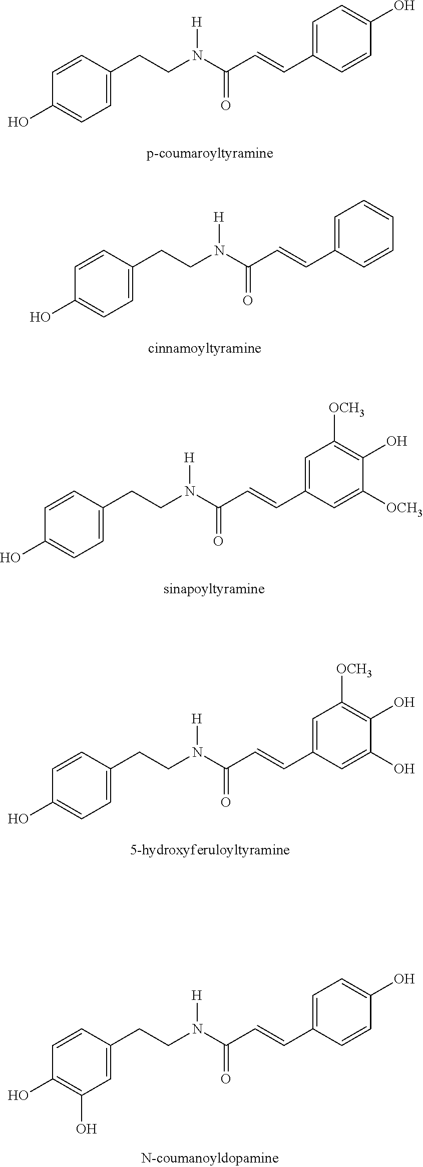 [SMILES: O=C(/C=C/c1ccc(O)cc1)NCCc1ccc(O)cc1 ; O=C(/C=C/c1ccccc1)NCCc1ccc(O)cc1 ; COc1cc(/C=C/C(=O)NCCc2ccc(O)cc2)cc(OC)c1O ; COc1cc(/C=C/C(=O)NCCc2ccc(O)cc2)cc(O)c1O ; O=C(/C=C/c1ccc(O)cc1)NCCc1ccc(O)c(O)c1]

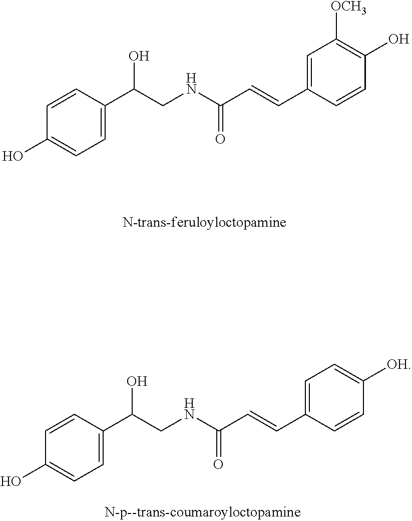 [SMILES: COc1cc(/C=C/C(=O)NCC(O)c2ccc(O)cc2)ccc1O ; O=C(/C=C/c1ccc(O)cc1)NCC(O)c1ccc(O)cc1]

A salt of a compound of this disclosure refers to a compound that possesses the desired pharmacological activity of the parent compound and includes: (1) an acid addition salt, formed with an inorganic acid such as hydrochloric acid, hydrobromic acid, sulfuric acid, nitric acid, phosphoric acid, and the like; or formed with an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic camphorsulfonic acid, acid, 4-toluenesulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) a salt formed when an acidic proton present in the parent compound is replaced.

As is known in the art, a homodimer is a molecule composed of two identical tyramine containing hydroxycinnamic acid amide subunits. By comparison, a heterodimer is a molecule composed of two different tyramine containing hydroxycinnamic acid amide subunits. Examples of homodimers of this disclosure include but are not limited to a cross-linked N-trans-feruloyltyramine dimer, a cross-linked N-trans-caffeoyl tyramine dimer and a cross-linked p-coumaroyltyramine dimer. See, for example, King & Calhoun (2005) Phytochemistry 66(20): 2468-73, which teaches the isolation of a cross-linked N-transferuloyltyramine dimer from potato common scab lesions.

Conjugates of monomers of tyramine containing hydroxycinnamic acid amide and other compounds, such as lignan amides. Examples of conjugates include, but are not limited to cannabisin A, cannabisin B, cannabisin C, cannabisin D, cannabisin E, cannabisin F and grossamide.

Whenever a group is described as being “optionally substituted” that group may be unsubstituted or substituted with one or more of the indicated substituents. Likewise, when a group is described as being “unsubstituted or substituted” if substituted, the substituent may be selected from one or more the indicated substituents. If no substituents are indicated, it is meant that the indicated “optionally substituted” or “substituted” group may be individually and independently substituted with one or more group(s) individually and independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, mercapto, alkylthio, arylthio, cyano, halogen, thiocarbonyl, 0-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, amino, mono-substituted amino group and di-substituted amino group, and protected derivatives thereof.

In addition to isomers, salts, homodimers, heterodimers, and conjugates, the tyramine containing hydroxycinnamic acid amide may also be glycosylated. A glycosylated tyramine containing hydroxycinnamic acid amide may be produced by transglycosylating the tyramine containing hydroxycinnamic acid amide to add glucose units, for example, one, two, three, four, five, or more than five glucose units, to the tyramine containing hydroxycinnamic acid amide. Transglycosylation can be carried out with any suitable enzyme including, but not limited to, a pullulanase and isomaltase (Lobov, et al. (1991) Agric. Biol. Chem. 55:2959-2965), ˜-galactosidase (Kitahata, et al. (1989) Agric. Biol. Chem. 53:2923-2928), dextrine saccharase (Yamamoto, et al. (1994) Biosci. Biotech. Biochem. 58: 1657-1661) or cyclodextrin gluconotransferase, with pullulan, maltose, lactose, partially hydrolyzed starch and maltodextrin being donors.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, or may be stereoisomeric mixtures, and include all diastereomeric, and enantiomeric forms. In addition it is understood that, in any compound described herein having one or more double bond(s) generating geometrical isomers that can be defined as E or Z, each double bond may independently be E or Z a mixture thereof. Stereoisomers are obtained, if desired, by methods such as, stereoselective synthesis and/or the separation of stereoisomers by chiral chromatographic columns.

Likewise, it is understood that, in any compound described, all tautomeric forms are also intended to be included.

It is understood that the compounds described herein can be labeled isotopically or by another other means, including, but not limited to, the use of chromophores or fluorescent moieties, bioluminescent labels, or chemiluminescent labels. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium), hydrogen-2 (deuterium), and hydrogen-3 (tritium). Thus, reference herein to a compound encompasses all potential isotopic forms unless the context clearly dictates otherwise.

It is understood that the compounds described herein can be labeled isotopically or by another other means, including, but not limited to, the use of chromophores or fluorescent moieties, bioluminescent labels, or chemiluminescent labels. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium), hydrogen-2 (deuterium), and hydrogen-3 (tritium). Thus, reference herein to a compound encompasses all potential isotopic forms unless the context clearly dictates otherwise.

It is understood that the methods and formulations described herein include the use of crystalline forms, amorphous phases, and/or pharmaceutically acceptable salts, solvates, hydrates, and conformers of compounds of some embodiments, as well as metabolites and active metabolites of these compounds having the same type of activity. A conformer is a structure that is a conformational isomer. Conformational isomerism is the phenomenon of molecules with the same structural formula but different conformations (conformers) of atoms about a rotating bond. In specific embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein. Other forms in which the compounds of some embodiments can be provided include amorphous forms, milled forms and nano-particulate forms.

Likewise, it is understood that the compounds described herein, such as compounds of some embodiments, include the compound in any of the forms described herein (e.g., pharmaceutically acceptable salts, prodrugs, crystalline forms, amorphous form, solvated forms, enantiomeric forms, tautomeric forms, and the like).

In some embodiments, the composition as described herein may include one or more compounds of Formula (I) or Formula (II). In some embodiments, the one or more compounds described herein may be in a ratio from about 10:1 to about 1:10, or ranges including and/or spanning the aforementioned values. In some embodiments, the one or more compounds described herein may be in a ratio from about 10:1, 9.5:1, 8.5:1, 8.0:1, 7.5:1, 7:1, 6.5:1, 6:1, 5.5:1, 5:1, 4.5:1, 4:1, 3.5:1, 3.0:1, 2.5:1, 2.0:1, 1.5:1, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, 1:10. In some embodiments, the one or more compounds may be selected from N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, and 5-hydroxyferuloyltyramine, a salt, or isomer thereof. In some embodiments, the one or more compounds is N-trans-caffeoyltyramine. In some embodiments, the one or more compounds is N-trans-feruloyltyramine. In some embodiments, the one or more compounds includes N-trans-caffeoyltyramine and N-trans-feruloyltyramine. In some embodiments, the ratio of N-trans-caffeoyltyramine to N-trans-feruloyltyramine is from about 10:1 to about 1:10. In some embodiments, the ratio of N-trans-caffeoyltyramine to N-trans-feruloyltyramine is from about 5:1 to about 1:5. In some embodiments, the N-trans-caffeoyltyramine and the N-trans-feruloyltyramine is in a ratio from about 2.5:1 to about 2:1. In some embodiments, the ratio of N-trans-caffeoyltyramine to N-trans-feruloyltyramine is from about 2.5:1 to about 1:2.5. In some embodiments, the ratio of N-trans-caffeoyltyramine to N-trans-feruloyltyramine is from about 5:1. In some embodiments, the ratio of N-trans-caffeoyltyramine to N-trans-feruloyltyramine is from about 4:1. In some embodiments, the ratio of N-trans-caffeoyltyramine to N-trans-feruloyltyramine is from about 3:1. In some embodiments, the ratio of N-trans-caffeoyltyramine to N-trans-feruloyltyramine is from about 2.2:1. In some embodiments, the ratio of N-trans-caffeoyltyramine to N-trans-feruloyltyramine is from about 2:1. In some embodiments, the ratio of N-trans-caffeoyltyramine to N-trans-feruloyltyramine is from about 1:2. In some embodiments, the ratio of N-trans-caffeoyltyramine to N-trans-feruloyltyramine is from about 1:2.2. In some embodiments, the ratio of N-trans-caffeoyltyramine to N-trans-feruloyltyramine is from about 1:3. In some embodiments, the ratio of N-trans-caffeoyltyramine to N-trans-feruloyltyramine is from about 1:4. In some embodiments, the ratio of N-trans-caffeoyltyramine to N-trans-feruloyltyramine is from about 1:5.

In some embodiments, the one or more compounds is from an extract of a plant or a plant source. In some embodiments, the extract includes one or more compounds as described herein. In some embodiments, the extract of the plant is selected from the group comprising Allium, Amoracia, Chenopodium, Cannabis, Spinacia, Fagopyrum, Annona, Jatropha, Piper, Eragrostis, Zea, Nelumbo, Ipomoea, Capsicum, Lycium, Solanum, and Tribulus. In some embodiments, the extract of the plant is hemp hulls, peppercorn, or black seed. In some embodiments, the extract is from hemp hulls.

### Formulations

Aspects of the disclosure relate to formulation including one or more compounds as described herein. In some embodiments, a substantially pure compound or extract comprising one or more compounds of this disclosure can be combined with a carrier and provided in any suitable form for consumption by, provided to, or administration to a subject. In some embodiments, the one or more compounds or extract comprising one or more compounds is added as an exogenous ingredient or additive to a consumable. Suitable consumable forms include, but are not limited to, a dietary supplement, food ingredient or additive, a food product, a medical food, nutraceutical or pharmaceutical composition. In some embodiments, the one or more compounds or extract comprising one or more compounds is provided in either a liquid, granular, or powder form. In some embodiments, the formulation may be a food product.

A food ingredient or additive includes an edible substance intended to result, directly or indirectly, in its becoming a component or otherwise affecting the characteristic of any food (including any substance intended for use in producing, manufacturing, packing, processing, preparing, treating, packaging, transporting, or holding food). A food product, in particular a functional food, includes a food fortified or enriched during processing to include additional complementary nutrients and/or beneficial ingredients. A food product according to this disclosure can, e.g., be in the form of butter, margarine, sweet or savory spreads, condiment, biscuits, food bar, crisp, cracker, granola, health bar, bread, cake, cereal, candy, confectionery, soup, milk, yogurt or a fermented milk product, cheese, juice-based and vegetable-based beverages, functional beverage, powdered beverage mix, fermented beverages, shakes, flavored waters, tea, oil, or any other suitable food. In some embodiments, the food product includes a whole-food product in which the concentration of the one or more compounds have been enriched through particular post-harvest and food production processing methods to levels that provide an efficacious amount of the compound. In some embodiments, the food product includes one or more compounds as described herein and a fiber source. In some embodiments, the food product includes a compound selected from the group consisting of: N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, N-trans-chlorogenoyltyramine, N-trans-rosmarinoyltyramine, and 5-hydroxyferuloyltyramine, or a pharmaceutically acceptable salt, solvates, and combinations of the foregoing. In some embodiments, the food product includes N-trans-feruloyltyramine. In some embodiments, the food product includes N-trans-caffeoyltyramine. In some embodiments, the food product includes N-trans-caffeoyltyramine and N-trans-feruloyltyramine. In some embodiments, the N-trans-caffeoyltyramine and the N-trans-feruloyltyramine are in a ratio from about 10:1 to about 1:10. In some embodiments, the N-trans-caffeoyltyramine and the N-trans-feruloyltyramine are in a ratio from about 5:1 to about 1:5. In some embodiments, the N-trans-caffeoyltyramine and the N-trans-feruloyltyramine are in a ratio from about 5:1 to about 1:1. In some embodiments, the N-trans-caffeoyltyramine and the N-trans-feruloyltyramine is in a ratio from about 2.5:1 to about 2:1. In some embodiments, the N-trans-caffeoyltyramine and the N-trans-feruloyltyramine are in a ratio at about 2.2:1. In some embodiments, the food product is a food bar. In some embodiments, the food bar comprises from about 0.01% to about 20% (w/w) N-trans-caffeoyltyramine. In some embodiments, the food bar comprises from about 0.01% to about 20% (w/w) N-trans-feruloyltyramine. In some embodiments, the food product is a crisp. In some embodiments, the food bar comprises from about 0.01% to about 20% (w/w) N-trans-caffeoyltyramine. In some embodiments, the food bar comprises from about 0.01% to about 20% (w/w) N-trans-feruloyltyramine.

In some embodiments, food product includes a carbohydrate. A variety of carbohydrates are used in food products, such as various sugars and starches. Carbohydrates are an important source of energy for the body, including complex carbohydrates (like whole grains and vegetables) and simple carbohydrates (like sugar and refined grains). There are several common carbohydrates that are used in food products, including: starch, sugar, fructose, maltodextrin, dextrose, corn syrup, oligosaccharides, cellulose, complex carbohydrates, such as inulin that may be added as a prebiotic fiber in processed foods. According to the disclosure, the concentration of carbohydrates may vary depending on the intended use of a product. In some embodiments, carbohydrates may comprise from about 1% by weight to about 95% by weight of a food product. In some embodiments, carbohydrates may comprise from about 1% by weight to about 95% by weight of a food product. In some embodiments, a food product may comprise carbohydrates in a range with high and low values independently selected from about 1%, 3%, 5%, 8%, 10%, 15%, 20%, 25%, 30%, 35%, 40%, 45%, 50%, 55%, 60%, 65%, 70%, 75%, 80%, 85%, 90%, 95% or ranges including, between, and/or spanning the aforementioned values.

In some embodiments, the food product may include a sugar. Sugars, such as high fructose corn syrup, sucrose, glucose syrup, dextrose, and other sweeteners are often added to processed foods to make them more palatable. Additionally, sugar alcohols or maltodextrins, may be added to food products to reduce caloric content and/or to enhance nutritional profile of the product. In some embodiments, a food product may comprise one or a plurality of saccharides that are slowly or incompletely digested by humans, if not totally indigestible. These sugars can include isomaltose, panose and branched oligomers having a degree of polymerization of four or greater. Additional non-limiting examples of sugars include sucrose, HFCS, fructose, brown sugar (which can be either partially or fully refined), powdered sugar (also known as confectioner's sugar), high fructose corn syrup, honey, molasses, maple syrup, agave nectar, coconut sugar, date sugar, fruit juice concentrates, maltodextrin, dextrose, glucose syrup, maple syrup, molasses, and lactose. According to the disclosure, the concentration of sugars may vary depending on the intended use of a product. In some embodiments, sugar may comprise from about 1% by weight to about 95% by weight of a food product. In some embodiments, the sugar(s) component comprises from about 2% to about 10% by weight of the composition. In some embodiments, sugars may comprise from about 1% by weight to about 95% by weight of a food product. In some embodiments, a food product may comprise sugars in a range with high and low values independently selected from about 1%, 3%, 5%, 8%, 10%, 15%, 20%, 25%, 30%, 35%, 40%, 45%, 50%, 55%, 60%, 65%, 70%, 75%, 80%, 85%, 90%, 95% or ranges including, between, and/or spanning the aforementioned values.

In some embodiments, the food product includes a fiber source. In some embodiments, the food product includes from about 5% to about 30% (w/w) fiber. In some embodiments, the food product includes at least 10% fiber. In some embodiments, the food product includes at least 15% fiber. In some embodiments, the food product includes at least 20% fiber. In some embodiments, the food product includes at least 30% fiber.

In some embodiments, the food product may include a protein. Proteins may be included in a range of concentrations depending on the product. Protein sources can include soy protein, soy flour, soy protein isolate, whey protein isolate, casein, gelatin, legume protein isolates, soy protein concentrate, egg albumin or egg white, wheat protein concentrate, legume protein concentrates and mixtures thereof. In some embodiments, meat products like beef jerky, sausages, or meatballs, may include proteins as a primary ingredient. Proteins may be included at concentrations ranging from about 10% to about 25% of the total ingredients. In dairy products like cheese, yogurt, or milk, proteins like casein or whey may be the primary ingredient. Proteins may be included at concentrations ranging from about 3% to about 15% of the total ingredients. In plant-based products like tofu, tempeh, or seitan, proteins from soy, peas, legumes, or wheat may be primary ingredient. Proteins may be included at concentrations ranging from about 10% to about 25% of the total ingredients. In energy bars and protein bars, proteins like whey or soy, or any protein according to the disclosure may be added to provide a source of protein. Proteins may be included at concentrations ranging from about 10% to about 25% of the total ingredients. According to the disclosure, the concentration of proteins may vary depending on the intended use of a product. In some embodiments, proteins may comprise from about 1% by weight to about 95% by weight of a food product. In some embodiments, proteins may comprise from about 1% by weight to about 95% by weight of a food product. In some embodiments, a food product may comprise proteins in a range with high and low values independently selected from about 1%, 3%, 5%, 8%, 10%, 15%, 20%, 25%, 30%, 35%, 40%, 45%, 50%, 55%, 60%, 65%, 70%, 75%, 80%, 85%, 90%, 95% or ranges including, between, and/or spanning the aforementioned values.

In some embodiments, the food product may include a starch. In some embodiments, food products may additionally comprise a starch ingredient(s) in amounts sufficient to provide about 5% to 45%, or about 10%-30%, or about 15%-25 starch in the food products. Starchy components may include not only pure added cereal flours or other granulations but also any starchy fraction provided by other ingredients such as oat bran or soy protein. In some embodiments, a starch may comprise any conventionally employed starch or cereal flour ingredient, for use in a ready-to-eat cereal. Exemplary suitable starchy cereals include cereal flours from major cereal grains including wheat, rice, corn (maize), oats, barley, rye, or starch fractions isolated from the cereal flowers including, for example cornstarch, wheat starch, rice starch, and various treated starches including pre-gelatinized starches and/or modified starches.

In some embodiments, the food product may include a flour. Several types of flour may be used according to the disclosure. For example, common types of flour used in food products, include all-purpose flour (which like most other flour may include protein content), Whole wheat flour, Bread flour, Cake flour, pastry flour, self-rising flour, and gluten-free flour. According to the disclosure, the concentration of flour may vary depending on the intended use of a product. For example, in baked goods like bread, cakes, and cookies, flour is typically a main ingredient and may be included at concentrations ranging from 50% to 100% of the total dry ingredients. In soups and sauces, flour is often used as a thickener and may be included at concentrations ranging from 1% to 5% of the total ingredients. Batter and breading: In fried foods like chicken or fish, flour is often used as part of the batter or breading and may be included at concentrations ranging from 20% to 50% of the total dry ingredients. In snack foods, like crackers and chips, flour may be included at concentrations ranging from 30% to 70% of the total ingredients. In some embodiments, flour may comprise from about 1% by weight to about 95% by weight of a food product. In some embodiments, flours may comprise from about 1% by weight to about 95% by weight of a food product. In some embodiments, a food product may comprise flour in a range with high and low values independently selected from about 1%, 3%, 5%, 8%, 10%, 15%, 20%, 25%, 30%, 35%, 40%, 45%, 50%, 55%, 60%, 65%, 70%, 75%, 80%, 85%, 90%, 95% or ranges including, between, and/or spanning the aforementioned values.

In some embodiments, the food product may include a fat. In some embodiments, the food product may include an oil. In some embodiments, the food product may include one or more fats or oils. Several types of fats may be used according to the disclosure. For example, common types of fats include butter, margarine, vegetable oils, shortening, and lard. According to the disclosure, the concentration of fats may vary depending on the intended use of a product. In baked goods like cakes, cookies, and pastries, fats like butter, shortening, or oil are often used to provide moisture, flavor, and texture. In some embodiments, Fats may be included at concentrations ranging from 10% to 30% of the total ingredients. In some embodiments, like in sauces and dressings, fats like olive oil or mayonnaise are often used to provide flavor and texture. In some embodiments, Fats may be included at concentrations ranging from 10% to 30% of the total ingredients. In fried foods like chicken or French fries, fats like vegetable oil or lard are used for frying and may be included at concentrations ranging from 30% to 60% of the total ingredients. In snack foods like chips and crackers, fats like vegetable oil or palm oil are often used to provide flavor and texture. In some embodiments, Fats may be included at concentrations ranging from 10% to 30% of the total ingredients. In some embodiments, fats may comprise from about 1% by weight to about 95% by weight of a food product. In some embodiments, fats may comprise from about 1% by weight to about 95% by weight of a food product. In some embodiments, a food product may comprise fat in a range with high and low values independently selected from about 1%, 3%, 5%, 8%, 10%, 15%, 20%, 25%, 30%, 35%, 40%, 45%, 50%, 55%, 60%, 65%, 70%, 75%, 80%, 85%, 90%, 95% or ranges including, between, and/or spanning the aforementioned values.

In some embodiments, the food product may include one or more additional ingredients. If desired, the disclosed food products may additionally include a variety of materials designed to improve their aesthetic or nutritional qualities. These adjuvant materials can include vitamin and/or mineral fortification, colors, flavors, sweetener(s), and mixtures thereof. The precise ingredient concentration may vary. Generally, however, such materials can each include about 0.01% to about 5%, or about 0.1% to 2% dry weight of a food product. One especially useful material is common salt. In some embodiments, salt comprises about 0.1 to 5%, or about 0.5 to 4.0% of the food products.

In some embodiments, the food product is derived from a plant. In some embodiments, the food product is derived from hemp hulls. In some embodiments, the food product is a solid food. In some embodiments, the food product is a semi-solid food. In some embodiments, the food product is a puffed product, a bakery product, a pressed cake, a cooked product, a food bar, a cereal, a crisp, or a spread.

A dietary supplement is a product taken by mouth that contains a compound or extract of the disclosure and is intended to supplement the diet. A nutraceutical is a product derived from a food source that provides extra health benefits, in addition to the basic nutritional value found in the food. A pharmaceutical composition is defined as any component of a drug product intended to furnish pharmacological activity or other direct effect in the diagnosis, cure, mitigation, treatment, or prevention of disease, or to affect the structure or any function of the body of humans or other animals. Dietary supplements, nutraceuticals and pharmaceutical compositions can be found in many capsules, forms such as tablets, coated tablets, pills, capsules, pellets, granules, softgels, gelcaps, liquids, powders, emulsions, suspensions, elixirs, syrup, and any other form suitable for use.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, a compound described herein, including a compound of Formula (I), (II), or a pharmaceutically acceptable salt thereof, can be administered orally. In some embodiments, a compound described herein, including a compound of Formula (I), (II), or a pharmaceutically acceptable salt thereof, can be provided in a form for oral consumption.

One may also provide or administer the one or more compounds, salt and/or composition in a local rather than systemic manner, for example, via injection or implantation of the compound directly into the affected area, often in a depot or sustained release formulation. Furthermore, one may provide or administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical excipient may also be prepared, placed in an appropriate container, and labeled for treatment of an indicated condition.

The compounds, salt and/or pharmaceutical composition can be provided to an administering physician or other health care professional in the form of a kit. The kit is a package which houses a container which contains the compound(s) in a suitable pharmaceutical composition, and instructions for administering the pharmaceutical composition to a subject. The kit can optionally also contain one or more additional therapeutic agents. The kit can also contain separate doses of a compound(s) or pharmaceutical composition for serial or sequential administration. The kit can optionally contain one or more diagnostic tools and instructions for use. The kit can contain suitable delivery devices, for example, syringes, and the like, along with instructions for administering the compound(s) and any other therapeutic agent. The kit can optionally contain instructions for storage, reconstitution (if applicable), and administration of any or all therapeutic agents included. The kits can include a plurality of containers reflecting the number of administrations to be given to a subject.

In some embodiments, a compound of Formula (I) or Formula (II) is administered at a dose in the range of about 0.1-200 mg/kg body weight. In some embodiments, a compound of Formula (I) or Formula (II) is provided or administered at a dose in the range of about 0.1-1, 0.5-1, 0.1-10, 0.5-10, 1-10, 1-20, 1-30, 1-40, 1-50, 1-60, 1-70, 1-80, 1-90, 1-100, 1-200, 1-300, 1-400, 1-500, 1-600, 1-700, 1-800, 1-900, 1-1000, 1-11, 1-12, 1-13, 1-13, 1-14, 1-15, 1-16, 1-17, 1-18, 1-19, 10-20, 10-30, 10-40, 10-50, 10-60, 10-70, 10-80, 10-90, 10-100, 10-200, 10-300, 10-400, 10-500, 10-600, 10-700, 10-800, 10-900, 10-1000, 20-30, 20-40, 20-50, 20-60, 20-70, 20-80, 20-90, 20-100, 20-200, 20-300, 20-400, 20-500, 20-600, 20-700, 20-800, 20-900, 20-1000, 30-40, 30-50, 30-60, 30-70, 30-80, 30-90, 30-100, 30-200, 30-300, 30-400, 30-500, 30-600, 30-700, 30-800, 30-900, 30-1000, 40-50, 40-60, 40-70, 40-80, 40-90, 40-100, 40-200, 40-300, 40-400, 40-500, 40-600, 40-700, 40-800, 40-900, 40-1000, 50-60, 50-70, 50-80, 50-90, 50-100, 50-200, 50-300, 50-400, 50-500, 50-600, 50-700, 50-800, 50-900, 60-70, 60-80, 60-90, 60-100, 60-200, 60-300, 60-400, 60-500, 60-600, 60-700, 60-800, 60-900, 60-1000, 70-80, 70-90, 70-100, 70-200, 70-300, 70-400, 70-500, 70-600, 70-700, 70-800, 70-900, 70-1000, 80-90, 80-100, 80-200, 80-300, 80-400, 80-500, 80-600, 80-700, 80-800, 80-900, 80-100, 90-100, 90-200, 90-300, 90-400, 90-500, 90-600, 90-700, 90-800, 90-900, 90-1000, 100-150, 100-200, 100-300, 100-400, 100-500, 100-600, 100-700, 100-800, 100-900, or 100-1000 mg/kg of body weight. In some embodiments, a compound of Formula (I) or Formula (II) is provided or administered at a dose of about 0.01, 0.02, 0.03, 0.05, 0.07, 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50, 55, 60, 65, 70, 80, 90, or 95 mg/kg of the body weight. In some embodiments, a compound of Formula (I) or Formula (II) is provided or administered at a dose less than about 0.01, 0.02, 0.03, 0.05, 0.07, 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, a compound of Formula (I) or Formula (II) is provided or administered at a dose greater than about 0.01, 0.02, 0.03, 0.05, 0.07, 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mg/kg of a subjects body weight.

In some embodiments, a compound of Formula (I) or Formula (II) dose is about 0.1 mg-10 mg, 0.1 mg-25 mg, 0.1 mg-30 mg, 0.1 mg-50 mg, 0.1 mg-75 mg, 0.1 mg-100 mg, 0.5 mg-10 mg, 0.5 mg-25 mg, 0.5 mg-30 mg, 0.5 mg-50 mg, 0.5 mg-75 mg, 0.5 mg-100 mg, 1 mg-10 mg, 1 mg-25 mg, 1 mg-30 mg, 1 mg-50 mg, 1 mg-75 mg, 1 mg-100 mg, 2 mg-10 mg, 2 mg-25 mg, 2 mg-30 mg, 2 mg-50 mg, 2 mg-75 mg, 2 mg-100 mg, 3 mg-10 mg, 3 mg-25 mg, 3 mg-30 mg, 3 mg-50 mg, 3 mg-75 mg, 3 mg-100 mg, 4 mg-100 mg, 5 mg-10 mg, 5 mg-25 mg, 5 mg-30 mg, 5 mg-50 mg, 5 mg-75 mg, 5 mg-300 mg, 5 mg-200 mg, 7.5 mg-15 mg, 7.5 mg-25 mg, 7.5 mg-30 mg, 7.5 mg-50 mg, 7.5 mg-75 mg, 7.5 mg-100 mg, 7.5 mg-200 mg, 10 mg-20 mg, 10 mg-25 mg, 10 mg-50 mg, 10 mg-75 mg, 10 mg-100 mg, 15 mg-30 mg, 15 mg-50 mg, 15 mg-100 mg, 20 mg-20 mg, 20 mg-100 mg, 30 mg-100 mg, 40 mg-100 mg, 10 mg-80 mg, 15 mg-80 mg, 20 mg-80 mg, 30 mg-80 mg, 40 mg-80 mg, 10 mg-60 mg, 15 mg-60 mg, 20 mg-60 mg, 30 mg-60 mg, or about 40 mg-60 mg. In some embodiments, a compound of Formula (I) or Formula (II) provided or administered is about 20 mg-60 mg, 27 mg-60 mg, 20 mg-45 mg, or 27 mg-45 mg. In some embodiments, a compound of Formula (I) or Formula (II) provided or administered is about 1 mg-5 mg, 1 mg-7.5 mg, 2.5 mg-5 mg, 2.5 mg-7.5 mg, 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, a compound of Formula (I) or Formula (II) dose is greater than, equal to, or about 0.1 mg, 0.3 mg, 0.5 mg, 0.75 mg, 1 mg, 1.25 mg, 1.5 mg, 1.75 mg, 2 mg, 2.5 mg, 3 mg, 3.5 mg, 4 mg, 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, about 200 mg, about 300 mg. about 400 mg, about 500 mg, about 600 mg, about 700 mg, about 800 mg, about 900 mg, or about 1000 mg. In some embodiments, a compound of Formula (I) or Formula (II) dose is about less than about 0.5 mg, 0.75 mg, 1 mg, 1.25 mg, 1.5 mg, 1.75 mg, 2 mg, 2.5 mg, 3 mg, 3.5 mg, 4 mg, 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg.

In some embodiments, the formulation includes an excipient. In some embodiments, the excipient is a nutraceutically acceptable excipient. In some embodiments, the nutraceutically acceptable excipient is selected from medium chain triglycerides, diglycerides, and monoglycerides, caprylic acid, linoleic acid, linoleic acid, oleic acid, keto-oleic acid. In some embodiments, the nutraceutically acceptable excipient further includes a second fatty acid component having one or more non-activated fatty acids selected from linoleic acid, α-linoleic acid, γ-linoleic acid, oleic acid, eicosapentaenoic acid (EPA), docosahexaenoic acid (DEIA), or derivatives thereof.

In some embodiments, the formulation includes a carrier. In some embodiments, the carrier is a nutraceutically acceptable carrier. Each carrier should be compatible with the other ingredients of the formulation and not injurious to the subject. Some examples of materials that can serve as carriers include: (1) sugars, such as lactose, glucose and sucrose; (2) starches, such as corn starch and potato starch; (3) cellulose, and its derivatives, such as sodium carboxymethyl cellulose, ethyl cellulose, cellulose acetate, and hydroxyl propyl methyl cellulose; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other nontoxic compatible substances employed in conventional formulations. In some embodiments, the carrier is about 0.5% to about 80% (w/w) of the formulation. In some embodiments, the carrier is about 0.5%, about 1.0%, about 1.5%, about 2.0%, about 2.5%, about 3.0%, about 3.5%, about 4.0%, about 4.5%, about 5.0%, about 7.5%, about 10%, about 15%, about 20%, about 25%, about 30%, about 35%, about 40%, about 45%, about 50%, about 55%, about 60%, about 70%, about 75%, about 80% (w/w) of the formulation, or ranges including, between, and/or spanning the aforementioned values.

For preparing solid compositions such as tablets or capsules, the compound or extract is mixed with a carrier (e.g., conventional tableting ingredients such as corn starch, lactose, sucrose, sorbitol, talc, stearic acid, magnesium stearate, dicalcium phosphate or gums) and other diluents (e.g., water) to form a solid composition. This solid composition is then subdivided into unit dosage forms containing an effective amount of the compound of the present disclosure. The tablets or pills containing the compound or extract can be coated or otherwise compounded to provide a dosage form affording the advantage of prolonged action.

In particular embodiments of this disclosure, a consumable composition includes the one or more compounds or an extract comprising one or more compounds described herein, a carrier and a preservative to reduce or retard microbial growth. In some embodiments, the preservative is about 0.01% to about 5% (w/w) of the formulation. In some embodiments, the preservative is about 0.5%, about 1.0%, about 1.5%, about 2.0%, about 2.5%, about 3.0%, about 3.5%, about 4.0%, about 4.5%, about 5.0% w/w of the formulation, or ranges including, between, and/or spanning the aforementioned values. In some embodiments, the preservative is added in amounts up to about 5%, for example, from about 0.01% to 1% (w/w). In some embodiments, the preservative may include sodium benzoate, methyl parabens, propyl parabens, sodium nitrite, sulphur dioxide, sodium sorbate and potassium sorbate. Other suitable preservatives include, but are not limited to, salts of edetate, (also known as salts of ethylenediaminetetraacetic acid, or EDTA, such a disodium EDTA), or a combination thereof.

In some embodiments, the formulation is a liquid. In some embodiments, the formulation is a liquid product. In some embodiments, the liquid product is produced from hemp hulls. In some embodiments, the liquid product is produced from a recombinant cell. In some embodiments, the liquid product is a beverage. In some embodiments, the formulation may be incorporated into a milk-based beverage. In some embodiments, the formulation may be incorporated into a sports drink beverage. In some embodiments, the formulation may be incorporated into a fruit juice beverage. In some embodiments, the formulation may be incorporated into an alcoholic beverage. In some embodiments, the formulation may be incorporated into a water-based beverage. In some embodiments, the formulation may be a smoothie. In some embodiments, the liquid may include one or more compounds or an extract of the disclosure. In some embodiments, the liquid may be incorporated for oral administration or consumption. In some embodiments, the liquid may include aqueous solution, suitably flavored syrups, aqueous or oil suspensions, and flavored emulsions with edible oils as well as elixirs and similar vehicles. Suitable dispersing or suspending agents for aqueous suspensions include synthetic natural gums, such as tragacanth, acacia, alginate, dextran, sodium carboxymethyl cellulose, methylcellulose, polyvinylpyrrolidone or gelatin. Liquid preparations for oral administration or consumption may take the form of, for example, solutions, syrups or suspensions, or they may be presented as a dry product for reconstitution with water or other suitable vehicles before use. Such liquid preparations may be prepared by conventional means with acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

Methods of preparing formulations or compositions of this disclosure include the step of bringing into association a compound or extract of the present disclosure with the carrier and, optionally, one or more accessory and/or active ingredients. In general, the formulations are prepared by uniformly and intimately bringing into association a compound or extract of the present disclosure with liquid carriers, or finely divided solid carriers, or both, and then, if necessary, shaping the product. As such, the disclosed formulation may consist of, or consist essentially of a compound or extract described herein in combination with a suitable carrier.

In some embodiments, the formulation includes one or more compounds or an extract of the present disclosure at about 0.1, 0.5, 1, 2, 3, 4, 5, 6, 7, 8, 9, 10, 15, 20, 25, 30, 35, 40, 45, 50, 55, 60, 65, 70, 75, 80, 85, 90, 91, 92, 93, 94, 95, 96, 97, 98, 99% w/w, or ranges including and/or spanning the aforementioned values. In some embodiments, the formulation includes from about 0.1% to about 10% of one or more compounds or an extract as described herein. In some embodiments, the formulation includes from about 0.1% to about 5% of one or more compounds or an extract as described herein. In some embodiments, the formulation includes from about 1% to about 10% of one or more compounds or an extract as described herein. In some embodiments, when one or more compounds or an extract of the present disclosure is provided or administered as pharmaceuticals, nutraceuticals, or dietary supplements to humans and animals, they can be given per se or as a composition containing, for example, 0.1 to 99% active ingredient in combination with an acceptable carrier. In some embodiments, the compound or extract of the present disclosure may be provided or administered at about 0.1, 0.5, 1, 2, 3, 4, 5, 6, 7, 8, 9, 10, 15, 20, 25, 30, 35, 40, 45, 50, 55, 60, 65, 70, 75, 80, 85, 90, 91, 92, 93, 94, 95, 96, 97, 98, 99% w/w, or ranges including and/or spanning the aforementioned values.

A consumable product may be consumed by a subject to provide less than 100 mg of one or more compounds disclosed herein per day. In certain embodiments, the consumable provides between about 1 mg/day and about 60 mg/day of a tyramine containing hydroxycinnamic acid amide. In certain embodiments, the consumable provides between about 10 and about 60 mg/day of a tyramine containing hydroxycinnamic acid amide. The effective amount can be established by methods known in the art and be dependent upon bioavailability, toxicity, etc.

While it is contemplated that individual tyramine containing hydroxycinnamic acid amides may be used in the consumables of this disclosure, it is further contemplated that two or more of the compounds or extracts could be combined in any relative amounts to produce custom combinations of ingredients containing two or more tyramine containing hydroxycinnamic acid amides in desired ratios to enhance product efficacy, improve organoleptic properties or some other measure of quality important to the ultimate use of the product.

In some embodiments, the formulation may include one or more compounds as described herein and a suitable carrier or excipient. In some embodiments, the one or more compounds described herein may be in a ratio from about 10:1 to about 1:10, or ranges including and/or spanning the aforementioned values. In some embodiments, the one or more compounds described herein may be in a ratio from about 10:1, 9.5:1, 8.5:1, 8.0:1, 7.5:1, 7:1, 6.5:1, 6:1, 5.5:1, 5:1, 4.5:1, 4:1, 3.5:1, 3.0:1, 2.5:1, 2.0:1, 1.5:1, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, 1:10. In some embodiments, the one or more compounds may be selected from N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, and 5-hydroxyferuloyltyramine, a salt, or isomer thereof. In some embodiments, the one or more compounds is N-trans-caffeoyltyramine. In some embodiments, the one or more compounds is N-trans-feruloyltyramine. In some embodiments, the one or more compounds includes N-trans-caffeoyltyramine and N-trans-feruloyltyramine. In some embodiments, the ratio of N-trans-caffeoyltyramine to N-trans-feruloyltyramine is from about 10:1 to about 1:10. In some embodiments, the ratio of N-trans-caffeoyltyramine to N-trans-feruloyltyramine is from about 5:1 to about 1:5. In some embodiments, the N-trans-caffeoyltyramine and the N-trans-feruloyltyramine is in a ratio from about 2.5:1 to about 2:1. In some embodiments, the ratio of N-trans-caffeoyltyramine to N-trans-feruloyltyramine is from about 2.5:1 to about 1:2.5. In some embodiments, the ratio of N-trans-caffeoyltyramine to N-trans-feruloyltyramine is about 5:1. In some embodiments, the ratio of N-trans-caffeoyltyramine to N-trans-feruloyltyramine is from about 4:1. In some embodiments, the ratio of N-trans-caffeoyltyramine to N-trans-feruloyltyramine is about 3:1. In some embodiments, the ratio of N-trans-caffeoyltyramine to N-trans-feruloyltyramine is about 2.2:1. In some embodiments, the ratio of N-trans-caffeoyltyramine to N-trans-feruloyltyramine is about 2:1. In some embodiments, the ratio of N-trans-caffeoyltyramine to N-trans-feruloyltyramine is about 1:2. In some embodiments, the ratio of N-trans-caffeoyltyramine to N-trans-feruloyltyramine is about 1:2.2. In some embodiments, the ratio of N-trans-caffeoyltyramine to N-trans-feruloyltyramine is about 1:3. In some embodiments, the ratio of N-trans-caffeoyltyramine to N-trans-feruloyltyramine is about 1:4. In some embodiments, the ratio of N-trans-caffeoyltyramine to N-trans-feruloyltyramine is about 1:5.

### Methods

In aspects, the disclosure also provides methods for improving, restoring, or maintaining a subject's gut health. In accordance with such methods, an effective amount of one or more compounds, a formulation, or an extract including one or more compounds of this disclosure is provided to a subject in need thereof so that the subject's digestive function is improved or maintained thereby addressing the underlying pathogenesis of one or more gut health disorders and promoting the health, well-being, and quality of life of the subject. Accordingly, provided herein are compounds, compositions, formulations, and methods for treating, maintaining, improving, or restoring conditions including, but not limited to, metabolic syndrome, reducing intestinal inflammation, increasing epithelial turnover, reducing microbial dysbiosis, tight junction modulation, anti-microbial peptide secretion, goblet cell mucus production, intestinal barrier function, intestinal barrier integrity, gut permeability, gut barrier function, and targeted reduction of enteropathogens.

In embodiments, a method for treating, improving, restoring, preventing, or reducing metabolic syndrome or a metabolic disorder is provided. In some embodiments, the method for treating, improving, restoring, preventing, or reducing a metabolic disorder in a subject in need thereof comprises providing or administering one or more compounds, a composition, a formulation, or an extract comprising one or more compounds as described herein. In some embodiments, the one or more compounds, composition, formulation, or extract is administered in amounts that together are sufficient to treat, prevent, or reduce metabolic syndrome or a metabolic disorder. In some embodiments, the one or more compounds may be selected from N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, and 5-hydroxyferuloyltyramine, a salt, or isomer thereof. In some embodiments, the one or more compounds is N-trans-caffeoyltyramine. In some embodiments, the one or more compounds is N-trans-feruloyltyramine. In some embodiments, the one or more compounds includes N-trans-caffeoyltyramine and N-trans-feruloyltyramine. In some embodiments, the ratio of N-trans-caffeoyltyramine to N-trans-feruloyltyramine is from about 10:1 to about 1:10. In some embodiments, the ratio of N-trans-caffeoyltyramine to N-trans-feruloyltyramine is from about 5:1 to about 1:5. In some embodiments, the N-trans-caffeoyltyramine and the N-trans-feruloyltyramine is in a ratio from about 2.5:1 to about 2:1. In some embodiments, the ratio of N-trans-caffeoyltyramine to N-trans-feruloyltyramine is from about 2.5:1 to about 1:2.5. In some embodiments, the ratio of N-trans-caffeoyltyramine to N-trans-feruloyltyramine is about 5:1. In some embodiments, the ratio of N-trans-caffeoyltyramine to N-trans-feruloyltyramine is about 4:1. In some embodiments, the ratio of N-trans-caffeoyltyramine to N-trans-feruloyltyramine is about 3:1. In some embodiments, the ratio of N-trans-caffeoyltyramine to N-trans-feruloyltyramine is about 2.2:1. In some embodiments, the ratio of N-trans-caffeoyltyramine to N-trans-feruloyltyramine is about 2:1. In some embodiments, the ratio of N-trans-caffeoyltyramine to N-trans-feruloyltyramine is about 1:2. In some embodiments, the ratio of N-trans-caffeoyltyramine to N-trans-feruloyltyramine is about 1:2.2. In some embodiments, the ratio of N-trans-caffeoyltyramine to N-trans-feruloyltyramine is about 1:3. In some embodiments, the ratio of N-trans-caffeoyltyramine to N-trans-feruloyltyramine is about 1:4. In some embodiments, the ratio of N-trans-caffeoyltyramine to N-trans-feruloyltyramine is about 1:5. In some embodiments, the ratio of N-trans-caffeoyltyramine to N-trans-feruloyltyramine is about 1:5. In some embodiments, the composition is formulated as a dietary supplement, food ingredient or additive, a food product, a medical food, nutraceutical or pharmaceutical composition. In some embodiments, the composition is provided in a unit dosage form and is configured for administration between 0.1 and 100 mg/kg of the body weight of the subject of Formula (I) per administration. In some embodiments, the food product is a food bar. Use of a composition for treating, improving, restoring, preventing, or reducing metabolic syndrome or a metabolic disorder, wherein the composition includes N-trans-caffeoyltyramine. Use of a composition for treating, improving, restoring, preventing, or reducing metabolic syndrome or a metabolic disorder, wherein the composition includes N-trans-feruloyltyramine. Use of a composition for treating, improving, restoring, preventing, or reducing metabolic syndrome or a metabolic disorder, wherein the composition includes N-trans-caffeoyltyramine and N-trans-feruloyltyramine. Use of a composition for treating, improving, restoring, preventing, or reducing metabolic syndrome or a metabolic disorder, wherein the composition includes N-trans-caffeoyltyramine and N-trans-feruloyltyramine in a ratio from about 10:1 to about 1:10, or from about 5:1 to about 1:5, or from about 5:1 to about 1:1, or from about 2.5:1 to about 1:1, or from about 2:5:1 to about 2:1, or about 10:1, about 9:1, about 8:1, about 7:1, about 6:1, about 5:1, about 4:1, about 3:1, about 2.5:1, about 2.2:1, about 2:1, about 1:1, about 1:2, about 1:3, about 1:4, about 1:5, about 1:6, about 1:7, about 1:8, about 1:9, or about 1:10.

In embodiments, a method for treating, improving, preventing, or reducing intestinal inflammation is provided. Intestinal inflammation is a response to harmful stimuli throughout the intestine. It is usually marked by increase in interleukin 6 (IL-6) leading to swelling of the tissue. This swelling has been linked to many intestinal disorders such as inflammatory bowel disease, ulcer formation, and irritable bowel syndrome, to name a few. Additionally, inflammation inhibits nutrient absorption by impairing gut barrier transmission. In some embodiments, intestinal inflammation is gut inflammation. In some embodiments, the method for treating, improving, preventing, or reducing an intestinal inflammation in a subject in need thereof comprises providing or administering one or more compounds, a composition, a formulation, or an extract including or more compounds as described herein. In some embodiments, the one or more compounds, composition, formulation, or extract is provided or administered in amounts that together are sufficient to treat, improve, prevent, or reduce intestinal inflammation. In some embodiments, the one or more compounds may be selected from N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, and 5-hydroxyferuloyltyramine, a salt, or isomer thereof. In some embodiments, the one or more compounds is N-trans-caffeoyltyramine. In some embodiments, the one or more compounds is N-trans-feruloyltyramine. In some embodiments, the one or more compounds includes N-trans-caffeoyltyramine and N-trans-feruloyltyramine. In some embodiments, the ratio of N-trans-caffeoyltyramine to N-trans-feruloyltyramine is from about 10:1 to about 1:10. In some embodiments, the ratio of N-trans-caffeoyltyramine to N-trans-feruloyltyramine is from about 5:1 to about 1:5. In some embodiments, the N-trans-caffeoyltyramine and the N-trans-feruloyltyramine is in a ratio from about 2.5:1 to about 2:1. In some embodiments, the ratio of N-trans-caffeoyltyramine to N-trans-feruloyltyramine is from about 2.5:1 to about 1:2.5. In some embodiments, the ratio of N-trans-caffeoyltyramine to N-trans-feruloyltyramine is about 5:1. In some embodiments, the ratio of N-trans-caffeoyltyramine to N-trans-feruloyltyramine is about 4:1. In some embodiments, the ratio of N-trans-caffeoyltyramine to N-trans-feruloyltyramine is about 3:1. In some embodiments, the ratio of N-trans-caffeoyltyramine to N-trans-feruloyltyramine is about 2.2:1. In some embodiments, the ratio of N-trans-caffeoyltyramine to N-trans-feruloyltyramine is about 2:1. In some embodiments, the ratio of N-trans-caffeoyltyramine to N-trans-feruloyltyramine is about 1:2. In some embodiments, the ratio of N-trans-caffeoyltyramine to N-trans-feruloyltyramine is about 1:2.2. In some embodiments, the ratio of N-trans-caffeoyltyramine to N-trans-feruloyltyramine is about 1:3. In some embodiments, the ratio of N-trans-caffeoyltyramine to N-trans-feruloyltyramine is about 1:4. In some embodiments, the ratio of N-trans-caffeoyltyramine to N-trans-feruloyltyramine is about 1:5. In some embodiments, the ratio of N-trans-caffeoyltyramine to N-trans-feruloyltyramine is about 1:5. In some embodiments, the composition is formulated as a dietary supplement, food ingredient or additive, a food product, a medical food, nutraceutical or pharmaceutical composition. In some embodiments, the composition is provided in a unit dosage form and is configured for administration between 0.1 and 100 mg/kg of the body weight of the subject of Formula (I) per administration. In some embodiments, the food product is a food bar. Use of a composition for treating, improving, restoring, preventing, or reducing intestinal inflammation, wherein the composition includes N-trans-caffeoyltyramine. Use of a composition for treating, improving, restoring, preventing, or reducing intestinal inflammation, wherein the composition includes N-trans-feruloyltyramine. Use of a composition for treating, improving, restoring, preventing, or reducing intestinal inflammation, wherein the composition includes N-trans-caffeoyltyramine and N-trans-feruloyltyramine. Use of a composition for treating, improving, restoring, preventing, or reducing intestinal inflammation, wherein the composition includes N-trans-caffeoyltyramine and N-trans-feruloyltyramine in a ratio from about 10:1 to about 1:10, or from about 5:1 to about 1:5, or from about 5:1 to about 1:1, or from about 2.5:1 to about 1:1, or from about 2:5:1 to about 2:1, or about 10:1, about 9:1, about 8:1, about 7:1, about 6:1, about 5:1, about 4:1, about 3:1, about 2.5:1, about 2.2:1, about 2:1, about 1:1, about 1:2, about 1:3, about 1:4, about 1:5, about 1:6, about 1:7, about 1:8, about 1:9, or about 1:10.

In embodiments, a method for treating, improving, restoring, preventing, or increasing epithelial turnover is provided. Epithelial turnover is the process of cell loss to balance new cell generation. It is crucial in epithelial health and avoiding mutations that can lead to many negative health indications. In some embodiments, the method for treating, improving, restoring, preventing, or increasing epithelial turnover in a subject in need thereof comprises administering one or more compounds, a composition, a formulation, or an extract including one or more compounds as described herein. In some embodiments, the composition is provided or administered in amounts that together are sufficient to treat, improve, restore, prevent, or increase epithelial turnover. In some embodiments, the one or more compounds may be selected from N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, and 5-hydroxyferuloyltyramine, a salt, or isomer thereof. In some embodiments, the one or more compounds is N-trans-caffeoyltyramine. In some embodiments, the one or more compounds is N-trans-feruloyltyramine. In some embodiments, the one or more compounds includes N-trans-caffeoyltyramine and N-trans-feruloyltyramine. In some embodiments, the ratio of N-trans-caffeoyltyramine to N-trans-feruloyltyramine is from about 10:1 to about 1:10. In some embodiments, the ratio of N-trans-caffeoyltyramine to N-trans-feruloyltyramine is from about 5:1 to about 1:5. In some embodiments, the N-trans-caffeoyltyramine and the N-trans-feruloyltyramine is in a ratio from about 2.5:1 to about 2:1. In some embodiments, the ratio of N-trans-caffeoyltyramine to N-trans-feruloyltyramine is from about 2.5:1 to about 1:2.5. In some embodiments, the ratio of N-trans-caffeoyltyramine to N-trans-feruloyltyramine is about 5:1. In some embodiments, the ratio of N-trans-caffeoyltyramine to N-trans-feruloyltyramine is about 4:1. In some embodiments, the ratio of N-trans-caffeoyltyramine to N-trans-feruloyltyramine is about 3:1. In some embodiments, the ratio of N-trans-caffeoyltyramine to N-trans-feruloyltyramine is about 2.2:1. In some embodiments, the ratio of N-trans-caffeoyltyramine to N-trans-feruloyltyramine is about 2:1. In some embodiments, the ratio of N-trans-caffeoyltyramine to N-trans-feruloyltyramine is about 1:2. In some embodiments, the ratio of N-trans-caffeoyltyramine to N-trans-feruloyltyramine is about 1:2.2. In some embodiments, the ratio of N-trans-caffeoyltyramine to N-trans-feruloyltyramine is about 1:3. In some embodiments, the ratio of N-trans-caffeoyltyramine to N-trans-feruloyltyramine is about 1:4. In some embodiments, the ratio of N-trans-caffeoyltyramine to N-trans-feruloyltyramine is about 1:5. In some embodiments, the ratio of N-trans-caffeoyltyramine to N-trans-feruloyltyramine is about 1:5. In some embodiments, the composition is formulated as a dietary supplement, food ingredient or additive, a food product, a medical food, nutraceutical or pharmaceutical composition. In some embodiments, the composition is provided in a unit dosage form and is configured for administration between 0.1 and 100 mg/kg of the body weight of the subject of Formula (I) per administration. In some embodiments, the food product is a food bar. Use of a composition for treating, improving, restoring, preventing, or reducing epithelial turnover, wherein the composition includes N-trans-caffeoyltyramine. Use of a composition for treating, improving, restoring, preventing, or reducing epithelial turnover, wherein the composition includes N-trans-feruloyltyramine. Use of a composition for treating, improving, restoring, preventing, or reducing epithelial turnover, wherein the composition includes N-trans-caffeoyltyramine and N-trans-feruloyltyramine. Use of a composition for treating, improving, restoring, preventing, or reducing epithelial turnover, wherein the composition includes N-trans-caffeoyltyramine and N-trans-feruloyltyramine in a ratio from about 10:1 to about 1:10, or from about 5:1 to about 1:5, or from about 5:1 to about 1:1, or from about 2.5:1 to about 1:1, or from about 2:5:1 to about 2:1, or about 10:1, about 9:1, about 8:1, about 7:1, about 6:1, about 5:1, about 4:1, about 3:1, about 2.5:1, about 2.2:1, about 2:1, about 1:1, about 1:2, about 1:3, about 1:4, about 1:5, about 1:6, about 1:7, about 1:8, about 1:9, or about 1:10.

In embodiments, a method for treating, improving, restoring, preventing, or reducing microbial dysbiosis is provided. The gut microbiome is the collection of bacteria and other microbes that reside in the gastrointestinal tract. A healthy microbiome has been found to drastically improve overall health by breaking down unabsorbable compounds into healthful products that can easily pass through the intestinal epithelium. Due to this, a healthy microbiome is essential for proper nutrient absorption. In some embodiments, the method for treating, improving, restoring, preventing, or reducing microbial dysbiosis in a subject in need thereof comprises providing or administering one or more compounds, a composition, a formulation, or an extract including one or more compounds as described herein. In some embodiments, the one or more compounds, composition, formulation, or extract is provided or administered in amounts that together are sufficient to treat, prevent, or reduce microbial dysbiosis. In some embodiments, the gastrointestinal epithelial cell barrier function disorder is at least one selected from the group consisting of: inflammatory bowel disease, Crohn's disease, ulcerative colitis, pouchitis, irritable bowel syndrome, enteric infections, Clostridium difficile infections, metabolic diseases, obesity, type 2 diabetes, non-alcoholic steatohepatitis, non-alcoholic fatty liver disease, liver disorders, alcoholic steatohepatitis, celiac disease, necrotizing enterocolitis, gastro intestinal disorders, short bowel syndrome, GI mucositis, chemotherapy induced mucositis, radiation induced mucositis, oral mucositis, interstitial cystitis, neurological disorders, cognitive disorders, Alzheimer's, Parkinson's, multiple sclerosis, autism, chemotherapy associated steatohepatitis (CASH), and pediatric versions of the aforementioned diseases. In some embodiments, the subject experiences a reduction in at least one symptom associated with the gastrointestinal epithelial cell barrier function disorder selected from the group consisting of: abdominal pain, blood in stool, pus in stool, fever, weight loss, frequent diarrhea, fatigue, reduced appetite, tenesmus, and rectal bleeding. In some embodiments, the one or more compounds may be selected from N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, and 5-hydroxyferuloyltyramine, a salt, or isomer thereof. In some embodiments, the one or more compounds is N-trans-caffeoyltyramine. In some embodiments, the one or more compounds is N-trans-feruloyltyramine. In some embodiments, the one or more compounds includes N-trans-caffeoyltyramine and N-trans-feruloyltyramine. In some embodiments, the ratio of N-trans-caffeoyltyramine to N-trans-feruloyltyramine is from about 10:1 to about 1:10. In some embodiments, the ratio of N-trans-caffeoyltyramine to N-trans-feruloyltyramine is from about 5:1 to about 1:5. In some embodiments, the N-trans-caffeoyltyramine and the N-trans-feruloyltyramine is in a ratio from about 2.5:1 to about 2:1. In some embodiments, the ratio of N-trans-caffeoyltyramine to N-trans-feruloyltyramine is from about 2.5:1 to about 1:2.5. In some embodiments, the ratio of N-trans-caffeoyltyramine to N-trans-feruloyltyramine is about 5:1. In some embodiments, the ratio of N-trans-caffeoyltyramine to N-trans-feruloyltyramine is about 4:1. In some embodiments, the ratio of N-trans-caffeoyltyramine to N-trans-feruloyltyramine is about 3:1. In some embodiments, the ratio of N-trans-caffeoyltyramine to N-trans-feruloyltyramine is about 2.2:1. In some embodiments, the ratio of N-trans-caffeoyltyramine to N-trans-feruloyltyramine is about 2:1. In some embodiments, the ratio of N-trans-caffeoyltyramine to N-trans-feruloyltyramine is about 1:2. In some embodiments, the ratio of N-trans-caffeoyltyramine to N-trans-feruloyltyramine is about 1:2.2. In some embodiments, the ratio of N-trans-caffeoyltyramine to N-trans-feruloyltyramine is about 1:3. In some embodiments, the ratio of N-trans-caffeoyltyramine to N-trans-feruloyltyramine is about 1:4. In some embodiments, the ratio of N-trans-caffeoyltyramine to N-trans-feruloyltyramine is about 1:5. In some embodiments, the ratio of N-trans-caffeoyltyramine to N-trans-feruloyltyramine is about 1:5. In some embodiments, the composition is formulated as a dietary supplement, food ingredient or additive, a food product, a medical food, nutraceutical or pharmaceutical composition. In some embodiments, the composition is provided in a unit dosage form and is configured for administration between 0.1 and 100 mg/kg of the body weight of the subject of Formula (I) per administration. In some embodiments, the food product is a food bar. Use of a composition for treating, improving, restoring, preventing, or reducing microbial dysbiosis, wherein the composition includes N-trans-caffeoyltyramine. Use of a composition for treating, improving, restoring, preventing, or reducing microbial dysbiosis, wherein the composition includes N-trans-feruloyltyramine. Use of a composition for treating, improving, restoring, preventing, or reducing microbial dysbiosis, wherein the composition includes N-trans-caffeoyltyramine and N-trans-feruloyltyramine. Use of a composition for treating, improving, restoring, preventing, or reducing microbial dysbiosis, wherein the composition includes N-trans-caffeoyltyramine and N-trans-feruloyltyramine in a ratio from about 10:1 to about 1:10, or from about 5:1 to about 1:5, or from about 5:1 to about 1:1, or from about 2.5:1 to about 1:1, or from about 2:5:1 to about 2:1, or about 10:1, about 9:1, about 8:1, about 7:1, about 6:1, about 5:1, about 4:1, about 3:1, about 2.5:1, about 2.2:1, about 2:1, about 1:1, about 1:2, about 1:3, about 1:4, about 1:5, about 1:6, about 1:7, about 1:8, about 1:9, or about 1:10.

In embodiments, a method for treating, improving, restoring, preventing, or improving tight junction modulation is provided. The intestinal epithelial cells are bound together by tight junction proteins that alter the tightness of the cell layer. When these junctions are functioning properly, they allow for the passage of nutrients essential to maintain human health while blocking harmful compounds from entering circulation. Improper function of tight junction proteins can result in loose junctions and a leaky gut, or overly-tight junctions that impede nutrient absorption. In some embodiments, the method for treating, preventing, or improving tight junction modulation in a subject in need thereof comprises providing or administering one or more compounds, a composition, a formulation, or an extract including one or more compounds as described herein as described herein. In some embodiments, the one or more compounds, composition, formulation, or extract is provided or administered in amounts that together are sufficient to treat, improve, restore, prevent, or improve tight junction modulation. In some embodiments, the one or more compounds may be selected from N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, and 5-hydroxyferuloyltyramine, a salt, or isomer thereof. In some embodiments, the one or more compounds is N-trans-caffeoyltyramine. In some embodiments, the one or more compounds is N-trans-feruloyltyramine. In some embodiments, the one or more compounds includes N-trans-caffeoyltyramine and N-trans-feruloyltyramine. In some embodiments, the ratio of N-trans-caffeoyltyramine to N-trans-feruloyltyramine is from about 10:1 to about 1:10. In some embodiments, the ratio of N-trans-caffeoyltyramine to N-trans-feruloyltyramine is from about 5:1 to about 1:5. In some embodiments, the N-trans-caffeoyltyramine and the N-trans-feruloyltyramine is in a ratio from about 2.5:1 to about 2:1. In some embodiments, the ratio of N-trans-caffeoyltyramine to N-trans-feruloyltyramine is from about 2.5:1 to about 1:2.5. In some embodiments, the ratio of N-trans-caffeoyltyramine to N-trans-feruloyltyramine is about 5:1. In some embodiments, the ratio of N-trans-caffeoyltyramine to N-trans-feruloyltyramine is about 4:1. In some embodiments, the ratio of N-trans-caffeoyltyramine to N-trans-feruloyltyramine is about 3:1. In some embodiments, the ratio of N-trans-caffeoyltyramine to N-trans-feruloyltyramine is about 2.2:1. In some embodiments, the ratio of N-trans-caffeoyltyramine to N-trans-feruloyltyramine is about 2:1. In some embodiments, the ratio of N-trans-caffeoyltyramine to N-trans-feruloyltyramine is about 1:2. In some embodiments, the ratio of N-trans-caffeoyltyramine to N-trans-feruloyltyramine is about 1:2.2. In some embodiments, the ratio of N-trans-caffeoyltyramine to N-trans-feruloyltyramine is about 1:3. In some embodiments, the ratio of N-trans-caffeoyltyramine to N-trans-feruloyltyramine is about 1:4. In some embodiments, the ratio of N-trans-caffeoyltyramine to N-trans-feruloyltyramine is about 1:5. In some embodiments, the ratio of N-trans-caffeoyltyramine to N-trans-feruloyltyramine is about 1:5. In some embodiments, the ratio of N-trans-caffeoyltyramine to N-trans-feruloyltyramine is from about 1:5. In some embodiments, the composition is formulated as a dietary supplement, food ingredient or additive, a food product, a medical food, nutraceutical or pharmaceutical composition. In some embodiments, the composition is provided in a unit dosage form and is configured for administration between 0.1 and 100 mg/kg of the body weight of the subject of Formula (I) per administration. In some embodiments, the food product is a food bar. Use of a composition for treating, improving, restoring, preventing, or reducing tight junction modulation, wherein the composition includes N-trans-caffeoyltyramine. Use of a composition for treating, improving, restoring, preventing, or reducing tight junction modulation, wherein the composition includes N-trans-feruloyltyramine. Use of a composition for treating, improving, restoring, preventing, or reducing tight junction modulation, wherein the composition includes N-trans-caffeoyltyramine and N-trans-feruloyltyramine. Use of a composition for treating, improving, restoring, preventing, or reducing tight junction modulation, wherein the composition includes N-trans-caffeoyltyramine and N-trans-feruloyltyramine in a ratio from about 10:1 to about 1:10, or from about 5:1 to about 1:5, or from about 5:1 to about 1:1, or from about 2.5:1 to about 1:1, or from about 2:5:1 to about 2:1, or about 10:1, about 9:1, about 8:1, about 7:1, about 6:1, about 5:1, about 4:1, about 3:1, about 2.5:1, about 2.2:1, about 2:1, about 1:1, about 1:2, about 1:3, about 1:4, about 1:5, about 1:6, about 1:7, about 1:8, about 1:9, or about 1:10.

In embodiments, a method for treating, improving, preventing, or reducing anti-microbial peptide secretion is provided. To defend the human body from harmful compounds in the gut, anti-microbial peptides are secreted throughout the GI tract. These peptides are essential to reducing pathogens in the body that may lead to infection. In some embodiments, the method for treating, improving, preventing, or reducing anti-microbial peptide secretion in a subject in need thereof comprises providing or administering one or more compounds, a composition, a formulation, or an extract including one or more compounds as described herein. In some embodiments, the composition is provided or administered in amounts that together are sufficient to treat, improve, prevent, or reduce anti-microbial peptide secretion. In some embodiments, the one or more compounds may be selected from N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, and 5-hydroxyferuloyltyramine, a salt, or isomer thereof. In some embodiments, the one or more compounds is N-trans-caffeoyltyramine. In some embodiments, the one or more compounds is N-trans-feruloyltyramine. In some embodiments, the ratio of N-trans-caffeoyltyramine to N-trans-feruloyltyramine is from about 10:1 to about 1:10. In some embodiments, the ratio of N-trans-caffeoyltyramine to N-trans-feruloyltyramine is from about 5:1 to about 1:5. In some embodiments, the N-trans-caffeoyltyramine and the N-trans-feruloyltyramine is in a ratio from about 2.5:1 to about 2:1. In some embodiments, the ratio of N-trans-caffeoyltyramine to N-trans-feruloyltyramine is from about 2.5:1 to about 1:2.5. In some embodiments, the ratio of N-trans-caffeoyltyramine to N-trans-feruloyltyramine is about 5:1. In some embodiments, the ratio of N-trans-caffeoyltyramine to N-trans-feruloyltyramine is about 4:1. In some embodiments, the ratio of N-trans-caffeoyltyramine to N-trans-feruloyltyramine is about 3:1. In some embodiments, the ratio of N-trans-caffeoyltyramine to N-trans-feruloyltyramine is about 2.2:1. In some embodiments, the ratio of N-trans-caffeoyltyramine to N-trans-feruloyltyramine is about 2:1. In some embodiments, the ratio of N-trans-caffeoyltyramine to N-trans-feruloyltyramine is about 1:2. In some embodiments, the ratio of N-trans-caffeoyltyramine to N-trans-feruloyltyramine is about 1:2.2. In some embodiments, the ratio of N-trans-caffeoyltyramine to N-trans-feruloyltyramine is about 1:3. In some embodiments, the ratio of N-trans-caffeoyltyramine to N-trans-feruloyltyramine is about 1:4. In some embodiments, the ratio of N-trans-caffeoyltyramine to N-trans-feruloyltyramine is about 1:5. In some embodiments, the ratio of N-trans-caffeoyltyramine to N-trans-feruloyltyramine is about 1:5. In some embodiments, the composition is formulated as a dietary supplement, food ingredient or additive, a food product, a medical food, nutraceutical or pharmaceutical composition. In some embodiments, the composition is provided in a unit dosage form and is configured for administration between 0.1 and 100 mg/kg of the body weight of the subject of Formula (I) per administration. In some embodiments, the food product is a food bar.

In aspects, a method for treating, improving, restoring, preventing, or increasing goblet cell mucus production is provided. The epithelial lining is protected by a layer of mucus, which is the first line of defense for any irritants that may damage the gut barrier. Goblet cells are essential in generating and maintaining this mucus layer, thus are essential in protecting the epithelium from harm. In some embodiments, the method for treating, improving, restoring, preventing, or increasing goblet cell mucus production in a subject in need thereof comprises providing or administering one or more compounds, a composition, a formulation, or an extract including one or more compounds as described herein. In some embodiments, the composition is provided or administered in amounts that together are sufficient to treat, improve, restore, prevent, or increase goblet cell mucus production. In some embodiments, the one or more compounds may be selected from N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, and 5-hydroxyferuloyltyramine, a salt, or isomer thereof. In some embodiments, the one or more compounds is N-trans-caffeoyltyramine. In some embodiments, the one or more compounds is N-trans-feruloyltyramine. In some embodiments, the ratio of N-trans-caffeoyltyramine to N-trans-feruloyltyramine is from about 10:1 to about 1:10. In some embodiments, the ratio of N-trans-caffeoyltyramine to N-trans-feruloyltyramine is from about 5:1 to about 1:5. In some embodiments, the N-trans-caffeoyltyramine and the N-trans-feruloyltyramine is in a ratio from about 2.5:1 to about 2:1. In some embodiments, the ratio of N-trans-caffeoyltyramine to N-trans-feruloyltyramine is from about 2.5:1 to about 1:2.5. In some embodiments, the ratio of N-trans-caffeoyltyramine to N-trans-feruloyltyramine is about 5:1. In some embodiments, the ratio of N-trans-caffeoyltyramine to N-trans-feruloyltyramine is about 4:1. In some embodiments, the ratio of N-trans-caffeoyltyramine to N-trans-feruloyltyramine is about 3:1. In some embodiments, the ratio of N-trans-caffeoyltyramine to N-trans-feruloyltyramine is about 2.2:1. In some embodiments, the ratio of N-trans-caffeoyltyramine to N-trans-feruloyltyramine is about 2:1. In some embodiments, the ratio of N-trans-caffeoyltyramine to N-trans-feruloyltyramine is about 1:2. In some embodiments, the ratio of N-trans-caffeoyltyramine to N-trans-feruloyltyramine is about 1:2.2. In some embodiments, the ratio of N-trans-caffeoyltyramine to N-trans-feruloyltyramine is about 1:3. In some embodiments, the ratio of N-trans-caffeoyltyramine to N-trans-feruloyltyramine is about 1:4. In some embodiments, the ratio of N-trans-caffeoyltyramine to N-trans-feruloyltyramine is about 1:5. In some embodiments, the ratio of N-trans-caffeoyltyramine to N-trans-feruloyltyramine is about 1:5. In some embodiments, the composition is formulated as a dietary supplement, food ingredient or additive, a food product, a medical food, nutraceutical or pharmaceutical composition. In some embodiments, the composition is provided in a unit dosage form and is configured for administration between 0.1 and 100 mg/kg of the body weight of the subject of Formula (I) per administration. In some embodiments, the food product is a food bar. Use of a composition for treating, improving, restoring, preventing, or increasing goblet cell mucus production, wherein the composition includes N-trans-caffeoyltyramine. Use of a composition for treating, improving, restoring, preventing, or increasing goblet cell mucus production, wherein the composition includes N-trans-feruloyltyramine. Use of a composition for treating, improving, restoring, preventing, or increasing goblet cell mucus production, wherein the composition includes N-trans-caffeoyltyramine and N-trans-feruloyltyramine. Use of a composition for treating, improving, restoring, preventing, or increasing goblet cell mucus production, wherein the composition includes N-trans-caffeoyltyramine and N-trans-feruloyltyramine in a ratio from about 10:1 to about 1:10, or from about 5:1 to about 1:5, or from about 5:1 to about 1:1, or from about 2.5:1 to about 1:1, or from about 2:5:1 to about 2:1, or about 10:1, about 9:1, about 8:1, about 7:1, about 6:1, about 5:1, about 4:1, about 3:1, about 2.5:1, about 2.2:1, about 2:1, about 1:1, about 1:2, about 1:3, about 1:4, about 1:5, about 1:6, about 1:7, about 1:8, about 1:9, or about 1:10.

In aspects, a method for treating, improving, restoring, preventing, or targeted reduction of enteropathogens is provided. Similar to anti-microbial secretion and microbiome health, enteropathogen reduction is a process by which the GI tract protects itself from damage by harmful compounds. Enteropathogens are known to cause inflammation, damage the epithelium and lead to infection throughout the body. Reducing the population of enteropathogens throughout the GI tract is essential to maintaining whole body health and proper nutrient uptake. In some embodiments, the method for treating, improving, restoring, preventing, or targeted reduction of enteropathogens in a subject in need thereof comprises providing or administering one or more compounds, a composition, a formulation, or an extract including one or more compounds as described herein. In some embodiments, the one or more compounds, composition, formulation, or extract is administered in amounts that together are sufficient to treat, improve, restore, prevent, or targeted reduction of enteropathogens. In some embodiments, the one or more compounds may be selected from N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, and 5-hydroxyferuloyltyramine, a salt, or isomer thereof. In some embodiments, the one or more compounds is N-trans-caffeoyltyramine. In some embodiments, the one or more compounds is N-trans-feruloyltyramine. In some embodiments, the one or more compounds includes N-trans-caffeoyltyramine and N-trans-feruloyltyramine. In some embodiments, the ratio of N-trans-caffeoyltyramine to N-trans-feruloyltyramine is from about 10:1 to about 1:10. In some embodiments, the ratio of N-trans-caffeoyltyramine to N-trans-feruloyltyramine is from about 5:1 to about 1:5. In some embodiments, the N-trans-caffeoyltyramine and the N-trans-feruloyltyramine is in a ratio from about 2.5:1 to about 2:1. In some embodiments, the ratio of N-trans-caffeoyltyramine to N-trans-feruloyltyramine is from about 2.5:1 to about 1:2.5. In some embodiments, the ratio of N-trans-caffeoyltyramine to N-trans-feruloyltyramine is about 5:1. In some embodiments, the ratio of N-trans-caffeoyltyramine to N-trans-feruloyltyramine is about 4:1. In some embodiments, the ratio of N-trans-caffeoyltyramine to N-trans-feruloyltyramine is about 3:1. In some embodiments, the ratio of N-trans-caffeoyltyramine to N-trans-feruloyltyramine is about 2.2:1. In some embodiments, the ratio of N-trans-caffeoyltyramine to N-trans-feruloyltyramine is about 2:1. In some embodiments, the ratio of N-trans-caffeoyltyramine to N-trans-feruloyltyramine is about 1:2. In some embodiments, the ratio of N-trans-caffeoyltyramine to N-trans-feruloyltyramine is about 1:2.2. In some embodiments, the ratio of N-trans-caffeoyltyramine to N-trans-feruloyltyramine is about 1:3. In some embodiments, the ratio of N-trans-caffeoyltyramine to N-trans-feruloyltyramine is about 1:4. In some embodiments, the ratio of N-trans-caffeoyltyramine to N-trans-feruloyltyramine is about 1:5. In some embodiments, the composition is formulated as a dietary supplement, food ingredient or additive, a food product, a medical food, nutraceutical or pharmaceutical composition. In some embodiments, the composition is provided in a unit dosage form and is configured for administration between 0.1 and 100 mg/kg of the body weight of the subject of Formula (I) per administration. In some embodiments, the food product is a food bar. Use of a composition for treating, improving, restoring, preventing, or reducing enteropathogens, wherein the composition includes N-trans-caffeoyltyramine. Use of a composition for treating, improving, restoring, preventing, or reducing enteropathogens, wherein the composition includes N-trans-feruloyltyramine. Use of a composition for treating, improving, restoring, preventing, or increasing goblet cell mucus production, wherein the composition includes N-trans-caffeoyltyramine and N-trans-feruloyltyramine. Use of a composition for treating, improving, restoring, preventing, or reducing enteropathogens, wherein the composition includes N-trans-caffeoyltyramine and N-trans-feruloyltyramine in a ratio from about 10:1 to about 1:10, or from about 5:1 to about 1:5, or from about 5:1 to about 1:1, or from about 2.5:1 to about 1:1, or from about 2:5:1 to about 2:1, or about 10:1, about 9:1, about 8:1, about 7:1, about 6:1, about 5:1, about 4:1, about 3:1, about 2.5:1, about 2.2:1, about 2:1, about 1:1, about 1:2, about 1:3, about 1:4, about 1:5, about 1:6, about 1:7, about 1:8, about 1:9, or about 1:10.

In some aspects, providing or administering one or more compounds or a formulation as described herein promotes a strong gut barrier. In some embodiments, the one or more compounds may be selected from N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, and 5-hydroxyferuloyltyramine, a salt, or isomer thereof. In some embodiments, the one or more compounds is N-trans-caffeoyltyramine. In some embodiments, the one or more compounds is N-trans-feruloyltyramine. In some embodiments, the one or more compounds includes N-trans-caffeoyltyramine and N-trans-feruloyltyramine. In some embodiments, the ratio of N-trans-caffeoyltyramine to N-trans-feruloyltyramine is from about 10:1 to about 1:10. In some embodiments, the ratio of N-trans-caffeoyltyramine to N-trans-feruloyltyramine is from about 5:1 to about 1:5. In some embodiments, the N-trans-caffeoyltyramine and the N-trans-feruloyltyramine is in a ratio from about 2.5:1 to about 2:1. In some embodiments, the ratio of N-trans-caffeoyltyramine to N-trans-feruloyltyramine is from about 2.5:1 to about 1:2.5. In some embodiments, the ratio of N-trans-caffeoyltyramine to N-trans-feruloyltyramine is about 5:1. In some embodiments, the ratio of N-trans-caffeoyltyramine to N-trans-feruloyltyramine is about 4:1. In some embodiments, the ratio of N-trans-caffeoyltyramine to N-trans-feruloyltyramine is about 3:1. In some embodiments, the ratio of N-trans-caffeoyltyramine to N-trans-feruloyltyramine is about 2.2:1. In some embodiments, the ratio of N-trans-caffeoyltyramine to N-trans-feruloyltyramine is about 2:1. In some embodiments, the ratio of N-trans-caffeoyltyramine to N-trans-feruloyltyramine is about 1:2. In some embodiments, the ratio of N-trans-caffeoyltyramine to N-trans-feruloyltyramine is about 1:2.2. In some embodiments, the ratio of N-trans-caffeoyltyramine to N-trans-feruloyltyramine is about 1:3. In some embodiments, the ratio of N-trans-caffeoyltyramine to N-trans-feruloyltyramine is about 1:4. In some embodiments, the ratio of N-trans-caffeoyltyramine to N-trans-feruloyltyramine is about 1:5. In some embodiments, the composition is formulated as a dietary supplement, food ingredient or additive, a food product, a medical food, nutraceutical or pharmaceutical composition. In some embodiments, the composition is provided in a unit dosage form and is configured for administration between 0.1 and 100 mg/kg of the body weight of the subject of Formula (I) per administration. In some embodiments, the food product is a food bar. Use of a composition for promoting a strong gut barrier, wherein the composition includes N-trans-caffeoyltyramine. Use of a composition for promoting a strong gut barrier, wherein the composition includes N-trans-feruloyltyramine. Use of a composition for promoting a strong gut barrier, wherein the composition includes N-trans-caffeoyltyramine and N-trans-feruloyltyramine. Use of a composition for promoting a strong gut barrier, wherein the composition includes N-trans-caffeoyltyramine and N-trans-feruloyltyramine in a ratio from about 10:1 to about 1:10, or from about 5:1 to about 1:5, or from about 5:1 to about 1:1, or from about 2.5:1 to about 1:1, or from about 2:5:1 to about 2:1, or about 10:1, about 9:1, about 8:1, about 7:1, about 6:1, about 5:1, about 4:1, about 3:1, about 2.5:1, about 2.2:1, about 2:1, about 1:1, about 1:2, about 1:3, about 1:4, about 1:5, about 1:6, about 1:7, about 1:8, about 1:9, or about 1:10.

In some aspects, providing or administering one or more compounds or a formulation as described herein regulates the transport of essential molecules and restrict passage of harmful substances. In some embodiments, the one or more compounds may be selected from N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, and 5-hydroxyferuloyltyramine, a salt, or isomer thereof. In some embodiments, the one or more compounds is N-trans-caffeoyltyramine. In some embodiments, the one or more compounds is N-trans-feruloyltyramine. In some embodiments, the one or more compounds includes N-trans-caffeoyltyramine and N-trans-feruloyltyramine. In some embodiments, the ratio of N-trans-caffeoyltyramine to N-trans-feruloyltyramine is from about 10:1 to about 1:10. In some embodiments, the ratio of N-trans-caffeoyltyramine to N-trans-feruloyltyramine is from about 5:1 to about 1:5. In some embodiments, the N-trans-caffeoyltyramine and the N-trans-feruloyltyramine is in a ratio from about 2.5:1 to about 2:1. In some embodiments, the ratio of N-trans-caffeoyltyramine to N-trans-feruloyltyramine is from about 2.5:1 to about 1:2.5. In some embodiments, the ratio of N-trans-caffeoyltyramine to N-trans-feruloyltyramine is about 5:1. In some embodiments, the ratio of N-trans-caffeoyltyramine to N-trans-feruloyltyramine is about 4:1. In some embodiments, the ratio of N-trans-caffeoyltyramine to N-trans-feruloyltyramine is about 3:1. In some embodiments, the ratio of N-trans-caffeoyltyramine to N-trans-feruloyltyramine is about 2.2:1. In some embodiments, the ratio of N-trans-caffeoyltyramine to N-trans-feruloyltyramine is about 2:1. In some embodiments, the ratio of N-trans-caffeoyltyramine to N-trans-feruloyltyramine is about 1:2. In some embodiments, the ratio of N-trans-caffeoyltyramine to N-trans-feruloyltyramine is about 1:2.2. In some embodiments, the ratio of N-trans-caffeoyltyramine to N-trans-feruloyltyramine is about 1:3. In some embodiments, the ratio of N-trans-caffeoyltyramine to N-trans-feruloyltyramine is about 1:4. In some embodiments, the ratio of N-trans-caffeoyltyramine to N-trans-feruloyltyramine is about 1:5. In some embodiments, the composition is formulated as a dietary supplement, food ingredient or additive, a food product, a medical food, nutraceutical or pharmaceutical composition. In some embodiments, the composition is provided in a unit dosage form and is configured for administration between 0.1 and 100 mg/kg of the body weight of the subject of Formula (I) per administration. In some embodiments, the food product is a food bar. Use of a composition for treating, improving, restoring, preventing, or regulating the transport of essential molecules and restrict passage of harmful substances, wherein the composition includes N-trans-caffeoyltyramine. Use of a composition for treating, improving, restoring, preventing, or regulating the transport of essential molecules and restrict passage of harmful substances, wherein the composition includes N-trans-feruloyltyramine. Use of a composition for treating, improving, restoring, preventing, or regulating the transport of essential molecules and restrict passage of harmful substances, wherein the composition includes N-trans-caffeoyltyramine and N-trans-feruloyltyramine. Use of a composition for treating, improving, restoring, preventing, or regulating the transport of essential molecules and restrict passage of harmful substances, wherein the composition includes N-trans-caffeoyltyramine and N-trans-feruloyltyramine in a ratio from about 10:1 to about 1:10, or from about 5:1 to about 1:5, or from about 5:1 to about 1:1, or from about 2.5:1 to about 1:1, or from about 2:5:1 to about 2:1, or about 10:1, about 9:1, about 8:1, about 7:1, about 6:1, about 5:1, about 4:1, about 3:1, about 2.5:1, about 2.2:1, about 2:1, about 1:1, about 1:2, about 1:3, about 1:4, about 1:5, about 1:6, about 1:7, about 1:8, about 1:9, or about 1:10.

In some aspects, providing or administering one or more compounds or a formulation as described herein maintains intestinal barrier function. In some embodiments, the one or more compounds may be selected from N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, and 5-hydroxyferuloyltyramine, a salt, or isomer thereof. In some embodiments, the one or more compounds is N-trans-caffeoyltyramine. In some embodiments, the one or more compounds is N-trans-feruloyltyramine. In some embodiments, the ratio of N-trans-caffeoyltyramine to N-trans-feruloyltyramine is from about 10:1 to about 1:10. In some embodiments, the ratio of N-trans-caffeoyltyramine to N-trans-feruloyltyramine is from about 5:1 to about 1:5. In some embodiments, the N-trans-caffeoyltyramine and the N-trans-feruloyltyramine is in a ratio from about 2.5:1 to about 2:1. In some embodiments, the ratio of N-trans-caffeoyltyramine to N-trans-feruloyltyramine is from about 2.5:1 to about 1:2.5. In some embodiments, the ratio of N-trans-caffeoyltyramine to N-trans-feruloyltyramine is about 5:1. In some embodiments, the ratio of N-trans-caffeoyltyramine to N-trans-feruloyltyramine is about 4:1. In some embodiments, the ratio of N-trans-caffeoyltyramine to N-trans-feruloyltyramine is about 3:1. In some embodiments, the ratio of N-trans-caffeoyltyramine to N-trans-feruloyltyramine is about 2.2:1. In some embodiments, the ratio of N-trans-caffeoyltyramine to N-trans-feruloyltyramine is about 2:1. In some embodiments, the ratio of N-trans-caffeoyltyramine to N-trans-feruloyltyramine is about 1:2. In some embodiments, the ratio of N-trans-caffeoyltyramine to N-trans-feruloyltyramine is about 1:2.2. In some embodiments, the ratio of N-trans-caffeoyltyramine to N-trans-feruloyltyramine is about 1:3. In some embodiments, the ratio of N-trans-caffeoyltyramine to N-trans-feruloyltyramine is about 1:4. In some embodiments, the ratio of N-trans-caffeoyltyramine to N-trans-feruloyltyramine is about 1:5. In some embodiments, the composition is formulated as a dietary supplement, food ingredient or additive, a food product, a medical food, nutraceutical or pharmaceutical composition. In some embodiments, the composition is provided in a unit dosage form and is configured for administration between 0.1 and 100 mg/kg of the body weight of the subject of Formula (I) per administration. In some embodiments, the food product is a food bar. Use of a composition for treating, improving, restoring, preventing, or maintaining intestinal barrier function, wherein the composition includes N-trans-caffeoyltyramine. Use of a composition for treating, improving, restoring, preventing, or maintaining intestinal barrier function, wherein the composition includes N-trans-feruloyltyramine. Use of a composition for treating, improving, restoring, preventing, or maintaining intestinal barrier function, wherein the composition includes N-trans-caffeoyltyramine and N-trans-feruloyltyramine. Use of a composition for treating, improving, restoring, preventing, or maintaining intestinal barrier function s, wherein the composition includes N-trans-caffeoyltyramine and N-trans-feruloyltyramine in a ratio from about 10:1 to about 1:10, or from about 5:1 to about 1:5, or from about 5:1 to about 1:1, or from about 2.5:1 to about 1:1, or from about 2:5:1 to about 2:1, or about 10:1, about 9:1, about 8:1, about 7:1, about 6:1, about 5:1, about 4:1, about 3:1, about 2.5:1, about 2.2:1, about 2:1, about 1:1, about 1:2, about 1:3, about 1:4, about 1:5, about 1:6, about 1:7, about 1:8, about 1:9, or about 1:10.

In some aspects, providing or administering one or more compounds or a formulation as described herein regulates intestinal barrier integrity. In some embodiments, the one or more compounds may be selected from N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, and 5-hydroxyferuloyltyramine, a salt, or isomer thereof. In some embodiments, the one or more compounds is N-trans-caffeoyltyramine. In some embodiments, the one or more compounds is N-trans-feruloyltyramine. In some embodiments, the one or more compounds includes N-trans-caffeoyltyramine and N-trans-feruloyltyramine. In some embodiments, the ratio of N-trans-caffeoyltyramine to N-trans-feruloyltyramine is from about 10:1 to about 1:10. In some embodiments, the ratio of N-trans-caffeoyltyramine to N-trans-feruloyltyramine is from about 5:1 to about 1:5. In some embodiments, the N-trans-caffeoyltyramine and the N-trans-feruloyltyramine is in a ratio from about 2.5:1 to about 2:1. In some embodiments, the ratio of N-trans-caffeoyltyramine to N-trans-feruloyltyramine is from about 2.5:1 to about 1:2.5. In some embodiments, the ratio of N-trans-caffeoyltyramine to N-trans-feruloyltyramine is about 5:1. In some embodiments, the ratio of N-trans-caffeoyltyramine to N-trans-feruloyltyramine is about 4:1. In some embodiments, the ratio of N-trans-caffeoyltyramine to N-trans-feruloyltyramine is about 3:1. In some embodiments, the ratio of N-trans-caffeoyltyramine to N-trans-feruloyltyramine is about 2.2:1. In some embodiments, the ratio of N-trans-caffeoyltyramine to N-trans-feruloyltyramine is about 2:1. In some embodiments, the ratio of N-trans-caffeoyltyramine to N-trans-feruloyltyramine is about 1:2. In some embodiments, the ratio of N-trans-caffeoyltyramine to N-trans-feruloyltyramine is about 1:2.2. In some embodiments, the ratio of N-trans-caffeoyltyramine to N-trans-feruloyltyramine is about 1:3. In some embodiments, the ratio of N-trans-caffeoyltyramine to N-trans-feruloyltyramine is about 1:4. In some embodiments, the ratio of N-trans-caffeoyltyramine to N-trans-feruloyltyramine is about 1:5. In some embodiments, the composition is formulated as a dietary supplement, food ingredient or additive, a food product, a medical food, nutraceutical or pharmaceutical composition. In some embodiments, the composition is provided in a unit dosage form and is configured for administration between 0.1 and 100 mg/kg of the body weight of the subject of Formula (I) per administration. In some embodiments, the food product is a food bar. Use of a composition for treating, improving, restoring, preventing, or regulating intestinal barrier integrity, wherein the composition includes N-trans-caffeoyltyramine. Use of a composition for treating, improving, restoring, preventing, or regulating intestinal barrier integrity, wherein the composition includes N-trans-feruloyltyramine. Use of a composition for treating, improving, restoring, preventing, or regulating intestinal barrier integrity, wherein the composition includes N-trans-caffeoyltyramine and N-trans-feruloyltyramine. Use of a composition for treating, improving, restoring, preventing, or regulating intestinal barrier integrity, wherein the composition includes N-trans-caffeoyltyramine and N-trans-feruloyltyramine in a ratio from about 10:1 to about 1:10, or from about 5:1 to about 1:5, or from about 5:1 to about 1:1, or from about 2.5:1 to about 1:1, or from about 2:5:1 to about 2:1, or about 10:1, about 9:1, about 8:1, about 7:1, about 6:1, about 5:1, about 4:1, about 3:1, about 2.5:1, about 2.2:1, about 2:1, about 1:1, about 1:2, about 1:3, about 1:4, about 1:5, about 1:6, about 1:7, about 1:8, about 1:9, or about 1:10.

In some aspects, providing or administering one or more compounds or a formulation as described herein improves gut permeability. In some embodiments, the one or more compounds may be selected from N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, and 5-hydroxyferuloyltyramine, a salt, or isomer thereof. In some embodiments, the one or more compounds is N-trans-caffeoyltyramine. In some embodiments, the one or more compounds is N-trans-feruloyltyramine. In some embodiments, the one or more compounds includes N-trans-caffeoyltyramine and N-trans-feruloyltyramine. In some embodiments, the ratio of N-trans-caffeoyltyramine to N-trans-feruloyltyramine is from about 10:1 to about 1:10. In some embodiments, the ratio of N-trans-caffeoyltyramine to N-trans-feruloyltyramine is from about 5:1 to about 1:5. In some embodiments, the N-trans-caffeoyltyramine and the N-trans-feruloyltyramine is in a ratio from about 2.5:1 to about 2:1. In some embodiments, the ratio of N-trans-caffeoyltyramine to N-trans-feruloyltyramine is from about 2.5:1 to about 1:2.5. In some embodiments, the ratio of N-trans-caffeoyltyramine to N-trans-feruloyltyramine is about 5:1. In some embodiments, the ratio of N-trans-caffeoyltyramine to N-trans-feruloyltyramine is about 4:1. In some embodiments, the ratio of N-trans-caffeoyltyramine to N-trans-feruloyltyramine is about 3:1. In some embodiments, the ratio of N-trans-caffeoyltyramine to N-trans-feruloyltyramine is about 2.2:1. In some embodiments, the ratio of N-trans-caffeoyltyramine to N-trans-feruloyltyramine is about 2:1. In some embodiments, the ratio of N-trans-caffeoyltyramine to N-trans-feruloyltyramine is about 1:2. In some embodiments, the ratio of N-trans-caffeoyltyramine to N-trans-feruloyltyramine is about 1:2.2. In some embodiments, the ratio of N-trans-caffeoyltyramine to N-trans-feruloyltyramine is about 1:3. In some embodiments, the ratio of N-trans-caffeoyltyramine to N-trans-feruloyltyramine is about 1:4. In some embodiments, the ratio of N-trans-caffeoyltyramine to N-trans-feruloyltyramine is about 1:5. In some embodiments, the composition is formulated as a dietary supplement, food ingredient or additive, a food product, a medical food, nutraceutical or pharmaceutical composition. In some embodiments, the composition is provided in a unit dosage form and is configured for administration between 0.1 and 100 mg/kg of the body weight of the subject of Formula (I) per administration. In some embodiments, the food product is a food bar. Use of a composition for treating, improving, restoring, preventing, or regulating gut permeability, wherein the composition includes N-trans-caffeoyltyramine. Use of a composition for treating, improving, restoring, preventing, or regulating gut permeability, wherein the composition includes N-trans-feruloyltyramine. Use of a composition for treating, improving, restoring, preventing, or regulating gut permeability, wherein the composition includes N-trans-caffeoyltyramine and N-trans-feruloyltyramine. Use of a composition for treating, improving, restoring, preventing, or regulating gut permeability, wherein the composition includes N-trans-caffeoyltyramine and N-trans-feruloyltyramine in a ratio from about 10:1 to about 1:10, or from about 5:1 to about 1:5, or from about 5:1 to about 1:1, or from about 2.5:1 to about 1:1, or from about 2:5:1 to about 2:1, or about 10:1, about 9:1, about 8:1, about 7:1, about 6:1, about 5:1, about 4:1, about 3:1, about 2.5:1, about 2.2:1, about 2:1, about 1:1, about 1:2, about 1:3, about 1:4, about 1:5, about 1:6, about 1:7, about 1:8, about 1:9, or about 1:10.

In some aspects, providing or administering one or more compounds or a formulation as described herein improves gut barrier function. In some embodiments, the one or more compounds may be selected from N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, and 5-hydroxyferuloyltyramine, a salt, or isomer thereof. In some embodiments, the one or more compounds is N-trans-caffeoyltyramine. In some embodiments, the one or more compounds is N-trans-feruloyltyramine. In some embodiments, the one or more compounds includes N-trans-caffeoyltyramine and N-trans-feruloyltyramine. In some embodiments, the ratio of N-trans-caffeoyltyramine to N-trans-feruloyltyramine is from about 10:1 to about 1:10. In some embodiments, the ratio of N-trans-caffeoyltyramine to N-trans-feruloyltyramine is from about 5:1 to about 1:5. In some embodiments, the N-trans-caffeoyltyramine and the N-trans-feruloyltyramine is in a ratio from about 2.5:1 to about 2:1. In some embodiments, the ratio of N-trans-caffeoyltyramine to N-trans-feruloyltyramine is from about 2.5:1 to about 1:2.5. In some embodiments, the ratio of N-trans-caffeoyltyramine to N-trans-feruloyltyramine is about 5:1. In some embodiments, the ratio of N-trans-caffeoyltyramine to N-trans-feruloyltyramine is about 4:1. In some embodiments, the ratio of N-trans-caffeoyltyramine to N-trans-feruloyltyramine is about 3:1. In some embodiments, the ratio of N-trans-caffeoyltyramine to N-trans-feruloyltyramine is about 2.2:1. In some embodiments, the ratio of N-trans-caffeoyltyramine to N-trans-feruloyltyramine is about 2:1. In some embodiments, the ratio of N-trans-caffeoyltyramine to N-trans-feruloyltyramine is about 1:2. In some embodiments, the ratio of N-trans-caffeoyltyramine to N-trans-feruloyltyramine is about 1:2.2. In some embodiments, the ratio of N-trans-caffeoyltyramine to N-trans-feruloyltyramine is about 1:3. In some embodiments, the ratio of N-trans-caffeoyltyramine to N-trans-feruloyltyramine is about 1:4. In some embodiments, the ratio of N-trans-caffeoyltyramine to N-trans-feruloyltyramine is about 1:5. In some embodiments, the composition is formulated as a dietary supplement, food ingredient or additive, a food product, a medical food, nutraceutical or pharmaceutical composition. In some embodiments, the composition is provided in a unit dosage form and is configured for administration between 0.1 and 100 mg/kg of the body weight of the subject of Formula (I) per administration. In some embodiments, the food product is a food bar. Use of a composition for treating, improving, restoring, preventing, or regulating gut barrier function, wherein the composition includes N-trans-caffeoyltyramine. Use of a composition for treating, improving, restoring, preventing, or regulating gut barrier function, wherein the composition includes N-trans-feruloyltyramine. Use of a composition for treating, improving, restoring, preventing, or regulating gut barrier function, wherein the composition includes N-trans-caffeoyltyramine and N-trans-feruloyltyramine. Use of a composition for treating, improving, restoring, preventing, or regulating gut barrier function, wherein the composition includes N-trans-caffeoyltyramine and N-trans-feruloyltyramine in a ratio from about 10:1 to about 1:10, or from about 5:1 to about 1:5, or from about 5:1 to about 1:1, or from about 2.5:1 to about 1:1, or from about 2:5:1 to about 2:1, or about 10:1, about 9:1, about 8:1, about 7:1, about 6:1, about 5:1, about 4:1, about 3:1, about 2.5:1, about 2.2:1, about 2:1, about 1:1, about 1:2, about 1:3, about 1:4, about 1:5, about 1:6, about 1:7, about 1:8, about 1:9, or about 1:10.

This disclosure also provides for improving transepithelial electrical resistance (TEER) by providing one or more compounds, a formulation, or a consumable composition as described herein and at least one carrier. In accordance with such methods, an effective amount of an extract comprising a composition as described herein is provided to a subject in need thereof thereby improving TEER in a subject. The term “subject” as used herein refers to an animal, preferably a mammal. In some embodiments, the subject is a veterinary, companion, farm, laboratory or zoological animal. In other embodiments, the subject is a human.

In some aspects, administering a composition comprising a compound of Formula (I) or Formula (II), or a pharmaceutically acceptable salt thereof, isomer, homodimer, heterodimer, or conjugate, improves TEER and decreases intestinal permeability in a subject. In some embodiments, a composition comprising a compound of Formula (I) or Formula (II) treats or ameliorates a disease or condition associated with TEER or intestinal permeability in a subject. In some embodiments, a composition comprising a compound of Formula (I) or Formula (II) treats or ameliorates a disease or condition associated with increased intestinal permeability in a subject.

In an embodiment, administering a composition comprising a compound of Formula (I) or Formula (II), or a pharmaceutically acceptable salt thereof, treats or improves at least one factor associated with TEER or intestinal permeability of a subject. In other aspects, a composition comprising a compound of Formula (I) or Formula (II), or a pharmaceutically acceptable salt thereof disclosed herein treats, ameliorates, or improves a condition or disease associated with TEER or intestinal permeability of a subject by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or ranges including and/or spanning the aforementioned values. In yet other aspects, a composition comprising Formula (I) or Formula (II), or a pharmaceutically acceptable salt thereof, treats, ameliorates, or improves a condition or disease associated with TEER or intestinal permeability of a subject by a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

The dosage may vary broadly, depending upon the desired effects and the therapeutic indication, such as marker values. Alternatively, dosages may be based and calculated upon the surface area or weight of the patient, as understood by those of skill in the art. The exact dosage will be determined on a case-by-case basis, or, in some cases, will be left to the informed discretion of the subject. The daily dosage regimen for an adult human patient may be, for example, an oral dose of a compound or composition as described herein, from about 0.01 mg to about 10000 mg, from about 1 mg to about 5000 mg, from about 5 mg to about 2000 mg, from about 10 mg to about 1000 mg, or from about 50 mg to about 500 mg. A single dose may include a compound or composition as described herein, in about 0.01 mg, about 0.1 mg, about 1 mg, about 5 mg, about 10 mg, about 20 mg, about 50 mg, about 100 mg, about 200 mg, about 300 mg, about 400 mg, about 500 mg, about 600 mg, about 800 mg, about 900 mg, about 1000 mg, about 2000 mg, about 5000 mg, or more. The dosage may be adjusted according to the body mass of the subject, for example, the dosage may be about 0.001 mg/kg, about 0.01 mg/kg, about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, or higher.

The dosage may be a single one or a series of two or more given in the course of one or more days, as is appropriate for the individual subject. In some embodiments, a compound or composition as described herein will be administered for a period of continuous therapy, for example for about a week or more (e.g., one week, two weeks, three weeks, four weeks, five weeks, six weeks, seven weeks, eight weeks, or more), for several weeks, for about a month or more (e.g., one month, two months, three months, four months, five months, six months, seven months, eight months, nine months, ten months, eleven months, twelve months, or more), for about a year or more, or for a plurality of years. In some embodiments, a compound or composition as described herein can be administered or ingested one time per day, two times per day, three times per day, or more.

As will be understood by those of skill in the art, in certain situations it may be necessary to administer the compounds disclosed herein in amounts that exceed the above-stated, preferred dosage range in order to effectively treat a subject.

Unit dosage forms can also be provided, e.g., individual packages with a premeasured amount of the composition, configured for administration on a predetermined schedule. Unit dosage forms configured for administration one to three times a day are preferred; however, in certain embodiments it may be desirable to configure the unit dosage form for administration more than three times a day, or less than one time per day.

Dosage amount and interval may be adjusted to the individual subject to provide plasma levels of the active moiety which are sufficient to maintain predetermined parameters, indicators, or marker values, or minimal effective concentration (MEC). Dosages necessary to achieve the desired result will depend on individual characteristics and route of administration. However, assays, for example, HPLC assays or bioassays, may be used to determine serum concentrations.

### Combination Therapies

In some embodiments, the compounds, compositions, or a pharmaceutical compositions disclosed herein that includes a compound described herein, or a salt or derivative thereof, may be used in combination with one or more additional active agents. Examples of additional active agents that can be used in combination with a compound or a composition as described herein that includes, but are not limited to, agents currently used for treating metabolic syndrome and related conditions, or modulating metabolism as described herein and as otherwise known to medical science. In some embodiments, a compound or a composition as described herein can be used with one, two, three or more additional active agents described herein.

In some embodiments, one or more immunomodulators or a composition as described herein can be used (for example, administered or ingested) in combination with another agent or agents for treatment, prevention, maintenance, or prophylaxis of a disease or condition described herein. For example, a compound as disclosed herein can be used in combination with one or more agents selected from albiglutide, aleglitazar, balaglitazone, canagliflozin, CJ-30001 (CJ Cheiljedang Corporation), CJ-30002 (CJ Cheiljedang Corporation), Diamyd® (glutamic acid decarboxylase (rhGAD65)), dulaglutide, exendin 4, gemigliptin, lixisenatide, lobeglitazone, shengke I (Tibet Pharmaceuticals), SK-0403 (Sanwa Kagaku Kenkyusho), teneligliptin, teplizumab, tofogliflozin, acarbose, alogliptin benzoate, chlorpropamide, Diab II (Biotech Holdings), exenatide, glibenclamide, gliclazide, glimepiride, glipizide, gliquidone, glisentide, glisolamide, HL-002 (HanAll Biopharma), insulin (human), insulin, insulin analogue (Eli Lilly®), insulin aspart, insulin detemir, insulin glargine, insulin lispro, Janumet®, linagliptin, liraglutide, metformin, miglitol, mitiglinide, nateglinide, Novo Mix 30@(Novo Nordisk®) pioglitazone, pramlintide, repaglinide, rosiglitazone maleate, saxagliptin, sitagliptin, Tresiba, tolazamide, tolbutamide, vildagliptin, voglibose, bezafibrate, diflunisal, cinnamic acid, carbutamide, glyburide (glibenclamide), glibomuride, glyhexamide, phenbutamide, and tolcyclamide or with one or more agents selected from a class of agents, where the classes include sulfonylureas, non-sulfonylurea secretagogues, glucagon-like peptides, exendin-4 polypeptides, beta 3 adrenoceptor agonists, PPAR agonists, dipeptidyl peptidase IV inhibitors, biguanides, alpha-glucosidase inhibitors, immunomodulators, statins and statin-containing combinations, angiotensin converting enzyme inhibitors, adeno sine A1 receptor agonists, adenosine A2 receptor agonists, aldosterone antagonists, alpha 1 adrenoceptor antagonists, alpha 2 adrenoceptor agonists, alpha 2 adrenoceptor agonists, angiotensin receptor antagonists, antioxidants, ATPase inhibitors, atrial peptide agonists, beta adrenoceptor antagonists, calcium channel agonists, calcium channel antagonists, diguanides, diuretics, dopamine D1 receptor agonists, endopeptidase inhibitors, endothelin receptor antagonists, guanylate cyclase stimulants, phosphodiderivativease V inhibitors, protein kinase inhibitors, Cdc2 kinase inhibitors, renin inhibitors, thromboxane synthase inhibitors, vasopeptidase inhibitors, vasopressin I antagonists, vasopressin 2 antagonists, angiogenesis inhibitors, advanced glycation end product inhibitors, bile acid binding agents, bile acid transport inhibitors, bone formation stimulants, apolipoprotein A1 agonists, DNA topoisomerase inhibitors, cholesterol absorption inhibitors, cholesterol antagonists, cholderivativeyl derivative transfer protein antagonists, cytokine synthesis inhibitors, DNA polymerase inhibitors, dopamine D2 receptor agonists, endothelin receptor antagonists, growth hormone antagonists, insulin sensitizers, lipase inhibitors, lipid peroxidation inhibitors, lipoprotein A antagonists, microsomal transport protein inhibitors, microsomal triglyceride transfer protein inhibitors, nitric oxide synthase inhibitors, oxidizing agents, phospholipase A2 inhibitors, radical formation agonists, platelet aggregation antagonists, prostaglandin synthase stimulants, reverse cholesterol transport activators, rho kinase inhibitors, selective estrogen receptor modulators, squalene epoxidase inhibitors, squalene synthase inhibitors, thromboxane A2 antagonists, amylin agonists, cannabinoid receptor antagonists, cholecystokinin A agonists, corticotropin-releasing factor agonists, dopamine uptake inhibitors, G protein-coupled receptor modulators, glutamate antagonists, glucagon-like peptide-1 agonists lipase inhibitors, melanin-concentrating hormone receptor antagonists, nerve growth factor agonists, neuropeptide Y agonists, neuropeptide Y antagonists, SNRIs, protein tyrosine phosphatase inhibitors, serotonin 2C receptor agonists, or with other agents such as central nervous system agents that affect neurotransmitters or neural ion channels, including antidepressants (bupropion), noradrenalin reuptake inhibitors (GW320659), selective serotonin 2c receptor agonists, selective 5HT 2c receptor agonists, antiseizure agents (topiramate, zonisamide), dopamine antagonists, cannabinoid-1 receptor antagonists (CB-1 receptor antagonists) (rimonabant); leptin/insulin/central nervous system pathway agents, including leptin analogues, leptin transport and/or leptin receptor promoters, ciliary neurotrophic factor (Axokine), neuropeptide Y and agouti-related peptide antagonists, pro-opiomelanocortin and cocaine and amphetamine regulated transcript promoters, α-melanocyte-stimulating hormone analogues, melanocoritin-4 receptor agonists, and agents that affect insulin metabolism/activity, which include protein-tyrosine phosphatase-IB inhibitors, peroxisome proliferator activated receptor-.gamma. receptor antagonists, short-acting bromocriptine (ergoset), somatostatin agonists (octreotide), and adiponectin/Acrp30 (Famoxin or Fatty Acid Metabolic Oxidation Inducer); gastrointestinal-neural pathway agents, including those that increase cholecystokinin activity (CCK), PYY activity, NPY activity, and PP activity, increase glucagon-like peptide-1 activity (exendin 4, dipeptidyl peptidase IV inhibitors), and those that decrease ghrelin activity, as well as amylin analogues (pramlintide); agents that may increase resting metabolic rate (selective β-3 stimulators/agonist, uncoupling protein homologues, and thyroid receptor agonists); other more diverse agents, including melanin concentrating hormone antagonists, phytostanol analogues, functional oils, P57, amylase inhibitors, growth hormone fragments, synthetic analogues of dehydroepiandrosterone sulfate, antagonists of adipocyte 11B-hydroxysteroid dehydrogenase type 1 activity, corticotropin-releasing hormone agonists, inhibitors of fatty acid synthesis (cerulenin and C75), carboxypeptidase inhibitors, indanone/indanols, aminosterols (trodusquemine/trodulamine), and other gastrointestinal lipase inhibitors (ATL962); amphetamines, such as dextroamphetamine; other sympathomimetic adrenergic agents, including phentermine, benzphetamine, phendimetrazine, mazindol, and diethylpropion; or with one or more agents selected from ecopipam; oxyntomodulin (OM); inhibitors of glucose-dependent insulinotropic polypeptide (GIP); gastrin-releasing peptide; neuromedin B; enterostatin; amfebutamone, SR-58611; CP-045598; AOD-0604; QC-BT16; rGLP-1; 1426 (HMR-1426); N-5984; ISIS-1 13715; solabegron; SR-147778; Org-34517; melanotan-II; cetilistat; c-2735; c-5093; c-2624; APD-356; radafaxine; fluasterone; GP-389255; 856464; S-2367; AVE-1625; T-71; oleoyl-estrone; peptide YY [3-36] intranasal; androgen receptor agonists; PYY 3-36; DOV-102677; tagatose; SLV-319; 1954 (Aventis Pharma AG); oxyntomodulin, Thiakis; bromocriptine, PLIVA; diabetes/hyperlipidemia therapy, Yissum; CKD-502; thyroid receptor beta agonists; beta-3 adrenoceptor agonist; CDK-A agonists; galanin antagonist; dopamine D1 D2 agonists; melanocortin modulators; verongamine; neuropeptide Y antagonists; melanin-concentrating hormone receptor antagonists; dual PPAR alpha/gamma agonists; CGEN-P-4; kinase inhibitors; human MCH receptor antagonists; GHS-R antagonists; ghrelin receptor agonists; DG70 inhibitors; cotinine; CRF-BP inhibitors; urocortin agonists; UCL-2000; impentamine; $-3 adrenergic receptor; pentapeptide MC4 agonists; trodusquemine; GT-2016; C-75; CPOP; MCH-1 receptor antagonists; RED-103004; aminosterols; orexin-1 antagonists; neuropeptide Y5 receptor antagonists; DRF-4158; PT-15; PTPase inhibitors; A37215; SA-0204; glycolipid metabolites; MC-4 agonist; produlestan; PTP-1B inhibitors; GT-2394; neuropeptide Y5 antagonists; melanocortin receptor modulators; MLN-4760; PPAR gamma/delta dual agonists; NPY5RA-972; 5-HT2C receptor agonist; neuropeptide Y5 receptor antagonists (phenyl urea analogs); AGRP/MC4 antagonists; neuropeptide Y5 antagonists (benzimidazole); glucocorticoid antagonists; MCHR1 antagonists; Acetyl-CoA carboxylase inhibitors; R-1496; HOB 1 modulators; NOX-B11; peptide YY 3-36 (eligen); 5-HT 1 modulators; pancreatic lipase inhibitors; GRC-1087; CB-1 antagonists; MCH-1 antagonists; LY-448100; bombesin BRS3 agonists; ghrelin antagonists; MC4 antagonists; stearoyl-CoA desaturase modulators; PPAR pan agonists; EP-01492; hormone-sensitive lipase inhibitors; fatty acid-binding protein 4 inhibitors; thiolactone derivatives; protein tyrosine phosphatase IB inhibitors; MCH-1 antagonist; P-64; PPAR gamma ligands; melanin concentrating hormone antagonists; thiazole gastroprokinetics; PA-452; T-226296; A-331440; immunodrug vaccines; diabetes/obesity therapeutics (Bioagency, Biofrontera Discovery GmbH); P-7 (Genfit); DT-011 M; PTP1B inhibitor; anti-diabetic peptide conjugates; KATP agonists; obesity therapeutics (Lexicon); 5-HT2 agonists; MCH-1 receptor antagonists; GMAD-1/GMAD-2; STG-a-MD; angiogenesis inhibitors; G protein-coupled receptor agonists; nicotinic therapeutics (ChemGenex); anti-obesity agents (Abbott); melanin concentrating hormone; GW-594884A; MC-4R agonist; histamine H3 antagonists; orphan GPCR modulators; MITO-3108; NLC-002; HE-2300; IGF/BBP-2-13; 5-HT2C agonists; ML-22952; neuropeptide Y receptor antagonists; AZ-40140; anti-obesity therapy (Nisshin Flour); GNTI; melanocortin receptor modulators; alpha-amylase inhibitors; beta-3 adrenoceptor agonists; ob gene products (Eli Lilly & Co.); SWR-0342-SA; SWR-0335; SP-18904; oral insulin mimetics; obesity therapeutics (7TM Pharma); beta-hydroxysteroid dehydrogenase (HSD) inhibitors; QRX-431; E-6776; M-450; melanocortin-4 antagonists; melanocortin 4 receptor agonists; obesity therapeutics (CuraGen); leptin mimetics; A-74498; second-generation leptin; NBI-103; CL-314698; CP-114271; beta-3 adrenoceptor agonists; NMI-8739; UCL-1283; BMS-192548; CP-94253; PD-160170; nicotinic agonist; LG-100754; SB-226552; LY-355124; CKD-711; L-751250; PPAR inhibitors; G-protein therapeutics; obesity therapy (Amylin Pharmaceuticals Inc.); BW-1229; monoclonal antibody (ObeSys/CAT); L-742791; (S)-sibutramine; MBU-23; YM-268; BTS-78050; tubby-like protein genes; genomics (eating disorders; Allelix/Lilly); MS-706; GI-264879A; GW-409890; FR-79620 analogs; obesity therapy (Hybrigenics SA); ICI-198157; ESP-A; 5-HT2C agonists; PD-170292; AIT-202; LG-100641; GI-181771; anti-obesity therapeutics (Genzyme); leptin modulator; GHRH mimetics; obesity therapy (Yamanouchi Pharmaceutical Co. Ltd.); SB-251023; CP-331684; BIBO-3304; 53holestene-3-ones; LY-362884; BRL-48962; PY-1 antagonists; A-71378;®-didesmethylsibutramine; obesity therapeutics (Bristol-Myers Squibb Co.); obesity therapeutics (Ligand Pharmaceuticals Inc.); LY-226936; NPY antagonists; CCK-A agonists; FPL-14294; PD-145942; ZA-7114; CL-316243; SR-58878; R-1065; BDBP-3226; HP-228; talibegron; FR-165914; AZM-008; AZM-016; AZM-120; AZM-090; AZM-131; AZM-132; AZM-134; AZM-127; AZM-083; AZM-1 15; AZM-140; vomeropherin; BMS-187257; D-3800; gene discovery (Axys/Glaxo); BRL-26830A; SX-013; ERR modulators; adipsin; AC-253; A-71623; A-68552; BMS-210285; TAK-677; MPV-1743; obesity therapeutics (Modex); GI-248573; exopipam; SSR-125180; obesity therapeutics (Melacure Therapeutics AB); BRL-35135; SR-146131; P-57; CGP-71583A; RF-1051; BMS-196085; manifaxine; DMNJ (Korea Research Institute of Bioscience and Biotechnology); BVT-5182; LY-255582; SNX-024; galanin antagonists; neurokinin-3 antagonists; dexfenfluramine; mazindol; diethylpropion; phendimetrazine; benzphetamine; amfebutmone; sertraline; AOD-9604; ATL-062; BVT-933; GT389-255; SLV319; HE-2500; PEG-axokine; L-796568; and ABT-239; rimonabant, sibutramine, orlistat, PYY or an analog thereof, CB-1 antagonist, leptin, phentermine, and exendin analogs; GPR1 19 agonists (e.g., anandamide; AR-231, 453; MBX-2982; Oleoylethanolamide; PSN-365,963; PSN-632,408; palmitoylethanolamide); GPR120 agonists; and GPR 40 agonists.

In some embodiments, one or more immunomodulators or a composition as described herein can be used (for example, administered or ingested) in combination with an antibiotic for treatment, prevention, maintenance, or prophylaxis of a disease or condition described herein. For example, Azithromycin, phenoxymethylpenicillin, dicloxacillin, amoxicillin with clavulanic acid, ampicillin, nafcillin, oxacillin, penicillin V, penicillin G, doxycycline, minocycline, sarecycline, erythromycin, clarithromycin, fidaxomicin, roxithromycin, ciprofloxacin, ofloxacin, levofloxacin, moxifloxacin, sulfamethoxazole with trimethoprim, sulfasalazine, sulfacetamide, sulfadiazine silver, vancomycin, dalbavancin, oritavancin, and telavancin.

### EXAMPLES

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. Changes therein and other uses which are encompassed within the spirit of the disclosure as defined by the scope of the claims will occur to those skilled in the art.

### Example 1

In this example, compounds described herein were tested to determine the effects of TNFα on barrier function and to analyze supernatant and cell lysate for subsequent analysis.

### Reagents

Cell Information: Donor 5 Transverse Colon, HISC-0033, Passage 10. Plated onto 4×96-well RepliGut® transwell plates coated with thin hydrogel coating. 2× RepliGut® transwell plates had 1.0 μm pore size (Corning 3380) and 2× RepliGut® transwell plates had 0.4 μm pore size (Corning 7369).

Compound Information: TNFα was supplied by Allis Biosystems and reconstituted according to the manufacturer's instructions. All other compounds were provided by Brightseed in DMSO (Table 1). Stocks were created for each treatment (Table 2) so that the final concentration of an individual compound in the culture media was 0.1% media volume for all compounds.

TABLE 1

| Reagent | Supplier | Solvent | Concentration | |
|---|---|---|---|---|
| Compound 1 | Brightseed | DMSO | 40 | mM |
| Compound 2 | Brightseed | DMSO | 40 | mM |
| Compound 2-1 | Brightseed | DMSO | 40 | mM |
| DMSO | Sigma (Cat#D8418) | N/A | N/A | |
| TNFα | R&D Systems | 0.5% BSA | 100 | μg/ml |
| | (Cat#210-TA-020) | in PBS | | |

Compound 1 - N-trans-caffeoyltyramine
Compound 2 - N-trans-feruloyltyramine
Compound 2-1 - N-trans-feruloyltyramine + N-trans-caffeoyltyramine as a 2.2:1 ratio of N-trans-feruloyltyramine to N-trans-caffeoyltyramine

Proliferative human transverse colon epithelial cells were plated in Altis Expansion media using Altis-established protocols onto 4×96-well RepliGut® transwell plates coated with a thin layer of hydrogel. Cultures were monitored every day using a bright field microscope. Transverse colon epithelial monolayers became confluent on Day 4, at which point media was changed to Altis Differentiation media. The cells were cultured for an additional 2 days. Brightseed compounds and TNFα, listed in Table 2, were diluted in Altis Differentiation media, mixed well, and added to apical and basal compartment of transwell cultures at t=0 hours. All conditions were performed in triplicate. Cells were treated with compounds for a total of 48 hours.

TABLE 2

| Treatment* | Dose 1 | Dose 2 | Dose 3 |
|---|---|---|---|
| No Treatment | N/A | — | — |
| Vehicle (DMSO) | 0.1% | — | — |
| Compound 1 | 10 μM | 20 μM | 40 μM |
| Compound 2 | 10 μM | 20 μM | 40 μM |
| Compound 2-1 | 10 μM + | 20 μM + | 40 μM + |
| | 10 μM | 20 μM | 40 μM |

*Treatment above were given with +/−10, 30, or 100 ng/ml TNFα

For this initial experiment, only treatments and deliverables for “Plate Set 2” were run to determine the optimal transwell membrane pore size for the subsequent experimental plates. It was suggested to run both a 0.4 μm and 1.0 μm pore size transwell membrane plates to obtain data to further investigate imaging capabilities of immunofluorescent staining on both transwell membrane types.

### Transepithelial Electrical Resistance (TEER)

Transepithelial electrical resistance was measured in all transwell cultures at t=0 hours prior to the addition of compounds to determine baseline TEER using an Epithelial Volt/Ohm Meter (World Precision Instruments, EVOM2) and STX100C96 Electrode. TEER was also measured at t=24 hours, t=45 hours, and t=48 hours. The surface area of a 96-well transwell is 0.143 cm² per well. Corrected TEER was calculated using the formula below.

(Raw measured TEER of sample transwell−Raw measured TEER of blank transwell)×Surface Area

### Supernatant Collection

After the TEER timepoint at t=48 hours, apical and basal supernatants were transferred to a 96-well polypropylene storage microplate (Thermo Fisher, Cat #267334), sealed with Sterile AlumaSeal II Sealing Film (Genesee, Cat #12-531), and stored at −80° C. Supernatant collection plate layouts are the same as the treatment plate layouts.

### Permeability Assay

After collection of supernatants, a permeability assay was run on all transwells. Forty-five hours after the addition of compounds, 75 μL of 0.5 mg/mL 40 kd M.W. FITC-Dextran (Sigma, Cat #FD40S) solution made in Altis Differentiation media was placed in the apical compartment of each transwell and 210 μL of Altis Differentiation media was placed in the basal compartment of each transwell. Transwell cultures were incubated for 3 hours at 37° C. with 5% CO2 to allow for the transfer of FITC-Deμxtran into the basal compartment. Two aliquots of 90 μL of media were collected from the basal compartment of each transwell and fluorescence was measured (Ex: 485, Em: 535) for each sample (n=2 samples/well) using a Synergy H1 Plate Reader.

“Raw fluorescence (RF) of media” was obtained by measuring Altis DM without FITC-Dextran to determine background fluorescence of the media. “RF of no cell control,” was obtained from basal compartment media of hydrogel-coated transwells that were not seeded with epithelial cells to normalize permeability measurements. “Relative Permeability (% of No Cell Control)” was calculated using the formula below.

$$( ( RF of sample - RF of media ) / ( RF of no cell control - RF of media ) ) \times 100$$

### Statistical Analysis

Statistical analysis was performed using GraphPad Prism software version 9.4.1 (GraphPad Inc., La Jolla, CA, USA). Two-way ANOVA of means from n=3 samples per group was performed to measure simple effects within treatment groups. Tukey's range test was performed to correct for multiple comparisons. Differences between the groups were considered significant at p<0.05.

### Results

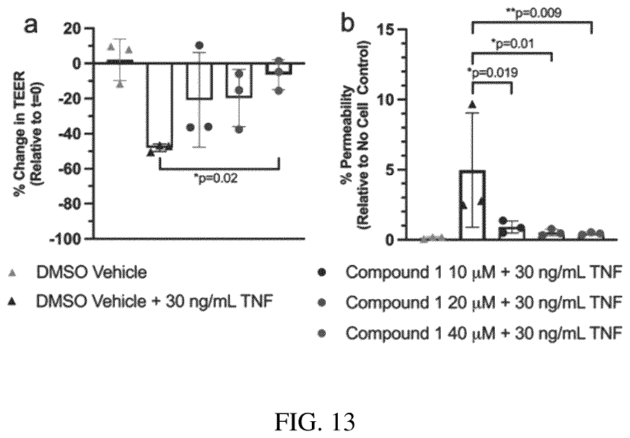

Administration of NCT improved the change in TEER as a result of TNF administration in a dose-dependent manner after 48 hours, with increasing improvements from 10 to 20 to 40 μM (FIG. 13 panel (a)). Values shown are relative to t=0. As little as 10 μM NCT resulted in improvements in % permeability that nearly countered the effects of TNF administration (FIG. 13 panel (b)). Higher doses of NCT (20 μM and 40 μM) exhibited a similar improvement to the 10 μM NCT dose.

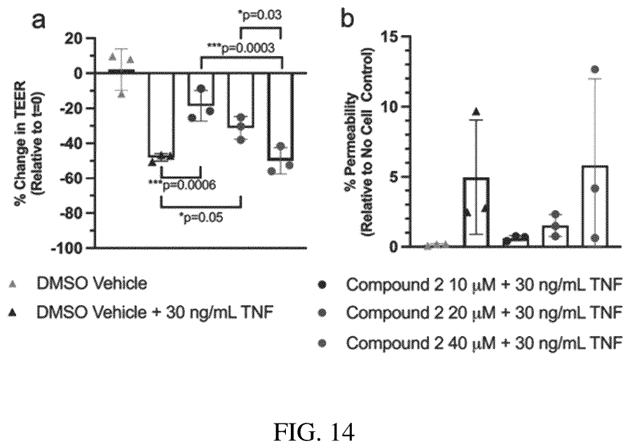

Administration of NFT improved TEER at 10 μM; however, increasing the concentration of NFT to 20 and 40 μM did not show the same dose-dependent effect that NCT exerted (FIG. 14 panel (a)). Similarly, the lowest dose of NFT improved % permeability to the extent that the effects of TNF were nearly completely mitigated (FIG. 14 panel (b)). As the dose of the NFT increased, so did the variability of the % permeability such that a dose-dependent effect was not observed.

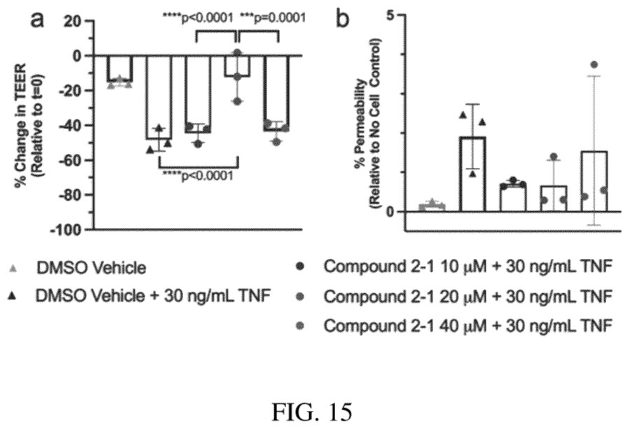

When administration in combination, NCT and NFT exhibited the greatest beneficial effect at 20 μM with respect to reversing TEER changes due to TNF (FIG. 15 panel (a)). The lowest doses (10 μM, 20 μM) had the greatest benefit on % permeability, while the highest dose (40 μM) did not yield a substantive change in % permeability compared to control (FIG. 15 panel (b)).

The data presented in this contribution show that NCT, and, to a lesser extent, NFT, positively affect gut barrier function in a state of elevated inflammation. This example confirms that NCT and NFT improve markers of gut barrier function during a state of elevated inflammation in vitro. These two bioactive compounds have the potential to exert profound effects on human health. Several pre-clinical experiments, including the data presented in this example have demonstrated a potential for NCT to exert a HNF4a-mediated benefit on intestinal function. Additionally, HNF4α has a well-documented effect on epithelial cell transcription activity and the related transcriptome (Ntunzwenimana et al., 2021). In patients with ulcerative colitis, HNF4α is significantly downregulated compared to controls, suggesting a role in the disease state (Vancamelbeke et al., 2017). Thus, the case for HNF4a's role in gut inflammation and overall gut health is evident. Without wishing to be bound by theory, NCT and NFT have the potential to mediate gut health and gut barrier integrity via HNF4a. The inclusion of bioactive compounds in dietary recommendations can inform a new, national standard for nutrition and dietary guidelines and enable a new food-as-medicine paradigm for preventative health.

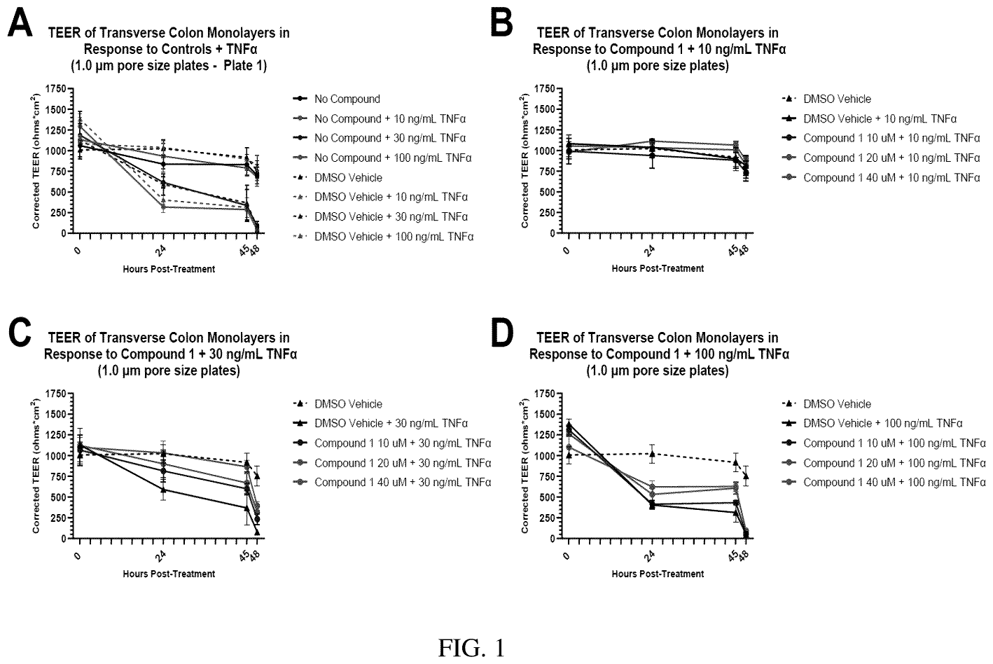

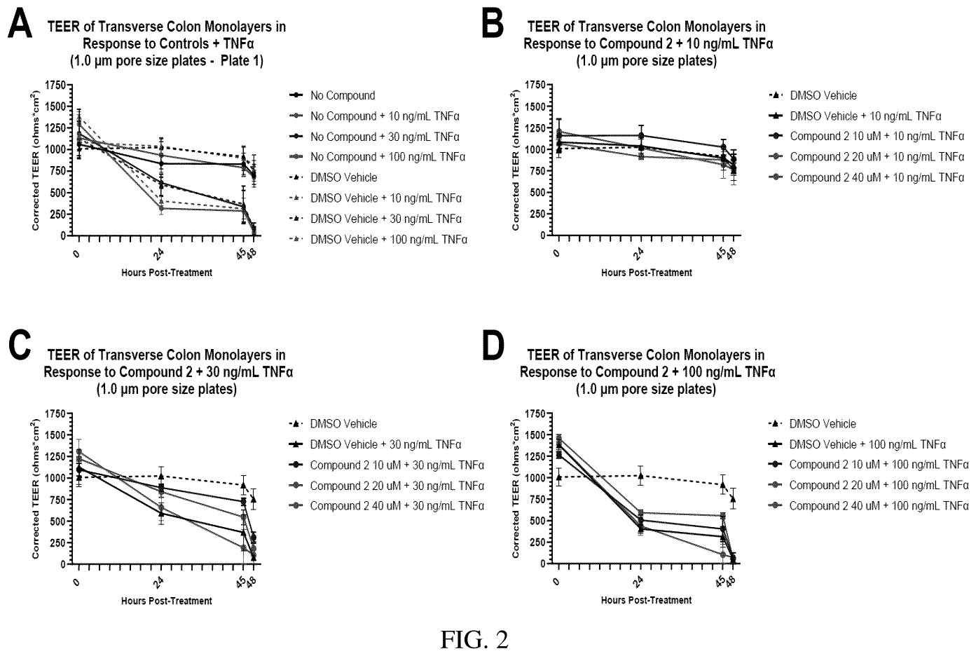

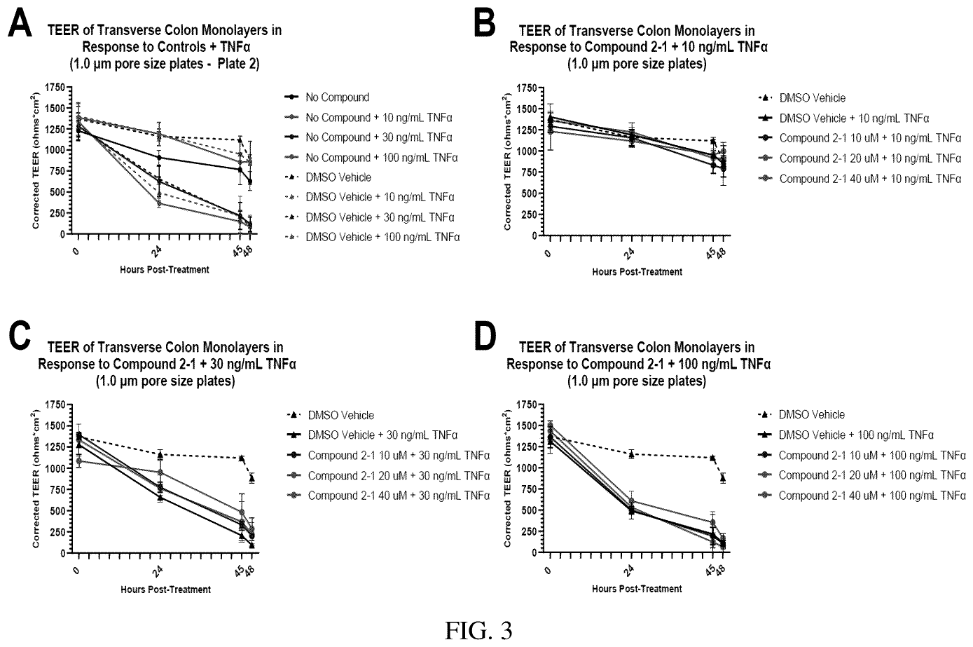

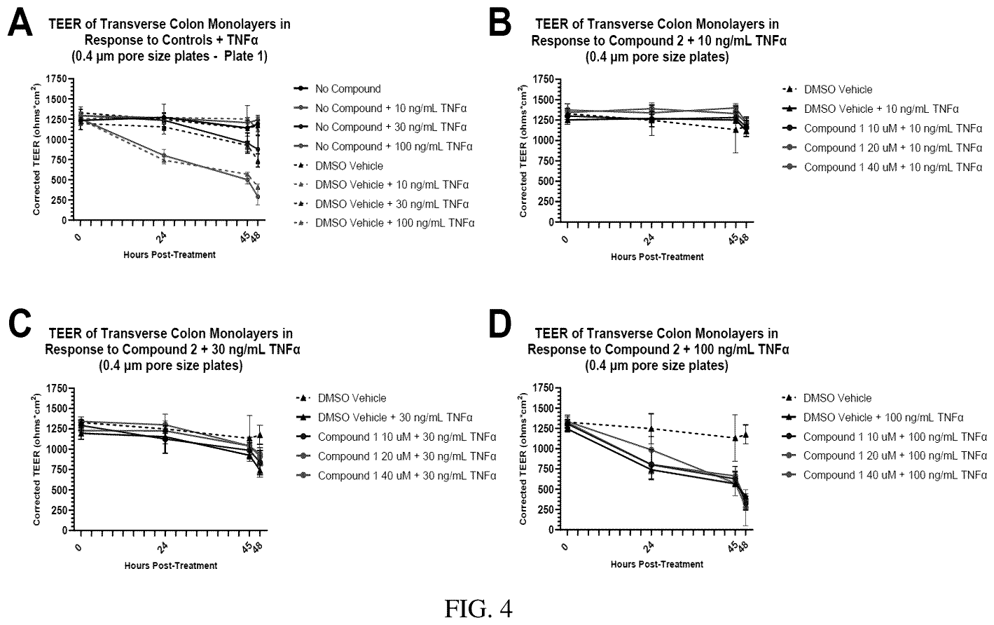

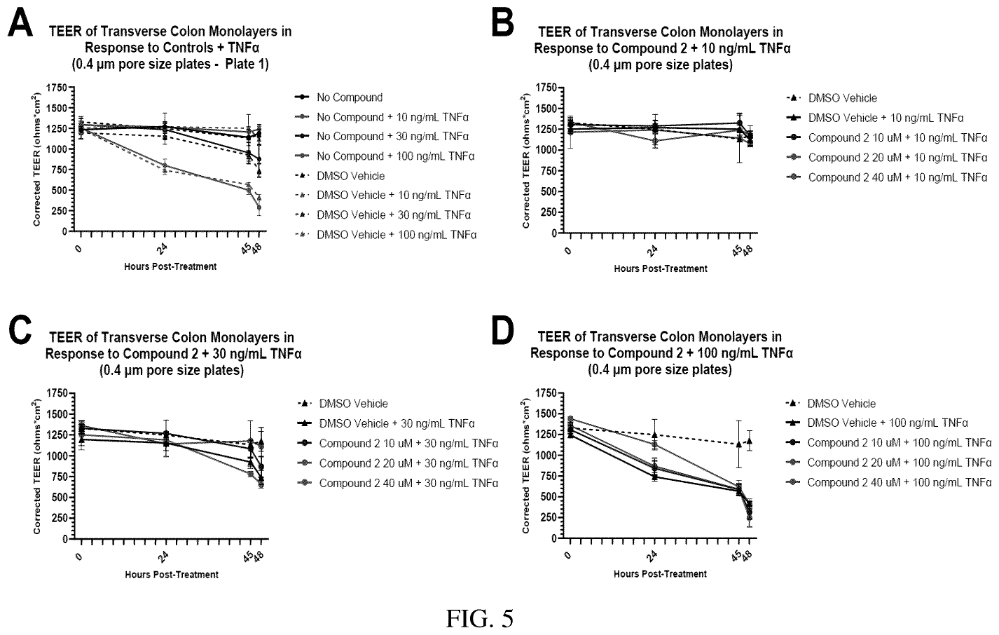

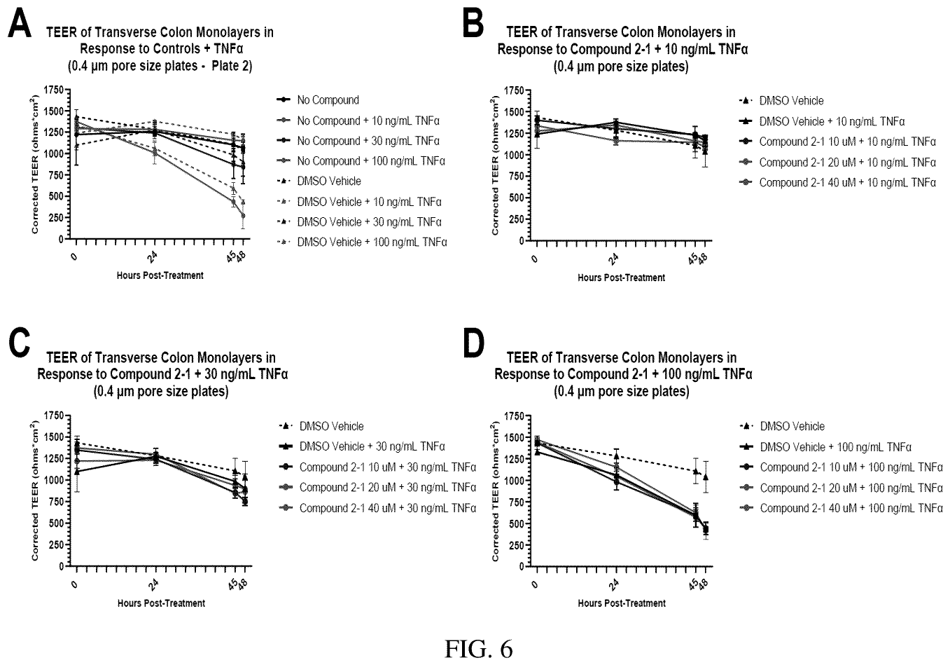

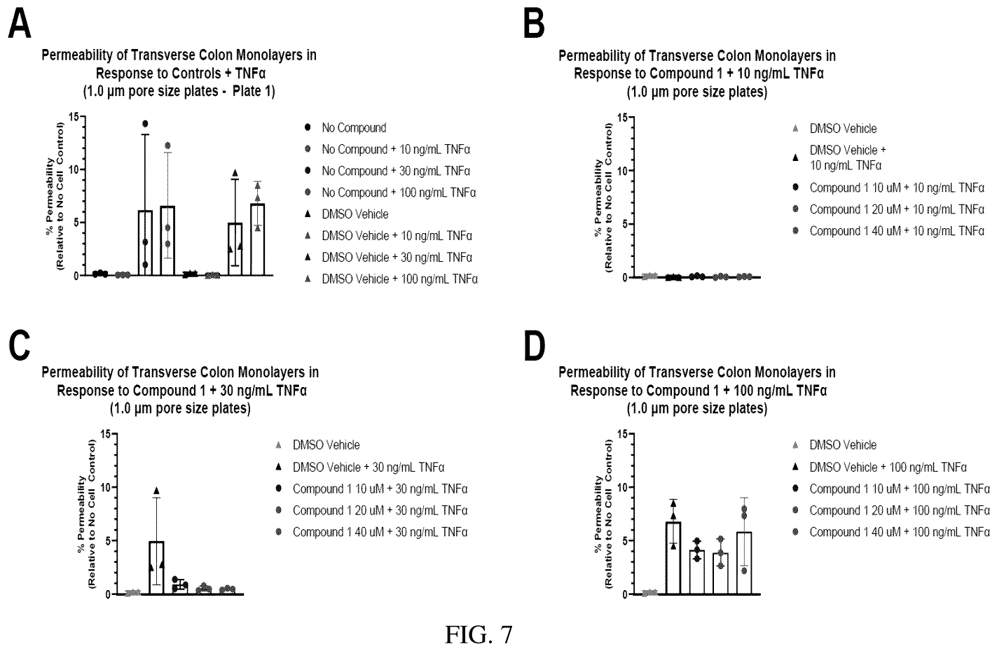

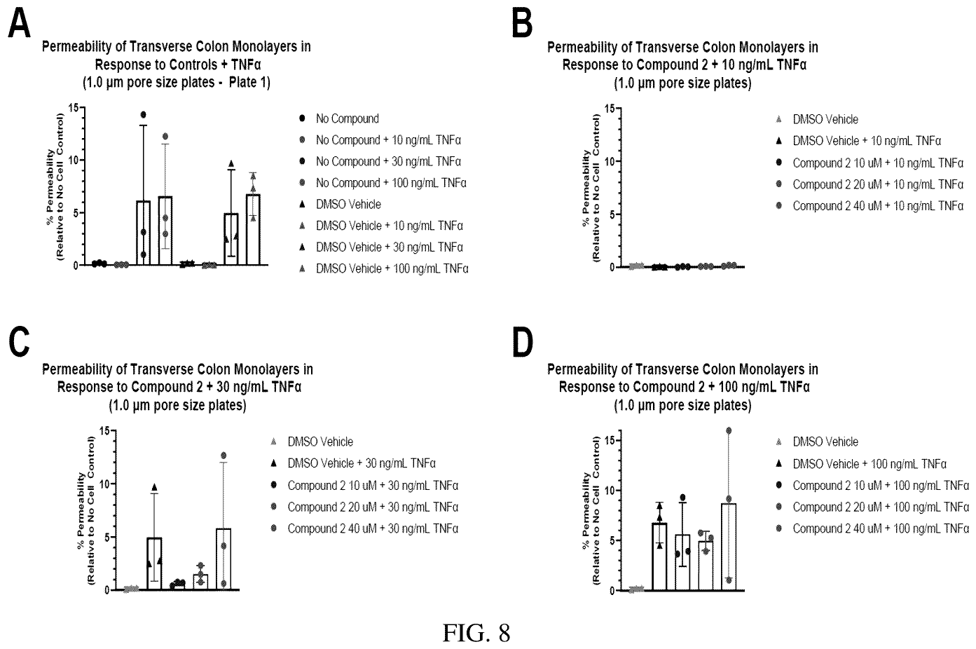

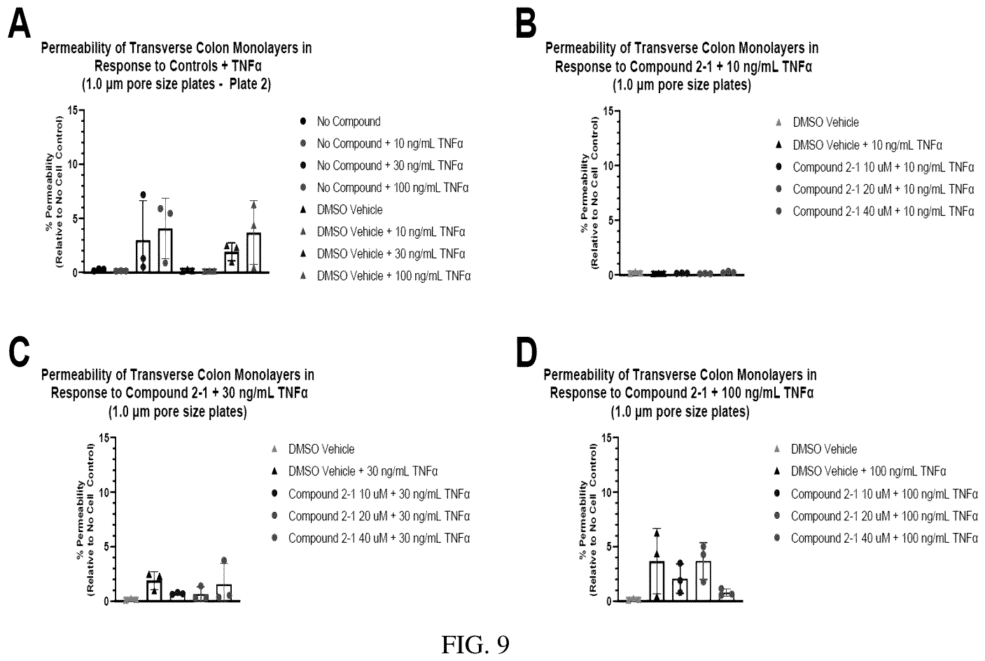

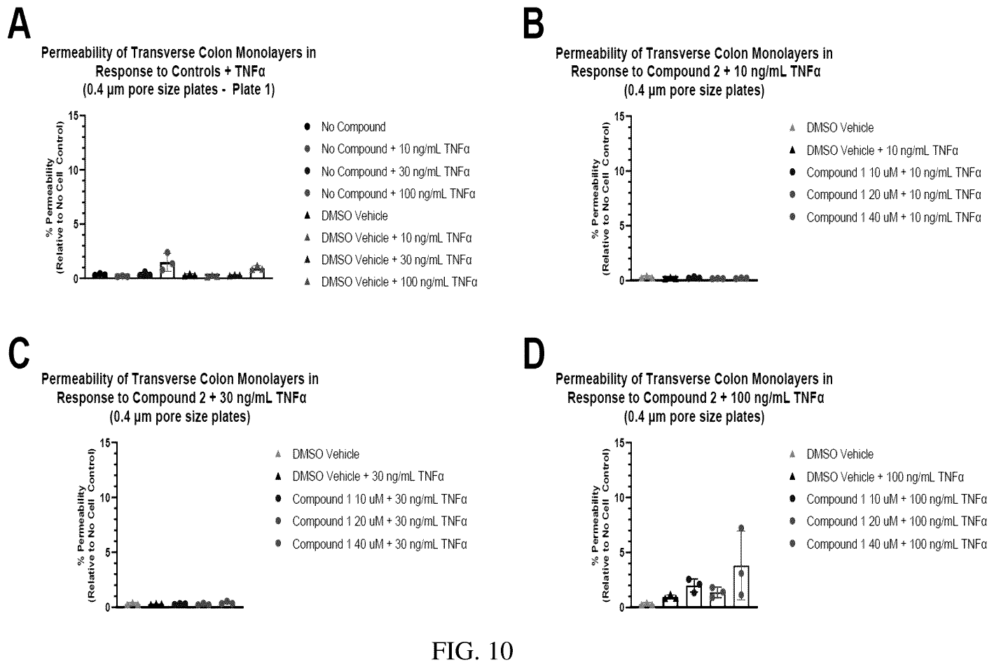

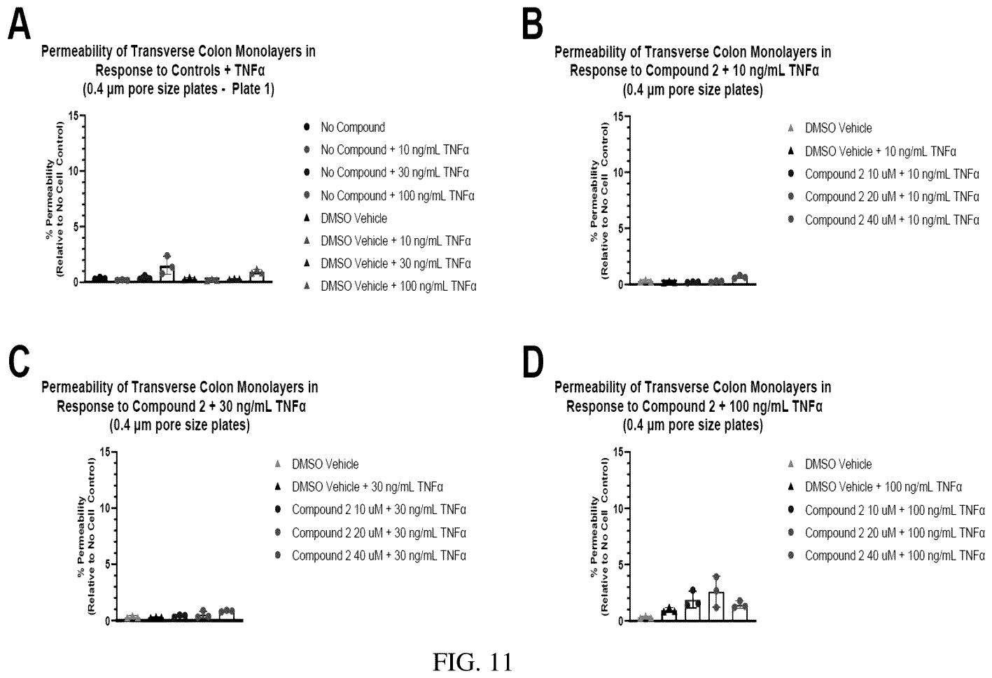

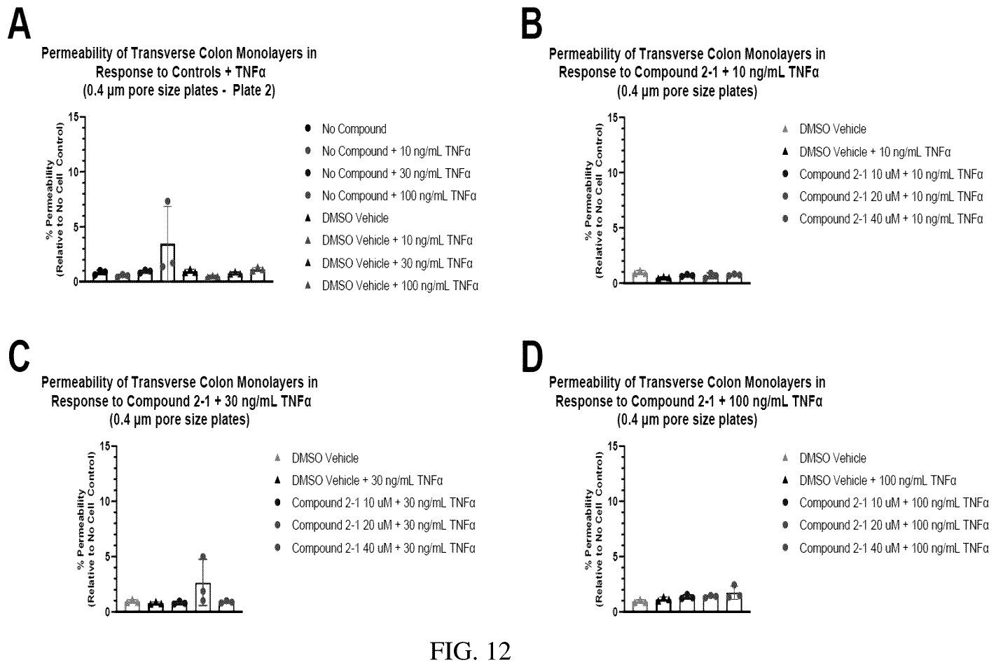

The data from this example is further illustrated in FIGS. 1-12. It was determined that Compound 1 had the greatest effect, followed by Compound 2-1, and Compound 2. It was also determined that the optimal concentration of the study for Compound 1 was 40 μM while Compound 2-1 was between 20 and 40 μM and Compound 2 was between 10 and 20 μM. Thus, a significant decrease in TEER was observed with increased addition of TNFα in the no treatment and 0.1% DMSO transwells indicating increased permeability. Addition of N-trans-caffeoyltyramine and N-trans-feruloyltyramine demonstrated a dose-dependent and statistically significant reversal of this decrease. A significant HNF4α expression was found in the colonic cells. Accordingly, novel bioactives N-trans-caffeoyltyramine and N-trans-feruloyltyramine demonstrated a significant improvement in TEER and a decrease in intestinal permeability.

### Example 2

In this example, a randomized, double-blind, placebo-controlled, parallel study to investigate the effects on gastrointestinal barrier function of compounds described herein.

### Background

HNF4α is a signaling compound, known as a nuclear transcription factor that plays a role in regulating metabolism in the liver. It has also been identified in the intestine, pancreas, and kidney, where it serves other functions. HNF4α influences intestinal permeability within the intestine and is associated with inflammatory bowel diseases, although its role is unclear. HNF4α has been documented to exert numerous effects, specifically on gut barrier and intestinal function. Specifically, HNF4α impacts mucin production, a key component of the gut barrier. It also regulates the expression of tight junction proteins and Paneth cell differentiation. The direct interaction of NCT and NFT with HNF4α makes NCT and NFT potent agonists of HNF4a.

As described in Example 1, primary intestinal epithelial tissue derived from adult human stem cells were used to assay permeability & transport, cytokine secretion, toxicity, and gene/protein expression. A human stem cell based in vitro model using human transverse colon epithelial cells was exposed to the proinflammatory cytokine TNFα, resulting in increased intestinal permeability/decrease in transepithelial electrical resistance (TEER). Co-administration of NCT and NFT demonstrated a dose-dependent and statistically significant reversal of impaired TEER and intestinal permeability. NCT and NFT demonstrated a physiologically relevant reversal of impaired gut barrier function in inflammation via significant improvement in TEER and percent permeability.

The safety of NCT/NFT has been evaluated within the matrices of a hemp hull fiber ingredient. The safety of the phenolic content, including NCT and NFT, was based on the history of hemp consumption, publicly available literature, and unpublished data generated showing no adverse events in 5 studies in male rodents receiving 400 to 600 mg/kg bw/d for 10 d to 4 mo and in a human study with adult men (18-42 y) receiving 10 mg/d for 7 d. Considering the maximum permissible phenolic spec of 100 mg GAE/g and that NCT and NFT compose approximately 4.5% of the phenolic fraction in hemp hulls, a cumulative 90th percentile estimated daily intake for adult males of NCT and NFT from hemp hull fiber of 176 mg/d is permitted under the GRAS assessment (Leonard et al., 2021). Hemp hull fiber may contain trace levels of THC and CBD comparable to levels reported in FDA notified GRAS for the hemp ingredients mentioned above and found by the Agency to pose no safety concerns. One aim of the current study is to examine the effect of a hemp hull extract standardized to contain 10-15% NCT and NFT. The dose levels of NCT and NFT evaluated based on the safety conclusion reached in the GRAS for the hemp hull fiber, can reasonably be concluded to pose no safety concern to study participants.

### Objective

An objective of this clinical study will be to investigate the effects of a composition described herein supplementation for 6 weeks on parameters of gastrointestinal (GI) health in otherwise generally healthy adults with risk factors for increased GI permeability. The primary hypothesis is that supplementation with the composition as described herein will decrease small intestinal permeability compared to placebo. Lactulose is a disaccharide that is not typically absorbed by the intestine, whereas the monosaccharide mannitol does undergo absorption. The 2-h urinary LMR ratio serves as a measure of small intestinal permeability, with a higher ratio indicating more lactulose in urine and, therefore greater small intestinal permeability. This study will also test for the contributions of supplementation on distal GI permeability (2 to 8 h), GI microbiome composition profiles, bowel habits, safety assessments (blood chemistry/hematology, vital signs, and adverse events), and common GI symptoms such as gas/flatulence and abdominal bloating that are typically associated with increased GI permeability.

### Study Design

A randomized, double-blind, placebo-controlled, three-arm parallel design consisting of one screening visit (Vist 1, Day-7) and three study visits (Days 0, 21, and 42) with enrollment occurring in two stages including an interim analysis once 50% of the study participants are enrolled.

Subjects will be instructed to fast (≥10 h) prior to arriving for their screening visit on Day −7. Those who are taking exclusionary products (Appendix 1) will be instructed to stop taking these products, and Visits 1 (Day −7) will be scheduled to allow for the appropriate washout period.

At Visit 1 (Day −7), subjects will arrive at the clinic in a fasting state (≥10 h). After subjects provide voluntary informed consent, subjects will undergo a medical history, prior and current medication/supplement use, inclusion and exclusion criteria assessments, and last menses query, if applicable. Additionally, height, body weight, and vital signs will be measured, and BMI will be calculated. An in-clinic urine pregnancy test will be performed on all female subjects <60 years old. Blood samples will be collected for chemistry, hematology, and hsCRP analyses.

Subjects will be dispensed a 3-d Diet Record and a Bowel Habits Diary with instructions to record all foods and beverages consumed, and information about all bowel movements, respectively, over 3 consecutive days immediately prior to Visit 2 (Day 0). Subjects will also be dispensed a stool collection kit with instructions to collect 3 separate fecal samples from a single bowel movement during the same 3 days of the Diet Record and Bowel Habits Diary are completed prior to Visit 2 (Day 0). Prior to departing the study center, subjects will also be instructed to maintain physical activity and habitual diet as much as possible prior to departing the study center, subjects will also be instructed to maintain physical activity and habitual diet as much as possible while limiting the introduction of new foods to their habitual diet and foods that are known to cause GI distress in the individual subjects, and abstaining from supplements, beverages, or food products with live probiotics (e.g., yogurt, kombucha) prior to Visit 2 (Day 0). Lastly, subjects will also be reminded to abstain from vigorous exercise, NSAIDs, and alcohol for 48 h, and fast (≥10 h; water only) prior to Visit 2 (Day 0).

At Visit 2 (Day 0), subjects will arrive at the clinic in a fasting state (≥10 h) to undergo clinic visit procedures (concomitant medication/supplement use, assess inclusion/exclusion criteria, body weight, vital signs measurements, and last menses query, if applicable) and adverse event (AE) assessment. Subjects will be queried about compliance with study instructions. Subjects will also be randomly assigned to one of three study product sequences. The 3-d Diet Record and Bowel Habits Diary will be collected and reviewed. Three fecal samples will be collected, and subjects will be administered a GITQ 7-day recall. Blood samples will be collected for chemistry, hematology, and hsCRP. Additional plasma samples will be collected for possible future analysis of non-genetic biomarkers of GI permeability and inflammation. Subjects will then complete a GI permeability test where they will ingest two sugar probes (1 g 13 C mannitol, and 5 g lactulose) in ˜240 mL water. The time of sugar probe consumption will be t=0 h. Following sugar probe consumption, subjects will collect all urine in-clinic for 8 h, with urine collected from 0 to 2 h in one container and from 2:01 h to 8 h in a second container. Urine collection containers will remain at room temperature throughout the 8 h collection period, and subjects will be allowed to consume water ad libitum. A standard snack will be provided at t=2 h and a standard lunch will be provided at t=4 h. Subjects will consume the lunch within 30 min and will be instructed to eat until comfortably full. Lunch and water consumption will be recorded, and subjects will be required to replicate at Visit 4 (Day 42). Study products will be dispensed, a standard snack will be administered, and the first capsule(s) will be consumed in-clinic. An electronic Study Product Log will begin documenting consumption at this visit and will be administered electronically daily throughout the remainder of the study period.

Prior to departure from the study center, AEs will be assessed, and study instructions will be provided, including 1) maintenance of physical activity and habitual diet as much as possible except for consumption of study products, 2) minimizing the introduction of new foods to their habitual diet and foods that are known to cause GI distress in the individual subjects, and 3) abstaining from supplements, beverages, or food products with live probiotics (e.g., yogurt, kombucha) throughout the study. Lastly, subjects will also be reminded to abstain from vigorous exercise, NSAIDs, and alcohol for 48 h, and fast (≥10 h; water only) prior to Visit 3 (Day 21).

At Visit 3 (Day 21), subjects will arrive at the clinic in a fasting state (≥10 h) and undergo clinic visit procedures, including concomitant medication/supplement use, review inclusion/exclusion criteria, body weight, and vital signs measurements, and last menses query, (if applicable) and AE assessment. Subjects will be queried about compliance with study instructions. Compliance with the study product consumption will be assessed by counting returned unused study, and the electronic Study Product Log will be reviewed. Study products for that day will be consumed in-clinic, and new study products will be dispensed for the remainder of the study period. Blood samples will be collected for chemistry, hematology, and hsCRP analyses. Subjects will be dispensed a blank 3-d Diet Record along with a photocopy of the diet record completed 24 h prior to Visit 2 in order to replicate food/beverage intake for 24 h prior to Visit 4, and a Bowel Habits Diary with instructions to record and foods and beverages consumed and record information about all bowel movements over 3 consecutive days immediately prior to Visit 4 (Day 42). Subjects will also be dispensed a stool collection kit with instructions to collect 3 separate fecal samples from a single bowel movement during the same 3 days of 3-d Diet Record and Bowel Habits Diary completion prior to Visit 4 (Day 42). Prior to departure from the study center, study instructions will be provided, including: 1) maintenance of physical activity and habitual diet as much as possible except for consumption of study products, 2) minimizing the introduction of new foods to their habitual diet and foods that are known to cause GI distress in the individual subjects, and 3) abstaining from supplements, beverages, or food products with live probiotics (e.g., yogurt, kombucha) throughout the study. Lastly, subjects will also be reminded to abstain from vigorous exercise, NSAIDs, and alcohol for 48 h, and fast (≥10 h; water only) prior to Visit 4 (Day 42).

At Visit 4 (Day 42), subjects will arrive at the clinic in a fasting state (≥10 h) and undergo clinic visit procedures, including concomitant medication/supplement use, review inclusion/exclusion criteria, body weight, and vital signs measurements, and last menses query, (if applicable) and AE assessment. Subjects will be queried about compliance with study instructions. The 3-d Diet Record will be collected and reviewed, and the last 24 h of food and beverage intake will be compared to the 24 h prior to Visit 2 to ensure consistency. The Bowel habits diary will be collected and reviewed. Three fecal samples will be collected, and subjects will be administered a GITQ 7-day recall. Blood samples will be collected for chemistry, hematology, and hsCRP. Additional plasma samples will be collected for possible future analysis of non-genetic biomarkers of GI permeability and inflammation. Compliance with study product consumption will be assessed by counting returned unused study products, and the electronic Study Product Log will be reviewed. Subjects will then complete the GI permeability test with the same visit procedures and study instructions described at Visit 2.

TABLE 3

| | Flow Chart (N = 126) | | | |
|---|---|---|---|---|
| | Screen | Intervention | | |
| | Visit¹ | | | |
| | 1 | 2 | 3 | 4 |
| | Day | | | |
| | −7 | 0 | 21 | 42 |
| Informed Consent/HIPAA² | X | | | |
| Medical History | X | | | |
| Clinic Visit ³ | X | X | X | X |
| In-clinic Urine Pregnancy Test ⁴ | X | | | |
| Chemistry Profile ⁵ | X | | X | X |
| Hematology Profile ⁶ | X | | X | X |
| Plasma hsCRP ⁷ | X | | X | X |
| Study Instructions/Query ⁸ | X | X | X | X |
| Dispense Blank 3-d Diet Record ⁹ | X | | X | |
| Dispense Copy of 24 h Diet Record prior to Visit 2 for Replication 24 h | | | X | |
| prior to Visit 4 ¹⁰ | | | | |
| Dispense Bowel Habits Diary ¹¹ | X | | X | |
| Dispense Stool Collection Kit ¹² | X | | X | |
| Collect/Analyze 3-d Diet Record ⁹ | | X | | X |
| Collect Bowel Habits Diary ¹¹ | | X | | X |
| Collect Fecal Sample - GI Microbiome Analysis ¹² | | X | | X |
| Collect Fecal Samples - Biomarkers of GI Permeability ¹² | | X | | X |
| Administer GITQ 7-day Recall ¹³ | | X | | X |
| Plasma - Biomarkers of GI Permeability ¹⁴ | | X | | X |
| Plasma - Biomarkers of Inflammation ¹⁵ | | X | | X |
| Administer Lactulose/Mannitol GI Permeability Test ¹⁶ | | X | | X |
| Provide Standard Lunch | | X | | X |
| Provide Standard Snack | | X | | X |
| Randomization | | X | | |
| Dispense Study Product and Administer e-Study Product Log ¹⁷ | | X | X | |
| Collect Unused Study Product and Review e-Study Product Log | | | X | X |
| Adverse Events Assessment ¹⁸ | | X | X | X |

Footnotes:
¹A window of +7 d will be allowed between visits 1 and 2 (Days −7 and 0). A window of ±2 d is allowed for Visit 3, anchored to Visit 2. A window of ±2 d is allowed for Visit 4, anchored to Visit 2.
²HIPAA = Health Insurance Portability and Accountability Act authorization to disclose protected health information. The signed document authorizes the use and disclosure of the subject's Protected Health Information by the Investigator and by those who need that information for the study.
³ Clinic visit procedures include measurement of height (Visit 1 only), vital signs (resting blood pressure and heart rate), and body weight, BMI calculation (Visit 1 only), evaluation of inclusion and exclusion criteria, concomitant medication/supplement use, and last menses query (where applicable).
⁴ In-clinic urine pregnancy tests will be completed for all women <60 y.
⁵ The fasting (≥10 h) chemistry profile will include albumin, alkaline phosphatase, total bilirubin, calcium, chloride, creatinine, blood urea nitrogen (BUN), potassium, aspartate aminotransferase (AST), alanine aminotransferase (ALT), sodium, total protein, carbon dioxide (CO₂), osmolality, and glucose.
⁶ The fasting (≥10 h) hematology profile will include white blood cell count (WBC), red blood cell count (RBC), hemoglobin concentration, hematocrit (as volume percent), mean cell volume (MCV), mean cell hemoglobin (MCH), mean cell hemoglobin concentration (MCHC), neutrophils, lymphocytes, monocytes, eosinophils, basophils, and platelet count.
⁷ hsCRP at screening Visit 1 will be used to stratify participants with values <2.0 and ≥2.0 mg/dL among the three test conditions.
⁸ Study instructions will be provided, including Maintenance of physical activity and habitual diet as much as possible, except for consumption of study products. Minimize introducing new foods to the habitual diet and foods known to cause GI distress in the individual subjects. Abstain from supplements, beverages, or food products with live probiotics (e.g., yogurt, kombucha) throughout the study.
⁹ Subjects will be provided with a blank 3-d Diet Record to record all foods and beverages consumed during the 3 days immediately prior to Visits 2 and 4 (Days 0 and 42). Each Diet Record should be completed over the same 3-d period coinciding with the Bowel Habits Diary and stool specimen collection. The completed diet records will be reviewed for exclusionary foods/beverages and analyzed for nutrient intake.
¹⁰ A copy of the last day of the baseline 3 d diet record (i.e., the 24 h prior to Visit 2) will be dispensed for replication during the 24 h prior to Visit 4.
¹¹ The Bowel Habits Diary will provide information on stool frequency and consistency, straining and discomfort during bowel movements, and any sensation of incomplete evacuation. The Bowel Habits Diary will be dispensed at Visits 1 and 3 (Days −7 and 21) to be completed for 3 consecutive days concurrent with the 3-d Diet Record and stool specimen collection prior to Visits 2 and 4 (Days 0 and 42), respectively.
¹² A stool specimen collection kit will be dispensed for a single stool collection from one bowel movement during the 3 days immediately prior to Visits 2 and 4 (Days 0 and 42). Three separate fecal samples will be collected from the single stool specimen: one for analysis of the GI microbiome and two for non-genetic biomarkers of GI permeability (e.g., calprotectin, secretory IgA). Stool specimen and fecal sample collection instructions are provided.
¹³ GI symptoms, including gas/flatulence, nausea, vomiting, abdominal cramping, abdominal distention/bloating, borborygmus/stomach rumbling, burping, and reflux (heartburn), will be collected using GITQ at Visits 2 and 4 (Days 0 and 42). Subjects will be asked a series of questions regarding the presence and severity of GI symptoms occurring during the past 7-days, in which each response is ranked on a 4-point scale ranging from none-to-severe.
¹⁴ Six plasma samples will be collected for non-genetic biomarkers of GI permeability (e.g., I-FABP, LBP, soluble CD14, etc.)
¹⁵ Two plasma samples will be collected for non-genetic biomarkers of inflammation (e.g., TNF-α, IL-6, and/or IL-1β)
¹⁶ At visits 2 and 4, following an overnight fast, participants will ingest two sugar probes (5 g lactulose and 1 g ¹³C mannitol) in ~240 mL water. Urine will be collected in-clinic from 0 to 2 h and from 2:01 to 8 h. Total urine volume for each time period will be recorded and 1 mL aliquots will be frozen at −80° C. for later analysis and archiving. The urine collection containers can remain at room temperature throughout the 8 h collection period. Participants will be provided with a standard snack at t = 2 h, a standard lunch at t = 4 h, and a standard snack at t = 8 h.
¹⁷ First study product consumption will occur in-clinic at Visit 2 after the 8 h urine collection period. Additional study products will be dispensed for consumption at home with instructions to consume after waking and prior to the first eating occasion. An electronic study product log (Appendix 5) will be administered to subjects to record consumption of study products. Counting of unused study products will be the primary method used to assess compliance.
¹⁸ AE inquiries will occur with an open-ended question at Visits 2, 3, and 4 (Days 0, 21, and 42). AEs will be assessed at the beginning and end of Visits 2 and 4, and at the end of Visit 3.

### Study Sample

Each subject must meet all of the following inclusion criteria and none of the exclusion criteria in order to participate in this study.

### Inclusion Criteria

Male or female, 30-69 years of age, inclusive at Visit 1 (Day −7). BMI of ≥29.0 kg/m2 at Visit 1 (Day −7). Waist circumference >102 cm for men and >88 cm for women OR a family history of at least one first degree relative with a diagnosed GI disorder associated with increased GI permeability, such as Inflammatory Bowel Disease (Crohn's disease or ulcerative colitis). Non-user or former user (cessation 12 months) of tobacco or nicotine products (e.g., cigarette smoking, vaping, chewing tobacco) with no plans to begin use during the study period. Non-user or former user (cessation 6 months) of any marijuana or hemp products with no plans to begin use during the study period. Willing to maintain physical activity and exercise patterns, body weight, and habitual diet throughout the trial. Willing to refrain from exclusionary medications, supplements, and products throughout the study. No health conditions that would prevent him/her from fulfilling the study requirements as judged by the Clinical Investigator on the basis of medical history and routine laboratory test results. Understands the study procedures and signs forms providing informed consent to participate in the study and authorizes the release of relevant protected health information to the Clinical Investigator.

### Randomization

A randomization sequence will be prepared. Randomization will be 1:1:1 and stratified by hsCRP group. The sequence will be uploaded onto an eCRF (electronic case report form) platform (Medrio Inc, San Francisco, CA). When a subject is determined to be eligible for the study, a randomization number and associated blinded treatment arm will be assigned to the subject through the randomization module of the platform. The randomization number will be recorded in the subject's source documentation.

### Study Product

Placebo treatment: 2 capsules/d, Active low dose: 1 capsule/d+1 placebo capsule/d, and Active high dose: 2 capsules/d. Every day for 42 days, subjects will be instructed to consume their assigned study product after waking with water and at least 30 minutes before the first eating occasion. If a subject fails to consume the study product at that time (i.e., normally consumes before breakfast but forgot), he/she will be instructed to consume their assigned product with water at least 30 minutes prior to the next eating occasion (e.g., if missed prior to breakfast, consume study product a least 30 minutes before lunch). Subjects will be advised not to consume more than 2 capsules/d.

### Blinding

Subjects and study staff will remain blinded to the study product throughout the study. A set of sealed unblinding envelopes will be provided to the Clinical Investigator for use in an emergency situation where knowledge of the assignment is essential for the subject's immediate medical care. If unblinding is needed, the Clinical Investigator and study staff will open a sealed unblinding envelope to determine the subject's product assignment.

The blinding code of the study products must be broken only in exceptional circumstances, such as when knowledge of the study products is essential for treating a subject due to a Serious Adverse Event (SAE).

The Clinical Investigator or other representative will be contacted immediately if it is necessary to unblind the randomization sequence for a subject. Whenever possible, this should be done prior to such unblinding. The study Sponsor will also be notified should unblinding be required.

Documentation of the unblinding will be made in the source documents indicating the reason for the unblinding and the date and time that unblinding occurred.

### Clinic Visit Procedures

Clinic visit procedures (Visits 1, 2, 3, and 4; Days −7, 0, 21 and 42) include measurement of height (Visit 1 only), vital signs (resting blood pressure and heart rate), and body weight, BMI calculation (Visit 1 only), evaluation of inclusion and exclusion criteria, concomitant medication/supplement use, and last menses query (where applicable).

Standardized vital signs measurements assessed at each clinic visit will include resting blood pressure and heart rate measured using an automated blood pressure measurement device with an appropriately sized cuff (bladder within the cuff must encircle ≥80% of the arm) obtained after the subject has been sitting quietly for at least 5 minutes. Three measurements will be taken ˜three minutes apart (e.g., 0, 3, and 6 min), with the final two measurements averaged and the first discarded. Should elevated blood pressure occur at the screening visit (Visit 1, Day −7), the subject will be documented as a screen failure and a re-test will not be allowed.

### Laboratory Measurements

The procedures for all clinical laboratory measurements will be outlined in a laboratory instruction document. Laboratory parameters that are missing or have not been obtained must be entered in the eCRF as “not done.”

An in-clinic urine pregnancy test will be performed on all women <60 years old at Visit 1 (Day −7).

The following will be performed at Visits 1, 3, and 4 (Days −7, 21, and 42):

Fasting blood chemistry profile: Albumin, aspartate aminotransferase, alanine aminotransferase, alkaline phosphatase, total bilirubin, calcium, chloride, creatinine, blood urea nitrogen, potassium, sodium, total protein, carbon dioxide, osmolality, and glucose. These will be measured by Elmhurst Memorial Reference Laboratory (Elmhurst, IL). Fasting blood hematology: White blood cell count, red blood cell count, hemoglobin concentration, hematocrit (as volume percent), mean corpuscular volume, mean corpuscular hemoglobin concentration, neutrophils, lymphocytes, monocytes, eosinophils, basophils and platelet count. These will be measured by Elmhurst Memorial Reference Laboratory (Elmhurst, IL).

Fasting hsCRP will be measured by Elmhurst Memorial Reference Laboratory (Elmhurst, IL).

The following will be performed at Visits 2 and 4 (Days 0 and 42): GI permeability will be assessed using a two-sugar probe procedure. Two pooled urine samples (0-2 h and 2:01-8 h) will be collected over an 8 h period for subsequent analysis at the Immunochemical Core Lab at The Mayo Clinic (Rochester, MN). Biofortis is responsible for determining total urine volume in the 0-2 h and 2:01-8 h collection intervals and supplying a record of urine volumes and aliquots to the Core Laboratory. Urinary sugars will be analyzed by using established procedures in the Core Lab. A fecal sample will be stored at −80° C. in a DNA Genotek OMNIgene®⋅GUT tube at Biofortis for possible future microbiome composition (i.e., taxonomical profiles) and/or functional profile assessments at a laboratory to be designated. Briefly, genomic DNA will be extracted using an appropriately optimized, high-throughput stool extraction method. DNA will then be quantified and stored at appropriate temperatures until genomic library preparation begins. Genomic libraries will then be sequenced on an appropriate platform, and sequencing data will be used in bioinformatic analyses to facilitate the phylogenetic classification of bacteria/archaea with corresponding alpha-diversity and beta-diversity metrics determined. Functional bioinformatics analyses may include metabolic pathways and related approaches. Two fecal samples (˜100 g each) will be stored at −80° C. at Biofortis for possible future analysis of non-genetic biomarkers of GI permeability (e.g., calprotectin, secretory IgA).

Six plasma samples (˜0.5 mL each) will be stored at −80° C. at Biofortis for possible future analysis (and back-up samples) of non-genetic biomarkers of GI permeability (e.g., I-FABP, LBP, etc.).

Two plasma samples (˜0.5 mL each) will be stored at −80° C. at Biofortis for possible future analysis (and back-up sample) of non-genetic biomarkers of inflammation (e.g., TNF-α, IL-6, IL-1β, etc.).

### Study Instructions/Query

Prior to Visit 1 (Day −7), subjects will be instructed to abstain from exercise and alcohol (24 h), and fast (≥10 h, water only).

At Visit 1 (Day −7), subjects will be instructed to maintain physical activity and habitual diet as much as possible while limiting the introduction of new foods to their habitual diet and foods that are known to cause GI distress in the individual subjects, and abstaining from supplements, beverages, or food products with live probiotics (e.g., yogurt, kombucha) prior to Visit 2 (Day 0). Subjects will also be reminded to abstain from vigorous exercise, NSAIDs, and alcohol for 48 h, and fast (≥10 h; water only) prior to Visit 2 (Day 0).

At Visit 2 (Day 0), subjects will be instructed to maintain physical activity and habitual diet as much as possible while limiting the introduction of new foods to their habitual diet and foods that are known to cause GI distress in the individual subjects, and abstaining from supplements, beverages, or food products with live probiotics (e.g., yogurt, kombucha) throughout the study intervention period. Subjects will also be instructed to abstain from exercise and alcohol (24 h) for the following 24 h prior to Visit 3 (Day 21).

At Visit 3 (Day 21), subjects will be instructed to maintain physical activity and habitual diet as much as possible while limiting the introduction of new foods to their habitual diet and foods that are known to cause GI distress in the individual subjects, and abstaining from supplements, beverages, or food products with live probiotics (e.g., yogurt, kombucha) throughout the study intervention period. Subjects will also be instructed to abstain from exercise and alcohol (24 h) for the following 24 h prior to Visit 4 (Day 42).

A query regarding compliance with these instructions will be performed at each visit.

### 3-d Diet Record

Subjects will be dispensed a blank 3-d Diet Record at Visits 1 and 3 (Days −7 and 21) to record all foods and beverages consumed during 3 consecutive days immediately prior to Visits 2 and 4 (Days 0 and 42), respectively. The diet records will be analyzed using the Food Processor® Nutrition Analysis & Fitness Software (version 10.4, or later, Salem, OR) to assess the subject's habitual diet. At Visit 3, subjects will also be dispensed a photocopy of the diet record completed 24 h prior to Visit 2 in order to replicate food/beverage intake for 24 h prior to Visit 4. These two 24 h diet records (i.e., the day prior to Visits 2 and 4) will be compared to ensure consistency in diet at baseline and end of study.

The Bowel Habits Diary (Appendix 4) will provide information on stool frequency and consistency, straining and discomfort during bowel movements, and any sensation of incomplete evacuation. The Bowel Habits Diary will be dispensed at Visits 1 and 3 (Days −7 and 21) to be completed for 3 consecutive days concurrent with the 3-d Diet Record and stool specimen collection prior to Visits 2 and 4 (Days 0 and 42), respectively.

The Gastrointestinal Tolerance Questionnaire (GITQ) contains a series of questions regarding the presence and severity of GI symptoms occurring over the past 7 days. Individual components include the severity of GI symptoms, including gas/flatulence, nausea, vomiting, abdominal cramping, abdominal distention/bloating, borborygmus/stomach rumbling, burping, and/or reflux (heartburn), and these are ranked on a 4-point scale ranging from none-to-severe. Subjects will complete the GITQ at Visit 2 (Day 0) and at Visit 4 (Day 42) prior to the beginning of the GI permeability test.

Subjects will be instructed to collect three fecal samples from one stool specimen produced from one bowel movement during the 3-day period immediately prior to Visits 2 and 4 (Days 0 and 42) according to the instructions provided.

The time and date the samples are collected will be recorded in the eCRF.

### Lactulose/Mannitol GI Permeability Test

At Visit 2 (Day 0) and Visit 4 (Day 42), following an overnight fast (≥10 h) and avoidance of vigorous exercise, NSAIDs, and alcohol for 48 hours, subjects will be instructed to empty their bladder completely. Then, subjects will ingest two sugar probes (1 g 13 C mannitol, and 5 g lactulose) in −240 mL water. Subjects will collect all urine in-clinic for 8 h, with urine collected from 0 to 2 h in one container and from 2:01 h to 8 h in a second container, with the time following complete consumption of the sugar probe as t=0 h. The urine collection containers can remain at room temperature throughout the 8 h collection period, and subjects will be allowed to consume water ad libitum. Female subjects who are menstruating at the time of urine sample collection will be asked to wear a tampon when collecting to minimize sample contamination with blood. A standard lunch will be provided at t=4 h. Subjects will consume the lunch within 30 min and will be instructed to eat until comfortably full. Lunch and water consumption will be recorded, and subjects will be required to replicate at Visit 4 (Day 42).

### Procedures at Each Clinic Visit

The procedures listed will not necessarily be performed in the order described below.

### Screening (Visit 1, Day −7)

Obtain informed consent/HIPAA. Collect medical history. Perform clinic visit procedures: Review prior and current medication/supplement use, Assess inclusion and exclusion criteria, Measure height, Measure body weight, Measure vital signs, BMI calculation, Last menses query, if applicable, Administer in-clinic urine pregnancy test, where applicable, Blood draw for complete chemistry panel, Blood draw for complete hematology profile, Blood draw for hsCRP, Dispense 3-d Diet Record, Dispense stool collection kit, Dispense Bowel Habits Diary, Provide study instructions: Maintain physical activity, Maintain habitual diet as much as possible, with the exception of: Minimize introducing new foods to their habitual diet, as well as foods that are known to cause GI distress in the individual subjects throughout the study, Abstain from pre- and probiotic supplements as well as foods or beverages containing live probiotics (e.g., yogurt, kombucha), fiber supplements). Abstain from vigorous exercise, NSAIDs, and alcohol (48 h) and fast (≥10 h), water only) prior to Visit 2

### Test Visit (Visit 2; Day 0)

Perform clinic visit procedures: Review prior and current medication/supplement use, Review inclusion and exclusion criteria for protocol deviations, Measure body weight, Measure vital signs, Last menses query, if applicable. Query compliance to study instructions, Randomization, Collect and review 3-d Diet Record, Collect Bowel Habits Diary, Collect three fecal samples for possible future analysis of GI microbiome and biomarkers of GI permeability, Blood draw for blood plasma samples for possible future analysis of biomarkers of GI permeability and inflammation, Administer 7-day recall GITQ, Administer two-sugar probe GI permeability test and collect urine in-clinic 8 h, Administer standard lunch at t=4 h, Administer standard snack at t=2 h and 8 h, Dispense study product and have subject consume that day's study product in-clinic, Administer electronic study product log and record that day's study product consumption, Assess AEs (beginning and end of visit). Provide study instructions: Maintain physical activity. Maintain a habitual diet as much as possible, with the exception of: Study product consumption. Minimize introducing new foods to their habitual diet, as well as foods that are known to cause GI distress in the individual subjects throughout the study. Abstain from pre- and probiotic supplements as well as foods or beverages containing live probiotics (e.g., yogurt, kombucha), fiber supplements). Avoid consuming any foods or beverages containing sugar probes used in the intestinal permeability test for 24 h. Abstain from vigorous exercise, NSAIDs, and alcohol (48 h) prior to Visit 3 (Day 21).

### Test Visit (Visit 3; Day 21)

Perform clinic visit procedures: Review prior and current medication/supplement use, Review inclusion and exclusion criteria for protocol deviations, Measure body weight, Measure vital signs, Last menses query, if applicable, Query compliance to study instructions, Blood draw for complete chemistry panel, Blood draw for complete hematology profile, Blood draw for hsCRP, Dispense blank 3-d Diet Record and photocopy of the 24 h diet record from the day prior to Visit 2 for replication during the 24 h prior to Visit 4, Dispense stool collection kit, Dispense Bowel Habits Diary, Collect unused study product and assess compliance, Review electronic study product log, Dispense new study product and have subject consume that day's study product in-clinic, Administer electronic study product log to record that day's study product consumption, Assess AEs, Provide study instructions: Maintain physical activity, Maintain habitual diet as much as possible, with the exception of: Study product consumption, Minimize introducing new foods to their habitual diet, as well as foods that are known to cause GI distress in the individual subjects throughout the study, Abstain from pre- and probiotic supplements as well as foods or beverages containing live probiotics (e.g., yogurt, kombucha), fiber supplements), Avoid consuming any foods or beverages containing sugar probes used in the intestinal permeability test for 24 h, Abstain from vigorous exercise, NSAIDs, and alcohol (48 h) prior to Visit 4 (Day 42).

### Test Visit (Visit 4; Day 42)

Perform clinic visit procedures: Review prior and current medication/supplement use, Review inclusion and exclusion criteria for protocol deviations, Measure body weight, Measure vital signs, Last menses query, if applicable, Query compliance to study instructions, Blood draw for complete chemistry panel, Blood draw for complete hematology profile, Blood draw for hsCRP, Blood draw for plasma samples for possible future analysis of biomarkers of GI permeability and inflammation, Collect and review 3-d Diet Record, Collect Bowel Habits Diary, Collect three fecal samples for possible future analysis of GI microbiome and biomarkers of GI permeability, Collect unused study product and assess compliance, Administer 7-day recall GITQ, Administer two-sugar probe GI permeability test and collect urine in-clinic 8 h, Administer standard lunch at t=4 h, Administer standard snack at t=2 h and 8 h, Assess AEs (beginning and end of visit).

### Data Analysis and Statistical Methods

The difference between placebo and active treatment in the 0-2 h urine ¹³C Mannitol excretion from baseline to Day 42.

### Secondary Outcome Variables

Between placebo and active treatments, the difference in the 2-8 h urine 13 C Mannitol from baseline to Day 42 will be measured. The difference between placebo and active treatment in the 0-2 h urine LMR from baseline to Day 42 will be measured. Between placebo and active treatments, a difference in the 2-8 h urine LMR from baseline to Day 42 will be measured. Between placebo and active treatments, the difference in biomarkers of GI permeability from baseline to Day 42: Blood biomarkers: I-FABP, LBP, Soluble CD14, Fecal biomarkers, Calprotectin, and Secretory IgA will be measured. Between placebo and active treatments, the difference in blood biomarkers of inflammation: CRP, IL-6, IL-1$, and TNF-α will be measured. Between placebo and active treatments, the difference in the 7-day recall GI symptoms from baseline to Day 42 will be measured. The composite score of GITQ (sum of all 8 individual scores), Individual symptom GITQ score, the difference between placebo and active treatments, and difference in bowel function, Stool frequency, and Stool consistency (Bristol stool scale) will be measured. Between placebo and active treatments, differences in fecal microbiota taxonomic profiles from baseline to Day 42 (e.g., within-intervention alpha-diversity, within-subject alpha-diversity, within-intervention beta-diversity, Bray-Curtis dissimilarity, relative change in taxa abundance) and metagenomics-informed metabolic or functional profiles will be measured. Safety profiles, chemistry panel, hematology profile, vital signs, body weight, and incidence of AEs will be assessed.

### Sample Size

Previous research on dietary interventions to modulate gut permeability has focused on changes in the LMR; however, it is believed that the changes in lactulose will be negligible and thus driven by the 13 C mannitol excretion. Therefore, the primary comparison of interest is the 13 C Mannitol from 0-2 h. While there is limited data on what is a clinically meaningful difference in 13 C mannitol excretion, the response is expected to be similar to the LMR. Assuming the common standard deviation (SD) of LMR to be approximately 0.008, about a 35.0% reduction from the control LMR ratio of 0.020 would yield an effect size of about 0.86. With a desired statistical power of 90% and using a 1:1 allocation with a 3-arm parallel study, a sample size of 105 subjects (n=35 per arm) is sufficient to detect the estimated effect size using a 2-sided t-test with adjustment for multiple comparisons α=0.025 (each active arm compared to placebo). The sample size will be increased to N=126 (n=42 per arm) to account for ˜20% attrition.

### Statistical Analysis

Statistical analysis details for this study will be provided in a Statistical Analysis Plan. Briefly, all statistical analyses will be conducted using SAS for Windows (version 9.4, or higher, Cary, NC) and/or R 3.3.1 (R Core Team 2016). Analysis populations will include intent-to-treat (ITT; all subjects randomized in the study who provide at least one outcome data point) and per protocol (PP; all subjects completing the study in compliance). The ITT population will serve as the primary analysis population. Subjects may be excluded from the PP population for non-compliance, which includes but is not limited to Missing appointments, Use of prohibited drugs, or any products thought to alter the primary outcome variable during the study <80% or >120% compliance with study product consumption, and Not adhering to instructions as outlined in the protocol. All decisions regarding subject population and data inclusion will be documented prior to database lock.

### Baseline Characteristics

Descriptive statistics [number of subjects, mean, standard deviation (SD), median, interquartile limits, minimum and maximum or frequency counts] will be presented for subject demographics and anthropometric measurements collected at screening/baseline for all analysis populations.

### Outcome Analysis

Descriptive statistics (i.e., number of subjects, minimum and maximum, median, interquartile limits, mean, and standard deviation) will be presented for all the continuous outcomes for each test group. All tests of significance, unless otherwise stated, will be performed at alpha=0.05, two-sided.

Continuous outcomes measured at baseline and day 42 will be analyzed following an analysis of covariance (ANCOVA) approach. Note that the estimate of the treatment effect is computationally identical to an analysis that utilizes the change score as the response variable adjusted for baseline, intervention group, and stratification factor. However, if necessary, the Day 42 response variable will be selected to allow for easier variance-stabilizing transformations. The model will contain terms for baseline 0-2 hr urine LMR, intervention group, and the stratification factor hsCRP group. For each model, the residuals will be analyzed visually (QQ-plots, residual plots) to verify model assumptions (normality, constant variance, homogeneity) are met. If necessary, a transformation (i.e. log) may be considered. The model-derived pairwise comparison between each active group and placebo will be estimated using least squares means and tested for significance with a t-test. The model-derived Day 42 mean estimate and the corresponding 97.5% confidence interval will be estimated for each group.

The response profile for the continuous outcomes (stool frequency, BSS, and hsCRP) measured at baseline, day 21, and day 42 will be analyzed with a repeated measures model. The model will contain fixed effects for time point, intervention group, time point by intervention group interaction, and the stratification factor hsCRP group. The change from baseline to each follow-up time point will be evaluated for each safety measure, including the Chemistry Panel and Hematology Panel. Within and between-group differences will be evaluated with the Wilcoxon signed rank and rank sum tests, respectively. Additional analysis details will be specific in the statistical analysis plan (SAP) developed for this protocol. Clinical Monitoring

An initiation meeting will be conducted. The protocol, eCRFs, and pertinent aspects of GCP will be reviewed with the Clinical Investigator and all study staff at this meeting. Remote and on-site monitoring visits will be conducted during the study, focusing on the trial's human participant protection and data integrity risks. A clinical monitoring plan will be developed, which identifies specific risk-based monitoring focal points. These may include informed consent, eligibility criteria, SAEs, serious protocol violations, endpoints, test article administration, and accountability.

This invention is, however, susceptible to modifications and alternate constructions from that discussed above that are fully equivalent. Consequently, this invention is not limited to the particular embodiments disclosed. On the contrary, this invention covers all modifications and alternate constructions coming within the spirit and scope of the invention as generally expressed by the following claims, which particularly point out and distinctly claim the subject matter of the invention. While the disclosure has been illustrated and described in detail in the drawings and foregoing description, such illustration and description are to be considered illustrative or exemplary and not restrictive.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article ‘a’ or ‘an’ does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases ‘at least one’ and “one or more’ to introduce claim recitations. However, the use of such phrases should not be construed to imply that the introduction of a claim recitation by the indefinite articles ‘a’ or ‘an’ limits any particular claim containing such introduced claim recitation to embodiments containing only one such recitation, even when the same claim includes the introductory phrases ‘one or more’ or ‘at least one’ and indefinite articles such as ‘a’ or ‘an’ (e.g., ‘a’ and/or ‘an’ should typically be interpreted to mean ‘at least one’ or ‘one or more’); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of ‘two recitations,’ without other modifiers, typically means at least two recitations, or two or more recitations). Furthermore, in those instances where a convention analogous to ‘at least one of A, B, and C, etc.’ is used, in general such a construction is intended in the sense one having skill in the art would understand the convention (e.g., ‘a system having at least one of A, B, and C’ would include but not be limited to systems that have A alone, B alone, C alone, A and B together, A and C together, B and C together, and/or A, B, and C together, etc.). In those instances where a convention analogous to ‘at least one of A, B, or C, etc.’ is used, in general such a construction is intended in the sense one having skill in the art would understand the convention (e.g., ‘a system having at least one of A, B, or C’ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase ‘A or B’ will be understood to include the possibilities of ‘A’ or ‘B’ or ‘A and B.’

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed is:

1. A method for treating, improving or ameliorating a disease or condition associated with intestinal permeability, improving intestinal permeability, or improving gut barrier function in a subject, the method comprising:
providing to the subject a composition comprising at least one carrier and an effective amount of a compound of Formula (I), 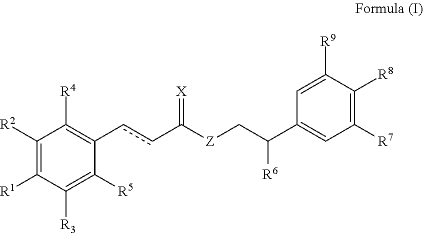
wherein
R¹, R², R³, R⁴, R⁵, R⁶, R⁷, R⁸, and R⁹ are each independently selected from hydrogen, deuterium, hydroxyl, halogen, cyano, nitro, optionally substituted amino, optionally substituted C-amido, optionally substituted N-amido, optionally substituted ester, optionally substituted —C₁₋₆alkyl, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl;
the dashed bond is present or absent;
X is CH₂ or O;
Z is CHR^{a}, NR^{a}, or O; and
R^{a} is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl,
thereby treating, improving or ameliorating a disease or condition associated with intestinal permeability in the subject or improving a digestive function.

2. The method of claim 1, wherein the composition is formulated as a dietary supplement, food ingredient or additive, a food product, a medical food, nutraceutical or pharmaceutical composition.

3. The method of claim 1 or 2, wherein the composition of Formula (I) is in a unit dosage form and is configured for administration between 0.1 and 100 mg/kg of the body weight of the subject of Formula (I) per administration.

4. The method of any one of claims 1 to 3, wherein the compound of Formula (I) is selected from the group consisting of: N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, N-trans-chlorogenoyltyramine, N-trans-rosmarinoyltyramine, and 5-hydroxyferuloyltyramine, or a pharmaceutically acceptable salt, solvates, and combinations of the foregoing.

5. The method of claim 4, wherein the compound of Formula (I) is N-trans-caffeoyltyramine and further comprises N-trans-feruloyltyramine.

6. The method of claim 5, wherein the N-trans-caffeoyltyramine and the N-trans-feruloyltyramine is in a ratio from about 10:1 to about 1:10.

7. The method of claim 5 or 6, wherein the N-trans-caffeoyltyramine and the N-trans-feruloyltyramine is in a ratio from about 5:1 to about 1:5.

8. The method of any one of claims 5 to 7, wherein the N-trans-caffeoyltyramine and the N-trans-feruloyltyramine is in a ratio from about 2.5:1 to about 2:1.

9. The method of any one of claims 5 to 8, wherein the N-trans-caffeoyltyramine and the N-trans-feruloyltyramine is in a ratio from about 2.2:1.

10. The method of any one of claims 1 to 9, wherein the disease or condition associated with intestinal permeability in the subject is associated with a gastrointestinal epithelial cell barrier function disorder.

11. The method of claim 10, wherein the gastrointestinal epithelial cell barrier function disorder is a disease associated with decreased intestinal epithelium integrity.

12. The method of claim 10, wherein the gastrointestinal epithelial cell barrier function disorder is at least one selected from the group consisting of: inflammatory bowel disease, Crohn's disease, ulcerative colitis, pouchitis, irritable bowel syndrome, enteric infections, Clostridium difficile infections, metabolic diseases, obesity, type 2 diabetes, non-alcoholic steatohepatitis, non-alcoholic fatty liver disease, liver disorders, alcoholic steatohepatitis, celiac disease, necrotizing enterocolitis, gastro intestinal disorders, short bowel syndrome, GI mucositis, chemotherapy induced mucositis, radiation induced mucositis, oral mucositis, interstitial cystitis, neurological disorders, cognitive disorders, Alzheimer's, Parkinson's, multiple sclerosis, autism, chemotherapy associated steatohepatitis (CASH), and pediatric versions of the aforementioned diseases.

13. The method of claim 12, wherein the gastrointestinal epithelial cell barrier function disorder is inflammatory bowel disease.

14. The method of claim 12, wherein the gastrointestinal epithelial cell barrier function disorder is Crohn's disease.

15. The method of claim 12, wherein the gastrointestinal epithelial cell barrier function disorder is ulcerative colitis.

16. The method of any one of claims 1 to 15, wherein administering comprises rectal, parenteral, intravenous, topical, oral, dermal, transdermal, or subcutaneous administration.

17. The method of any one of claims 1 to 16, wherein the subject experiences a reduction in at least one symptom associated with the gastrointestinal epithelial cell barrier function disorder selected from the group consisting of: abdominal pain, blood in stool, pus in stool, fever, weight loss, frequent diarrhea, fatigue, reduced appetite, tenesmus, and rectal bleeding.

18. The method of any one of claims 1 to 17, further comprising: administering at least one second therapeutic agent to the subject, said second therapeutic agent selected from the group consisting of: an anti-diarrheal, a 5-aminosalicylic acid compound, an antiinflammatory agent, an antibiotic, an antibody, an anti-cytokine agent, an antiinflammatory cytokine agent, a steroid, a corticosteroid, and an immunosuppressant.

19. The method of any one of claims 1 to 18, wherein treating or improving a disease or condition associated with intestinal permeability in a subject improves digestive health in a subject by at least 10%.

20. The method of any one of claims 1 to 19, wherein treating or improving a disease or condition associated with intestinal permeability in a subject reduces a disease or condition in a subject by at least 10%.

21. The method of any one of claims 1 to 20, wherein the compound of Formula I is an extract of a plant.

22. The method of claim 21, wherein the extract of the plant is selected from the group comprising Allium, Amoracia, Chenopodium, Cannabis, Spinacia, Fagopyrum, Annona, Jatropha, Piper, Eragrostis, Zea, Nelumbo, Ipomoea, Capsicum, Lycium, Solanum, and Tribulus.

23. The method of claim 21, wherein the extract of the plant is hemp hulls, peppercorn, or black seed.

24. A consumable composition for treating a disease or condition associated with intestinal permeability or improving impaired gut barrier function comprising at least one carrier and an effective amount of a compound of Formula (I) or a pharmaceutically acceptable salt: 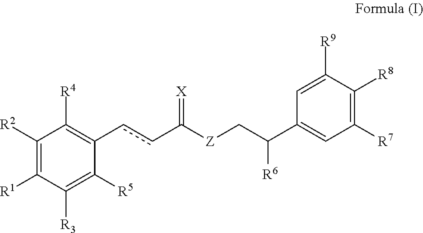
wherein
R¹, R², R³, R⁴, R⁵, R⁶, R⁷, R⁸, and R⁹ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆ alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl;
the dashed bond is present or absent;
X is CH₂ or O;
Z is CHR^{a}, NR^{a}, or O; and
R^{a} is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

25. The consumable composition of claim 24, wherein the composition is formulated as a dietary supplement, food ingredient or additive, a food product, a medical food, nutraceutical or pharmaceutical composition.

26. The consumable composition of claim 24 or 25, wherein the composition of Formula (I) is in a unit dosage form and is configured for administration between 0.1 and 100 mg/kg of the body weight of the subject of Formula (I) per administration.

27. The consumable composition of any one of claims 24 to 26, wherein the compound of Formula (I) is selected from the group consisting of: N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, N-trans-chlorogenoyltyramine, N-trans-rosmarinoyltyramine, and 5-hydroxyferuloyltyramine, or a pharmaceutically acceptable salt, solvates, and combinations of the foregoing.

28. The consumable composition of claim 27, wherein the compound of Formula (I) is N-trans-caffeoyltyramine and further comprises N-trans-feruloyltyramine.

29. The consumable composition of claim 28, wherein the N-trans-caffeoyltyramine and the N-trans-feruloyltyramine is in a ratio from about 10:1 to about 1:10.

30. The consumable composition of claim 28 or 29, wherein the N-trans-caffeoyltyramine and the N-trans-feruloyltyramine is in a ratio from about 5:1 to about 1:5.

31. The consumable composition of any one of claims 28 to 30, wherein the N-trans-caffeoyltyramine and the N-trans-feruloyltyramine is in a ratio from about 2.5:1 to about 2:1.

32. The consumable composition of any one of claims 28 to 31, wherein the N-trans-caffeoyltyramine and the N-trans-feruloyltyramine is in a ratio from about 2.2:1.

33. The consumable composition of any one of claims 25 to 32, wherein the food product is a food bar.

34. The consumable composition of claim 33, wherein the food bar comprises from about 0.01% to about 20% (w/w) N-trans-caffeoyltyramine.

35. The consumable composition of claim 33 or 34, wherein the food bar comprises from about 0.01% to about 20% (w/w) N-trans-feruloyltyramine.

36. The consumable composition of claim 33 to 35, wherein the food product comprises at least 10% fiber.

37. The consumable composition of any one of claims 25 to 32, wherein the food product is a crisp.

38. The consumable composition of claim 37, wherein the food product comprises from about 0.01% to about 20% (w/w) N-trans-caffeoyltyramine.

39. The consumable composition of claim 37 or 38, wherein the food product comprises from about 0.01% to about 20% (w/w) N-trans-feruloyltyramine.

40. The consumable composition of claim 37 to 39, wherein the food product comprises at least 10% fiber.

41. A method for improving transepithelial electrical resistance in a cell, the method comprising:
contacting a cell with a composition comprising at least one carrier and an effective amount of a compound of Formula (I), 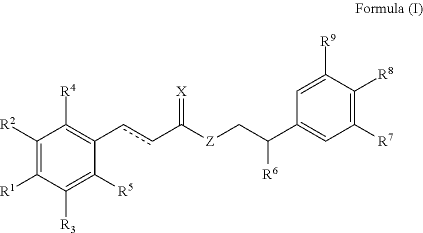
wherein
R¹, R², R³, R⁴, R⁵, R⁶, R⁷, R⁸, and R⁹ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl;
the dashed bond is present or absent;
X is CH₂ or O;
Z is CHR^{a}, NR^{a}, or O; and
R^{a} is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆aylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

42. The method of claim 41, wherein the compound of Formula (I) is selected from the group consisting of: N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, N-trans-chlorogenoyltyramine, N-trans-rosmarinoyltyramine, and 5-hydroxyferuloyltyramine, or a pharmaceutically acceptable salt, solvates, and combinations of the foregoing.

43. The method of claim 41 or 42, wherein the cell is in vivo.

44. The method of claim 41 or 42, wherein the cell is in vitro.

45. The method of claim 41 to 44, wherein the cell is mammalian.

46. The method of claim 42, wherein the compound of Formula (I) is N-trans-caffeoyltyramine and further comprises N-trans-feruloyltyramine.

47. The method of claim 46, wherein the N-trans-caffeoyltyramine and the N-trans-feruloyltyramine is in a ratio from about 10:1 to about 1:10.

48. The method of claim 46 or 47, wherein the N-trans-caffeoyltyramine and the N-trans-feruloyltyramine is in a ratio from about 5:1 to about 1:5.

49. The method of any one of claims 46 to 48, wherein the N-trans-caffeoyltyramine and the N-trans-feruloyltyramine is in a ratio from about 2.5:1 to about 2:1.

50. The method of any one of claims 46 to 49, wherein the N-trans-caffeoyltyramine and the N-trans-feruloyltyramine is in a ratio from about 2.2:1.
